(19) United States
(12) Patent Application Publication
(10) Pub. No.: US 2006/0270667 A1
(43) Pub. Date: Nov. 30, 2006

(54) NOVEL MEDICAMENT COMBINATIONS FOR THE TREATMENT OF RESPIRATORY DISEASES

(75) Inventors: Michael P. PIEPER, Biberach (DE); Andreas Schnapp, Biberach (DE); Peter Nickolaus, Warthausen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/420,872

(22) Filed: May 30, 2006

(30) Foreign Application Priority Data
May 31, 2005 (EP) 05104702

(51) Int. Cl. A61K 31/538; A61K 31/4745; A61K 31/46; A61K 31/4184; A61K 31/138

(52) U.S. Cl. 514/230.5; 514/521; 514/651; 514/394; 514/312; 514/367; 514/305; 514/649; 514/643; 514/291; 514/304

(57) ABSTRACT

The present invention relates to new medicament combinations which contain in addition to one or more, preferably one, betamimetic 1, at least one anticholinergic 2 and at least one PDEIV-inhibitor 3, processes for preparing them and their use as pharmaceutical compositions.

The present invention relates to new medicament combinations which contain in addition to one or more, preferably one, betamimetic 1, at least one anticholinergic 2 and at least one PDEIV-inhibitor 3, processes for preparing them and their use as pharmaceutical compositions.

### BRIEF DECSRIPTION OF THE DRAWING

FIG. 1 shows a particularly preferred inhaler for using capsules containing the pharmaceutical combination according to the invention.

### DETAILED DESCRIPTION OF THE INVENTION

The present invention relates to medicament combinations which contain in addition to one or more, preferably one, betamimetic 1, at least one anticholinergic 2 and at least one PDEIV-inhibitor 3, optionally together with a pharmaceutically acceptable excipient.

Within the instant application the term betamimetic is optionally also replaced by the term beta₂-agonist, and these terms are considered equivalent in the context of the present invention. According to the instant invention preferred beta₂ agonists 1 in the combinations according to the invention are selected from the group consisting of albuterol (1.1), bambuterol (1.2), bitolterol (1.3), broxaterol (1.4), carbuterol (1.5), clenbuterol (1.6), fenoterol (1.6), formoterol (1.8), hexoprenaline (1.9), ibuterol (1.10), isoetharine (1.11), isoprenaline (1.12), levosalbutamol (1.13), mabuterol (1.14), meluadrine (1.15), metaproterenol (1.16), orciprenaline (1.17), pirbuterol (1.18), procaterol (1.19), reproterol (1.20), TD 3327 (1.21), ritodrine (1.22), salmeterol (1.23), salmefamol (1.24), soterenot (1.25), sulphonterol (1.26), tiaramide (1.27), terbutaline (1.28), tolubuterol (1.29), CHF-4226 (=TA 2005 or carmoterol; 1.30), HOKU-81 (1.31), KUL-1248 (1.32), 3-(4-{6-[2-Hydroxy-2-(4-hydroxy-3-hydroxymethyl-phenyl)-ethylamino]-hexyloxy}-butyl)-benzenesulfoneamide (1.33), 5-[2-(5,6-Diethyl-indan-2-ylamino)-1-hydroxy-ethyl]-8-hydroxy-1H-quinolin-2-one (1.34), 4-hydroxy-7-[2-{[2-{[3-(2-phenylethoxy)propyl]sulphonyl}ethyl]-amino}ethyl]-2(3H)-benzothiazolone (1.35), 1-(2-fluoro-4-hydroxyphenyl)-2-[4-(1-benzimidazolyl)-2-methyl-2-butylamino]ethanol (1.36), 1-[3-(4-methoxybenzyl-amino)-4-hydroxyphenyl]-2-[4-(1-benzimidazolyl)-2-methyl-2-butylamino]ethanol (1.37), 1-[2H-5-hydroxy-3-oxo-4H-1,4-benzoxazin-8-yl]-2-[3-(4-N,N-dimethylaminophenyl)-2-methyl-2-propylamino]ethanol (1.38), 1-[2H-5-hydroxy-3-oxo-4H-1,4-benzoxazin-8-yl]-2-[3-(4-methoxyphenyl)-2-methyl-2-propylamino]ethanol (1.39), 1-[2H-5-hydroxy-3-oxo-4H-1,4-benzoxazin-8-yl]-2-[3-(4-n-butyloxyphenyl)-2-methyl-2-propylamino]ethanol (1.40), 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol (1.41), 5-hydroxy-8-(1-hydroxy-2-isopropylaminobutyl)-2H-1,4-benzoxazin-3-(4H)-one (1.42), 1-(4-amino-3-chloro-5-trifluormethylphenyl)-2-tert.-butylamino)ethanol (1.43), 1-(4-ethoxycarbonylamino-3-cyano-5-fluorophenyl)-2-(tert.-butylamino)ethanol (1.44), and N-[2-Hydroxy-5-(1-hydroxy-2-{2-[4-(2-hydroxy-2-phenyl-ethylamino)-phenyl]-ethylamino}-ethyl)-phenyl]-formamide (14.5), optionally in the form of the racemates, the enantiomers, the diastereomers and optionally the pharmacologically acceptable acid addition salts and the hydrates thereof.

According to the instant invention more preferred beta₂ agonists 1 in the combinations according to the invention are selected from the group consisting of bambuterol (1.2), bitolterol (1.3), carbuterol (1.5), clenbuterol (1.6 fenoterol (1.7) formoterol (1.8), hexoprenaline (1.9), ibuterol (1.10), pirbuterol (1.8), procaterol (1.19), reproterol (1.20), TD 3327 (1.21), salmeterol (1.23), sulphonterol (1.26), terbutaline (1.28), tolubuterol (1.29), CHF-4226 (=TA 2005 or carmoterol; 1.30), 3-(4-{6-[2-Hydroxy-2-(4-hydroxy-3-hydroxymethyl-phenyl)-ethylamino]-hexyloxy}-butyl)-benzenesulfoneamide (1.33), 5-[2-(5,6-Diethyl-indan-2-ylamino)-1-hydroxy-ethyl]-8-hydroxy-1H-quinolin-2-one (1.34), 4-hydroxy-7-[2-{[2-{[3-(2-phenylethoxy)propyl]sulphonyl}ethyl]-amino}ethyl]-2(3H)-benzothiazolone (1.35), 1-(2-fluoro-4-hydroxyphenyl)-2-[4-(1-benzimidazolyl)-2-methyl-2-butylamino]ethanol (1.36), 1-[3-(4-methoxybenzyl-amino)-4-hydroxyphenyl]-2-[4-(1-benzimidazolyl)-2-methyl-2-butylamino]ethanol (1.37), 1-[2H-5-hydroxy-3-oxo-4H-1,4-benzoxazin-8-yl]-2-[3-(4-N,N-dimethylaminophenyl)-2-methyl-2-propylamino]ethanol (1.38), 1-[2H-5-hydroxy-3-oxo-4H-1,4-benzoxazin-8-yl]-2-[3-(4-methoxyphenyl)-2-methyl-2-propylamino]ethanol (1.39), 1-[2H-5-hydroxy-3-oxo-4H-1,4-benzoxazin-8-yl]-2-[3-(4-n-butyloxyphenyl)-2-methyl-2-propylamino]ethanol (1.40), 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol (1.41), 5-hydroxy-8-(1-hydroxy-2-isopropylaminobutyl)-2H-1,4-benzoxazin-3-(4H)-one (1.42), 1-(4-amino-3-chloro-5-trifluormethylphenyl)-2-tert.-butylamino)ethanol (1.43), 1-(4-ethoxycarbonylamino-3-cyano-5-fluorophenyl)-2-(tert.-butylamino)ethanol (1.44), and N-[2-Hydroxy-5-(1-hydroxy-2-{2-[4-(2-hydroxy-2-phenyl-ethylamino)-phenyl]-ethylamino}-ethyl)-phenyl]-formamide (1.45), optionally in the form of the racemates, the enantiomers, the diastereomers and optionally the pharmacologically acceptable acid addition salts and the hydrates thereof.

More preferably, the betamimetics 1 used as within the compositions according to the invention are selected from the group consisting of fenoterol (1.7), formoterol (1.8), salmeterol (1.23), CHF-4226 (=TA 2005 or carmoterol; 1.30), 3-(4-{6-[2-Hydroxy-2-(4-hydroxy-3-hydroxymethyl-phenyl)-ethylamino]-hexyloxy}-butyl)-benzenesulfoneamide (1.33), 5-[2-(5,6-Diethyl-indan-2-ylamino)-1-hydroxy-ethyl]-8-hydroxy-1H-quinolin-2-one (1.34), 1-[3-(4-methoxybenzyl-amino)-4-hydroxyphenyl]-2-[4-(1-benzimidazolyl)-2-methyl-2-butylamino]ethanol (1.37), 1-[2H-5-hydroxy-3-oxo-4H-1,4-benzoxazin-8-yl]-2-[3-(4-N,N-dimethylaminophenyl)-2-methyl-2-propylamino]ethanol (1.38), 1-[2H-5-hydroxy-3-oxo-4H-1,4-benzoxazin-8-yl]-2-[3-(4-methoxyphenyl)-2-methyl-2-propylamino]ethanol (1.39), 1-[2H-5-hydroxy-3-oxo-4H-1,4-benzoxazin-8-yl]-2-[3-(4-n-butyloxyphenyl)-2-methyl-2-propylamino]ethanol (1.40), 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol (1.41), and N-[2-Hydroxy-5-(1-hydroxy-2-{2-[4-(2-hydroxy-2-phenyl-ethylamino)-phenyl]-ethylamino}-ethyl)-phenyl]-formamide (1.45), optionally in the form of the racemates, the enantiomers, the diastereomers and optionally the pharmacologically acceptable acid addition salts and the hydrates thereof. Of the betamimetics mentioned above the compounds formoterol (1.8), salmeterol (1.23), CHF-4226 (=TA 2005 or carmoterol; 1.30), 3-(4-{6-[2-Hydroxy-2-(4-hydroxy-3-hydroxymethyl-phenyl)-ethylamino]-hexyloxy}-butyl)-benzenesulfoneamide (1.33), 5-[2-(5,6-Diethyl-indan-2-ylamino)-1-hydroxy-ethyl]-8-hydroxy-1H-quinolin-2-one are (1.34), and N-[2-Hydroxy-5-(1-hydroxy-2-{2-[4-(2-hydroxy-2-phenyl-ethylamino)-phenyl]-ethylamino}-ethyl)-phenyl]-formamide (1.45), particularly preferred, optionally in the form of the racemates, the enantiomers, the diastereomers and optionally the pharmacologically acceptable acid addition salts thereof, and the hydrates thereof.

Examples of pharmacologically acceptable acid addition salts of the betamimetics 1 according to the invention are the pharmaceutically acceptable salts which are selected from among the salts of hydrochloric acid, hydrobromic acid, sulphuric acid, phosphoric acid, methanesulphonic acid, acetic acid, fumaric acid, succinic acid, lactic acid, citric acid, tartaric acid, 1-hydroxy-2-naphthalenecarboxylic acid, 4-phenylcinnamic acid, 5-(2,4-difluorophenyl)salicylic acid or maleic acid. If desired, mixtures of the abovementioned acids may also be used to prepare the salts 1.

According to the invention, the salts of the betamimetics 1 selected from among the hydrochloride, hydrobromide, sulphate, phosphate, fumarate, methanesulphonate, 4-phenylcinnamate, 5-(2,4-difluorophenyl)salicylate, maleate and xinafoate are preferred. Particularly preferred are the salts of 1 in the case of salmeterol selected from among the hydrochloride, sulphate, 4-phenylcinnamate, 5-(2,4-difluorophenyl)salicylate and xinafoate, of which the 4-phenylcinnamate, 5-(2,4-difluorophenyl)salicylate and especially xinafoate are particularly important. Particularly preferred are the salts of 1 in the case of formoterol selected from the hydrochloride, sulphate, hemifumarate and fumarate, of which the hydrochloride, hemifumarate and fumarate are particularly preferred. Of exceptional importance according to the invention is formoterol fumarate dihydrate or formoterol hemifumarate hydrate.

Any reference to the term betamimetics 1 also includes a reference to the relevant enantiomers or mixtures thereof.

In the pharmaceutical compositions according to the invention, the compounds 1 may be present in the form of their racemates, enantiomers or mixtures thereof. The separation of the enantiomers from the racemates may be carried out using methods known in the art (e.g. by chromatography on chiral phases, etc.) If the compounds 1 are used in the form of their enantiomers, it is particularly preferable to use the enantiomers in the R configuration at the C—OH group. If the compounds 1 possess 2 chiral carbon atoms they are preferably used in the form of their pure diastereomers, particularly in the form of those diasteromers that possess R configuration at the C—OH group. An example may be R,R-formoterol.

In the medicament combinations according to the invention the anticholinergic 2 is preferably selected from among the tiotropium salts (2.1), oxitropium salts (2.2), flutropium salts (2.3), ipratropium salts (2.4), glycopyrronium salts (2.5), trospium salts (2.6) and the compounds of formulae 2.7 to 2.13.

In the above-mentioned salts 2.1 to 2.6 the cations tiotropium, oxitropium, flutropium, ipratropium, glycopyrronium and trospium are the pharmacologically active constituents. Explicit references to the above-mentioned cations are indicated by the numerals 2.1′ to 2.6′. Each reference to the above-mentioned salts 2.1 to 2.6 naturally includes a reference to the corresponding cations tiotropium (2.1′), oxitropium (2.2′), flutropium (2.3′), ipratropium (2.4′), glycopyrronium (2.5′) and trospium (2.6′).

By the salts 2.1 to 2.6 are meant according to the invention those compounds which contain in addition to the cations tiotropium (2.1′), oxitropium (2.2′), flutropium (2.3′), ipratropium (2.4′), glycopyrronium (2.5′) and trospium (2.6′) as counter-ion (anion) chloride, bromide, iodide, sulphate, phosphate, methanesulphonate, nitrate, maleate, acetate, citrate, fumarate, tartrate, oxalate, succinate, benzoate or p-toluenesulphonate contain, while the chloride, bromide, iodide, sulphate, methanesulphonate or p-toluenesulphonate are preferred as counter-ions. Of all the salts the chloride, bromide, iodide and methanesulphonate are particularly preferred.

In the case of the trospium salts (2.6) the chloride is particularly preferred. Of the other salts 2.1 to 2.5 the methanesulphonates and bromides are of particular importance.

Of particular importance are medicament combinations which contain tiotropium salts (2.1), oxitropium salts (2.2) or ipratropium salts (2.4), while the respective bromides are particularly important according to the invention. Of particular importance is the tiotropium bromide (2.1). The above-mentioned salts may optionally be present in the medicament combinations according to the invention in the form of their solvates or hydrates, preferably in the form of their hydrates. In the case of tiotropium bromide the medicament combinations according to the invention preferably contain this in the form of the crystalline tiotropium bromide monohydrate, which is known from WO 02/30928. If the tiotropium bromide is used in anhydrous form in the medicament combinations according to the invention, it is preferable to use the anhydrous crystalline tiotropium bromide which is known from WO 03/000265.

The above-mentioned anticholinergics optionally have chiral carbon centres. In this case the medicament combinations according to the invention may contain the anticholinergics in the form of their enantiomers, mixtures of enantiomers or racemates, while enantiomerically pure anticholinergics as for instance R,R-glycopyrrolate (2.5) are preferably used.

In another preferred embodiment of the present invention the anticholinergics 2 contained in the medicament combinations according to the invention are selected from the salts of formula 2.7 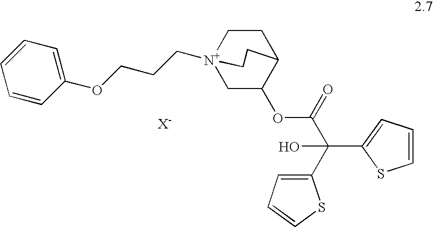
wherein
X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate,
   optionally in the form of the racemates, enantiomers or hydrates thereof.

Preferred medicament combinations contain salts of formula 2.7, wherein
X⁻ denotes an anion with a single negative charge, preferably an anion selected from among the fluoride, chloride, bromide, methanesulphonate and p-toluenesulphonate, preferably bromide,
   optionally in the form of the racemates, enantiomers or hydrates thereof.

Preferred medicament combinations contain salts of formula 2.7, wherein
X⁻ denotes an anion with a single negative charge, preferably an anion selected from among the chloride, bromide and methanesulphonate, preferably bromide,
   optionally in the form of the racemates, enantiomers or hydrates thereof.

Particularly preferred medicament combinations contain the compound of formula 2.7 in the form of the bromide.

Of particular importance are those medicament combinations which contain the enantiomers of formula 2.7-en 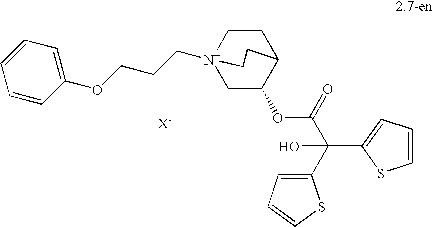
wherein X⁻ may have the above-mentioned meanings.

In another preferred embodiment of the present invention the anticholinergics 2 contained in the medicament combinations according to the invention are selected from the salts of formula 2.8 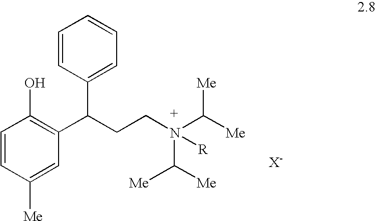
wherein R denotes either methyl (2.8.1) or ethyl (2.8.2 and wherein X⁻ may have the above-mentioned meanings. In an alternative embodiment the compound of formula 2.8 is present in the form of the free base 2.8-base

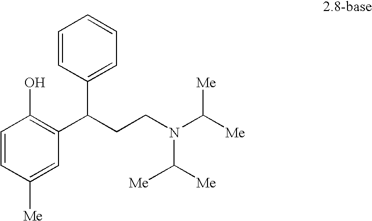

The medicament combinations according to the invention may contain the anticholinergic of formula 2.8 (or 2.8-base) in the form of the enantiomers, mixtures of enantiomers or racemates thereof. Preferably the anticholinergics of formula 2.8 (or 2.8-base) are present in the form of their R-enantiomers.

In another preferred embodiment of the present invention the anticholinergics 2 contained in the medicament combinations according to the invention are selected from the compounds of formula 2.9 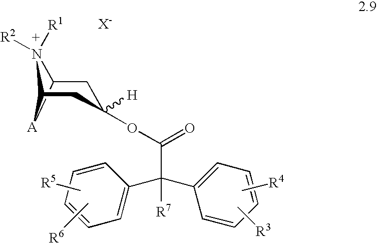
wherein
A denotes a double-bonded group selected from the groups 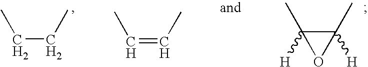
X⁻ denotes one of the above-mentioned anions with a single negative charge, preferably chloride, bromide or methanesulphonate,
R¹ and R² which may be identical or different denote a group selected from methyl, ethyl, n-propyl and iso-propyl, which may optionally be substituted by hydroxy or fluorine, preferably unsubstituted methyl;
R³, R⁴, R⁵ and R⁶, which may be identical or different denote hydrogen, methyl, ethyl, methyloxy, ethyloxy, hydroxy, fluorine, chlorine, bromine, CN, CF₃ or NO₂;
R⁷ denotes hydrogen, methyl, ethyl, methyloxy, ethyloxy, —CH₂—F, —CH₂—CH₂—F, —O—CH₂—F, —O—CH₂—CH₂—F, —CH₂—OH, —CH₂—CH₂—OH, CF₃, —CH₂—OMe, —CH₂—CH₂-OMe, —CH₂—OEt, —CH₂—CH₂-OEt, —O—COMe, —O—COEt, —O—COCF₃, —O—COCF₃, fluorine, chlorine or bromine.

The compounds of formula 2.9 are known in the art (WO 02/32899).

Within the scope of the medicament combinations according to the invention preferred compounds of formula 2.9 are those wherein
X⁻ denotes bromide;
R¹ and R² which may be identical or different, denote methyl or ethyl, preferably methyl;
R³, R⁴, R⁵ and R⁶, which may be identical or different, denote hydrogen, methyl, methyloxy, chlorine or fluorine;
R⁷ denotes hydrogen, methyl or fluorine.

Of particular importance are medicament combinations which contain compounds of formula 2.9, wherein
A denotes a double-bonded group selected from

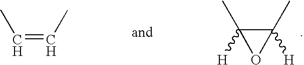

Of particular importance are those medicament combinations which contain in addition to a compound of formula 1 one of the following compounds of formula 2.9:
tropenol 2,2-diphenylpropionate methobromide (2.9.1),
scopine 2,2-diphenylpropionate methobromide (2.9.2),
scopine 2-fluoro-2,2-diphenylacetate methobromide (2.9.3),
tropenol 2-fluoro-2,2-diphenylacetate methobromide (2.9.4);

The compounds of formula 2.9 may optionally in the form of the enantiomers, mixtures of enantiomers or racemates thereof, as well as optionally in the form of the hydrates and/or solvates thereof.

In another preferred embodiment of the present invention the anticholinergics 2 contained in the medicament combinations according to the invention are selected from the compounds of formula 2.10 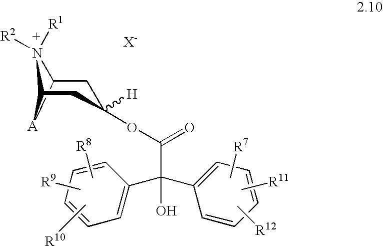
wherein
A, X⁻, R¹ and R² may have the meanings given above and wherein
R⁷, R⁸, R⁹, R¹⁰, R¹¹ and R¹², which may be identical or different, denote hydrogen, methyl, ethyl, methyloxy, ethyloxy, hydroxy, fluorine, chlorine, bromine, CN, CF₃ or NO₂, while at least one of the groups R⁷, R⁸, R⁹, R¹⁰, R¹¹ and R¹² may not be hydrogen.

The compounds of formula 2.10 are known in the art (WO 02/32898).

Within the scope of the medicament combinations according to the invention preferred compounds of formula 2.10 are those wherein
A denotes a double-bonded group selected from 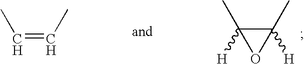
X⁻ bromide;
R¹ and R² which may be identical or different, denote methyl or ethyl, preferably methyl;
R⁷, R⁸, R⁹, R¹⁰, R¹¹ and R¹², which may be identical or different, denote hydrogen, fluorine, chlorine or bromine, preferably fluorine, while at least one of the groups R⁷, R⁸, R⁹, R¹⁰, R¹¹ and R¹² may not be hydrogen.

Of particular importance are those medicament combinations which contain in addition to a compound of formula 1 one of the following compounds of formula 2.10:
tropenol 3,3′,4,4′-tetrafluorobenzilate methobromide (2.10.1),
scopine 3,3′,4,4′-tetrafluorobenzilate methobromide (2.10.2),
tropenol 4,4′-difluorobenzilate methobromide (2.10.3),
scopine 4,4′-difluorobenzilate methobromide (2.10.4),
tropenol 3,3′-difluorobenzilate methobromide (2.10.5),
scopine 3,3′-difluorobenzilate methobromide (2.10.6).

The compounds of formula 2.10 may optionally be presentin the form of the enantiomers, mixtures of enantiomers or racemates thereof, as well as optionally in the form of the hydrates and/or solvates thereof.

In another preferred embodiment of the present invention the anticholinergics 2 contained in the medicament combinations according to the invention are selected from the compounds of formula 2.11 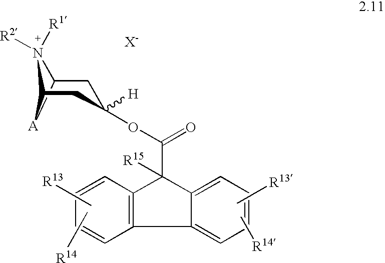
wherein
A and X⁻ may have the meanings given above and wherein
R¹⁵ denotes hydrogen, hydroxy, methyl, ethyl, —CF₃, CHF₂ or fluorine;
R^{1′} and R^{2′} which may be identical or different, denote C₁-C₅-alkyl, which may optionally be substituted by C₃-C₆-cycloalkyl, hydroxy or halogen,
   or
   R^{1′} and R^{2′} together denote a —C₃-C₅-alkylene bridge;
R¹³, R¹⁴, R^{13′} and R^{14′} which may be identical or different, denote hydrogen, —C₁-C₄-alkyl,
   —C₁-C₄-alkyloxy, hydroxy, —CF₃, —CHF₂, CN, NO₂ or halogen.

The compounds of formula 2.11 are known in the art (WO 03/064419).

Within the scope of the medicament combinations according to the invention preferred compounds of formula 2.11 are those wherein
A denotes a double-bonded group selected from 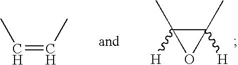
X⁻ denotes an anion selected from chloride, bromide and methanesulphonate, preferably bromide;
R¹⁵ denotes hydroxy, methyl or fluorine, preferably methyl or hydroxy;
R^{1′} and R^{2′} which may be identical or different, denote methyl or ethyl, preferably methyl;
R¹³, R¹⁴, R^{13′} and R^{14′} which may be identical or different, denote hydrogen, —CF₃, —CHF₂ or fluorine, preferably hydrogen or fluorine.

Within the scope of the medicament combinations according to the invention particularly preferred compounds of formula 2.11 are those wherein
A denotes a double-bonded group selected from 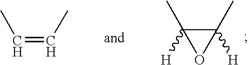
X⁻ denotes bromide;
R¹⁵ denotes hydroxy or methyl, preferably methyl; R^{1′} and R^{2′} which may be identical or different, denote methyl or ethyl, preferably methyl;
R¹³, R¹⁴, R^{13′} and R^{14′} which may be identical or different, denote hydrogen or fluorine.

Of particular importance are those medicament combinations which contain in addition to a compound of formula 1 one of the following compounds of formula 2.11:
tropenol 9-hydroxy-fluorene-9-carboxylate methobromide (2.11.1);
tropenol 9-fluoro-fluorene-9-carboxylate methobromide (2.11.2);
scopine 9-hydroxy-fluorene-9-carboxylate methobromide (2.11.3);
scopine 9-fluoro-fluorene-9-carboxylate methobromide (2.11.4);
tropenol 9-methyl-fluorene-9-carboxylate methobromide (2.11.5);
scopine 9-methyl-fluorene-9-carboxylate methobromide (2.11.6);

The compounds of formula 2.11 may optionally be presentin the form of the enantiomers, mixtures of enantiomers or racemates thereof, as well as optionally in the form of the hydrates and/or solvates thereof.

In another preferred embodiment of the present invention the anticholinergics 2 contained in the medicament combinations according to the invention are selected from the compounds of formula 2.12 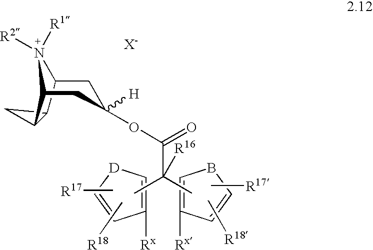
wherein X⁻ may have the meanings given above and wherein
D and B which may be identical or different, are preferably identical and denote O, S, NH, CH₂, CH═CH or
   N(C₁-C₄-alkyl);
R¹⁶ denotes hydrogen, hydroxy, —C₁-C₄-alkyl, —C₁-C₄-alkyloxy, —C₁-C₄-alkylene-halogen, —O—C₁-C₄-alkylene-halogen, —C₁-C₄-alkylene-OH, —CF₃, CHF₂, —C₁-C₄-alkylene-C₁-C₄-alkyloxy, —O—COC₁-C₄-alkyl, —O—COC₁-C₄-alkylene-halogen, —C₁-C₄-alkylene-C₃-C₆-cycloalkyl, —O—COCF₃ or halogen;
R^{1″} and R^{2″} which may be identical or different, denote —C₁-C₅-alkyl, which may optionally be substituted by —C₃-C₆-cycloalkyl, hydroxy or halogen,
   or
   R^{1″} and R^{2″} together denote a —C₃-C₅-alkylene bridge;
R¹⁷, R¹⁸, R^{17′} and R^{18′}, which may be identical or different, denote hydrogen, —C₁-C₄-alkyl, —C₁-C₄-alkyloxy, hydroxy, —CF₃, —CHF₂, CN, NO₂ or halogen;
R^{X} and R^{X′} which may be identical or different, denote hydrogen, —C₁-C₄-alkyl, —C₁-C₄-alkyloxy, hydroxy, —CF₃, —CHF₂, CN, NO₂ or halogen,
   or
R^{X} and R^{X′} together denote a single bond or one of the double-bonded groups O, S, NH, CH₂, CH₂—CH₂, N(C₁-C₄-alkyl), CH(C₁-C₄-alkyl) and —C(C₁-C₄-alkyl)₂.

The compounds of formula 2.12 are known in the art (WO 03/064418).

Within the scope of the medicament combinations according to the invention preferred compounds of formula 2.12 are those wherein
X⁻ denotes chloride, bromide or methanesulphonate, preferably bromide;
D and B which may be identical or different, are preferably identical and denote O, S, NH or CH═CH;
R¹⁶ denotes hydrogen, hydroxy, —C₁-C₄-alkyl, —C₁-C₄-alkyloxy, —CF₃, —CHF₂,
   fluorine, chlorine or bromine;
R^{1″} and R^{2″} which may be identical or different, denote C₁-C₄-alkyl, which may optionally be substituted by hydroxy, fluorine, chlorine or bromine,
   or
R^{1″} and R^{2″} together denote a —C₃-C₄-alkylene bridge;
R¹⁷, R¹⁸, R^{17′} and R¹⁸″, which may be identical or different, denote hydrogen, C₁-C₄-alkyl, C₁-C₄-alkyloxy, hydroxy, —CF₃, —CHF₂, CN, NO₂, fluorine, chlorine or bromine;
R^{X} and R^{X′} which may be identical or different, denote hydrogen, C₁-C₄-alkyl, C₁-C₄-alkyloxy, hydroxy, —CF₃, —CHF₂, CN, NO₂, fluorine, chlorine or bromine,
   or
R^{X} and R^{X′} together denote a single bond or a double-bonded group selected from O, S, NH— and CH₂.

Within the scope of the medicament combinations according to the invention particularly preferred compounds of formula 2.12 are those wherein
X⁻ denotes chloride, bromide, or methanesulphonate, preferably bromide;
D and B which may be identical or different, preferably identical, denote S or CH═CH;
R¹⁶ denotes hydrogen, hydroxy or methyl;
R^{1″} and R^{2″} which may be identical or different, denote methyl or ethyl;
R¹⁷, R¹⁸, R^{17′} and R^{18′}, which may be identical or different, denote hydrogen, —CF₃ or fluorine, preferably hydrogen;
R^{X} and R^{X′} which may be identical or different, denote hydrogen, —CF₃ or fluorine, preferably hydrogen, or
R^{X} and R^{X}′ together denote a single bond or —O.

Within the scope of the medicament combinations according to the invention, other particularly preferred compounds of formula 2.12 are those wherein
X⁻ denotes bromide;
D and B denotes —CH═CH—;
R¹⁶ denotes hydrogen, hydroxy or methyl;
R^{1″} and R^{2″} denotes methyl;
R¹⁷, R¹⁸, R^{17′} and R^{18′}, which may be identical or different, denote hydrogen or fluorine, preferably hydrogen;
R^{X} and R^{X′} which may be identical or different, denote hydrogen or fluorine, preferably hydrogen, or
R^{X} and R^{X′} together denote a single bond or the group —O.

Of particular importance are those medicament combinations which contain in addition to a compound of formula 1 one of the following compounds of formula 2.12:
cyclopropyltropine benzilate methobromide (2.12.1);
cyclopropyltropine 2,2-diphenylpropionate methobromide (2.12.2);
cyclopropyltropine 9-hydroxy-xanthene-9-carboxylate methobromide (2.12.3);
cyclopropyltropine 9-methyl-fluorene-9-carboxylate methobromide (2.12.4);
cyclopropyltropine 9-methyl-xanthene-9-carboxylate methobromide (2.12.5);
cyclopropyltropine 9-hydroxy-fluorene-9-carboxylate methobromide (2.12.6);
cyclopropyltropine methyl 4,4′-difluorobenzilate methobromide (2.12.7).

The compounds of formula 2.12 may optionally be present in the form of the enantiomers, mixtures of enantiomers or racemates thereof, as well as optionally in the form of the hydrates and/or solvates thereof.

In another preferred embodiment of the present invention the anticholinergics 2 contained in the medicament combinations according to the invention are selected from the compounds of formula 2.13 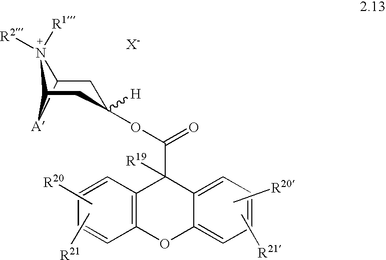
wherein X⁻ may have the meanings given above and wherein
A′ denotes a double-bonded group selected from 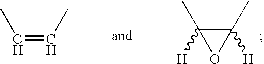
R¹⁹ denotes hydroxy, methyl, hydroxymethyl, ethyl, —CF₃, CHF₂ or fluorine;
R^{1′″} and R^{2′″} which may be identical or different, denote C₁-C₅-alkyl, which may optionally be substituted by C₃-C₆-cycloalkyl, hydroxy or halogen,
   or
R^{1′″} and R^{2′″} together denote a —C₃-C₅-alkylene bridge;
R²⁰, R²¹, R^{20′} and R^{21′} which may be identical or different, denote hydrogen, —C₁-C₄-alkyl, —C₁-C₄-alkyloxy, hydroxy, —CF₃, —CHF₂, CN, NO₂ or halogen.

The compounds of formula 2.13 are known in the art (WO 03/064417).

Within the scope of the medicament combinations according to the invention preferred compounds of formula 2.13 are those wherein
A′ denotes a double-bonded group selected from 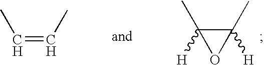
X⁻ denotes chloride, bromide or methanesulphnat, preferably bromide;
R¹⁹ denotes hydroxy or methyl;
R^{1′″} and R^{2′″} which may be identical or different, denote methyl or ethyl, preferably methyl;
R²⁰, R²¹, R^{20′} and R^{21′} which may be identical or different, denote hydrogen, —CF₃, —CHF₂ or fluorine, preferably hydrogen or fluorine.

Within the scope of the medicament combinations according to the invention particularly preferred compounds of formula 2.13 are those wherein
A′ denotes a double-bonded group selected from 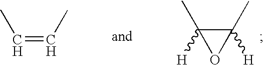
X⁻ denotes bromide;
R¹⁹ denotes hydroxy or methyl, preferably methyl;
R^{1′″} and R^{2′″} which may be identical or different, denote methyl or ethyl, preferably methyl;
R³, R⁴, R^{3′} and R^{4′} which may be identical or different, denote hydrogen or fluorine.

Of particular importance are those medicament combinations which contain in addition to a compound of formula 1 one of the following compounds of formula 2.13:
tropenol 9-hydroxy-xanthene-9-carboxylate methobromide (2.13.1);
scopine 9-hydroxy-xanthene-9-carboxylate methobromide (2.13.2);
tropenol 9-methyl-xanthene-9-carboxylate methobromide (2.13.3);
scopine 9-methyl-xanthene-9-carboxylate methobromide (2.13.4);
tropenol 9-ethyl-xanthene-9-carboxylate methobromide (2.13.5);
tropenol 9-difluoromethyl-xanthene-9-carboxylate methobromide (2.13.6);
scopine 9-hydroxymethyl-xanthene-9-carboxylate methobromide (2.13.7).

The compounds of formula 2.13 may optionally be present in the form of the enantiomers, mixtures of enantiomers or racemates thereof, as well as optionally in the form of the hydrates and/or solvates thereof.

Within the scope of the present invention any reference to anticholinergics 2′ is to be taken as a reference to the pharmacologically active cations of the various salts. These cations are tiotropium (2.1′), oxitropium (2.2′), flutropium (2.3′), ipratropium (2.4′), glycopyrronium (2.5′), trospium (2.6′) and the cations shown below:

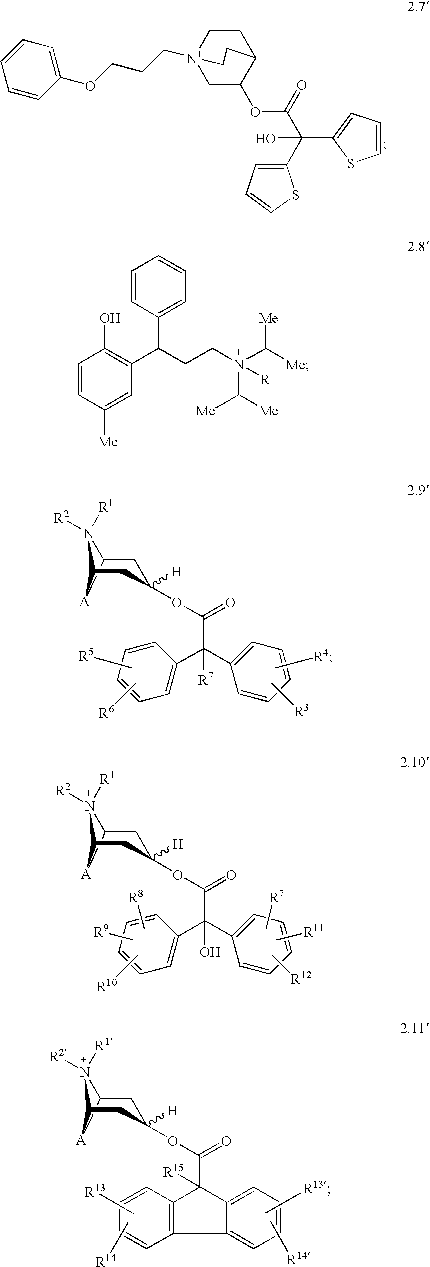

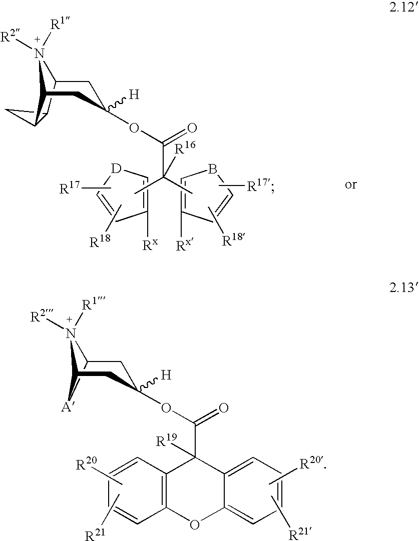

In the medicament combinations according to the invention the PDE IV-inhibitor 3 is preferably selected from among enprofyllin (3.1), theophyllin (3.2), roflumilast (3.3), ariflo (Cilomilast, 3.4)), CP-325,366 (3.5), BY343 (3.6), D-4396 (Sch-351591, 3.7)), AWD-12-281 (GW-842470, 3.8)), N-(3,5-dichloro-1-oxo-pyridin-4-yl)-4-difluoromethoxy-3-cyclopropylmethoxybenzamide (3.9), NCS-613 (3.10), pumafentine (3.11), (−)p-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamide (3.12), (R)-(+)-1-(4-bromobenzyl)-4-[(3-cyclopentyloxy)-4-methoxyphenyl]-2-pyrrolidone (3.13), 3-(cyclopentyloxy-4-methoxyphenyl)-1-(4-N′-[N-2-cyano-S-methyl-isothioureido]benzyl)-2-pyrrolidone (3.14), cis[4-cyano-4-(3-cyclopentyloxy-4-methoxyphenyl)cyclohexane-1-carboxylic acid] (3.15), 2-carbomethoxy-4-cyano-4-(3-cyclopropylmethoxy-4-difluoromethoxyphenyl)cyclohexan-1-one (3.16), cis[4-cyano-4-(3-cyclopropylmethoxy-4-difluoromethoxyphenyl)cyclohexan-1-ol] (3.17), (R)-(+)-ethyl[4-(3-cyclopentyloxy-4-methoxyphenyl)pyrrolidin-2-ylidene]acetate (3.18), (S)-(−)-ethyl[4-(3-cyclopentyloxy-4-methoxyphenyl)pyrrolidin-2-ylidene]acetate (3.19), 4-(3-cyclopentyloxy-4-methoxyphenyl)-3-(1-hydroxy-ethyl)-3-methyl-pyrrolidine-1-carboxylic acid methyl ester (=IC 485, 3.20), CDP840 (3.21), Bay-198004 (3.22), D-4418 (3.23), PD-168787 (3.24), T-440 (3.25), T-2585 (3.26), arofyllin (3.27), atizoram (3.28), V-11294A (3.29), C1-1018 (3.30), CDC-801 (3.31), CDC-3052 (3.32), D-22888 (3.33), YM-58997 (3.34), Z-15370 (3.35), 9-cyclopentyl-5,6-dihydro-7-ethyl-3-(2-thienyl)-9H-pyrazolo[3,4-c]-1,2,4-triazolo[4,3-a]pyridine (3.36), 9-cyclopentyl-5,6-dihydro-7-ethyl-3-(tert-butyl)-9H-pyrazolo[3,4-c]-1,2,4-triazolo[4,3-a]pyridine (3.37), and tetomilast (3.38), optionally in the form of the racemates, enantiomers or diastereomers thereof and optionally in the form of the pharmacologically acceptable acid addition salts, solvates and/or hydrates thereof.

In particularly preferred medicament combinations the PDE IV-inhibitor 3 is selected from the group comprising enprofyllin (3.1), roflumilast (3.3) optionally also in form of the roflumilast N-oxide, ariflo (cilomilast) (3.4), AWD-12-281 (GW-842470) (3.8), N-(3,5-dichloro-1-oxo-pyridin-4-yl)-4-difluoromethoxy-3-cyclopropylmethoxybenzamide (3.9), T-440 (3.25), T-2585 (3.26), cis[4-cyano-4-(3-cyclopentyloxy-4-methoxyphenyl)cyclohexane-1-carboxylic acid] (3.15), 2-carbomethoxy-4-cyano-4-(3-cyclopropylmethoxy-4-difluoromethoxyphenyl)cyclohexan-1-one (3.16), cis[4-cyano-4-(3-cyclopropylmethoxy-4-difluoromethoxyphenyl)cyclohexan-1-ol] (3.17), 4-(3-cyclopentyloxy-4-methoxy-phenyl)-3-(1-hydroxy-ethyl)-3-methyl-pyrrolidine-1-carboxylic acid methyl ester (=IC 485, 3.20), PD-168787 (3.24), arofyllin (3.27), atizoram (3.28), V-11294A (3.29), C1-1018 (3.30), CDC-801 (3.31), D-22888 (3.33), YM-58997 (3.34), Z-15370 (3.35), 9-cyclopentyl-5,6-dihydro-7-ethyl-3-(2-thienyl)-9H-pyrazolo[3,4-c]-1,2,4-triazolo[4,3-a]pyridine (3.36), 9-cyclopentyl-5,6-dihydro-7-ethyl-3-(tert-butyl)-9H-pyrazolo[3,4-c]-1,2,4-triazolo[4,3-a]pyridine (3.37), and tetomilast (3.38), optionally in the form of the racemates, enantiomers or diastereomers thereof and optionally in the form of the pharmacologically acceptable acid addition salts, solvates and/or hydrates thereof.

In particularly preferred medicament combinations the PDE IV-inhibitor 3 is selected from the group comprising roflumilast (3.3), ariflo (cilomilast) (3.4), AWD-12-281 (GW-842470) (3.8), 2-carbomethoxy-4-cyano-4-(3-cyclopropylmethoxy-4-difluoromethoxyphenyl)cyclohexan-1-one (3.16), cis[4-cyano-4-(3-cyclopropylmethoxy-4-difluoromethoxyphenyl)cyclohexan-1-ol] (3.17), 4-(3-cyclopentyloxy-4-methoxyphenyl)-3-(1-hydroxy-ethyl)-3-methyl-pyrrolidine-1-carboxylic acid methyl ester (=IC 485, 3.20), arofyllin (3.27), atizoram (3.28), Z-15370 (3.35), 9-cyclopentyl-5,6-dihydro-7-ethyl-3-(2-thienyl)-9H-pyrazolo[3,4-c]-1,2,4-triazolo[4,3-a]pyridine (3.36), 9-cyclopentyl-5,6-dihydro-7-ethyl-3-(tert-butyl)-9H-pyrazolo[3,4-c]-1,2,4-triazolo[4,3-a]pyridine (3.37), and tetomilast (3.38), while roflumilast (3.3), Z-15370 (3.35) and AWD-12-281 (3.8) are of particular significance, optionally in the form of the racemates, enantiomers or diastereomers thereof and optionally in the form of the pharmacologically acceptable acid addition salts, solvates and/or hydrates thereof.

By the acid addition salts with pharmacologically acceptable acids which the compounds 3 may possibly be capable of forming are meant for example salts selected from the group comprising the hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydrooxalate, hydrosuccinate, hydrobenzoate and hydro-p-toluenesulphonate, preferably the hydrochloride, hydrobromide, hydrosulphate, hydrophosphate, hydrofumarate and hydromethanesulphonate.

Examples of novel preferred medicament combinations of preferred betamimetics 1 with anticholinergis 2 and PDE IV inhibitors 3 include the following exemplified combinations:

1.7, 2.1 and 3.1; 1.7, 2.2 and 3.1; 1.7, 2.3 and 3.1; 1.7, 2.1 and 3.3; 1.7, 2.2 and 3.3; 1.7, 2.3 and 3.3; 1.7, 2.1 and 3.4; 1.7, 2.2 and 3.4; 1.7, 2.3 and 3.4; 1.7, 2.1 and 3.8; 1.7, 2.2 and 3.8; 1.7, 2.3 and 3.8; 1.7, 2.1 and 3.9; 1.7, 2.2 and 3.9; 1.7, 2.3 and 3.9; 1.7, 2.1 and 3.16; 1.7, 2.2 and 3.16; 1.7, 2.3 and 3.16; 1.7, 2.1 and 3.17; 1.7, 2.2 and 3.17; 1.7, 2.3 and 3.17;

1.7, 2.1 and 3.20; 1.7, 2.2 and 3.20; 1.7, 2.3 and 3.20; 1.7, 2.1 and 3.24; 1.7, 2.2 and 3.24; 1.7, 2.3 and 3.24; 1.7, 2.1 and 3.25; 1.7, 2.2 and 3.25; 1.7, 2.3 and 3.25; 1.7, 2.1 and 3.26; 1.7, 2.2 and 3.26; 1.7, 2.3 and 3.26; 1.7, 2.1 and 3.27; 1.7, 2.2 and 3.27; 1.7, 2.3 and 3.27; 1.7, 2.1 and 3.28; 1.7, 2.2 and 3.28; 1.7, 2.3 and 3.28; 1.7, 2.1 and 3.29; 1.7, 2.2 and 3.29; 1.7, 2.3 and 3.29; 1.7, 2.1 and 3.30; 1.7, 2.2 and 3.30; 1.7, 2.3 and 3.30; 1.7, 2.1 and 3.31; 1.7, 2.2 and 3.31; 1.7, 2.3 and 3.31; 1.7, 2.1 and 3.33; 1.7, 2.2 and 3.33; 1.7, 2.3 and 3.33; 1.7, 2.1 and 3.34; 1.7, 2.2 and 3.34; 1.7, 2.3 and 3.34; 1.7, 2.1 and 3.35; 1.7, 2.2 and 3.35; 1.7, 2.3 and 3.35; 1.7, 2.1 and 3.36; 1.7, 2.2 and 3.36; 1.7, 2.3 and 3.36; 1.7, 2.1 and 3.37; 1.7, 2.1 and 3.38; 1.7, 2.2 and 3.37; 1.7, 2.2 and 3.38; 1.7, 2.3 and 3.37; 1.7, 2.3 and 3.38; 1.7, 2.4 and 3.1; 1.7, 2.5 and 3.1; 1.7, 2.7 and 3.1; 1.7, 2.4 and 3.3; 1.7, 2.5 and 3.3; 1.7, 2.7 and 3.3; 1.7, 2.4 and 3.4; 1.7, 2.5 and 3.4; 1.7, 2.7 and 3.4; 1.7, 2.4 and 3.8; 1.7, 2.5 and 3.8; 1.7, 2.7 and 3.8; 1.7, 2.4 and 3.9; 1.7, 2.5 and 3.9; 1.7, 2.7 and 3.9; 1.7, 2.4 and 3.16; 1.7, 2.5 and 3.16; 1.7, 2.7 and 3.16; 1.7, 2.4 and 3.17; 1.7, 2.5 and 3.17; 1.7, 2.7 and 3.17; 1.7, 2.4 and 3.20; 1.7, 2.5 and 3.20; 1.7, 2.7 and 3.20; 1.7, 2.4 and 3.24; 1.7, 2.5 and 3.24; 1.7, 2.7 and 3.24; 1.7, 2.4 and 3.25; 1.7, 2.5 and 3.25; 1.7, 2.7 and 3.25; 1.7, 2.4 and 3.26; 1.7, 2.5 and 3.26; 1.7, 2.7 and 3.26; 1.7, 2.4 and 3.27; 1.7, 2.5 and 3.27; 1.7, 2.7 and 3.27; 1.7, 2.4 and 3.28; 1.7, 2.5 and 3.28; 1.7, 2.7 and 3.28; 1.7, 2.4 and 3.29; 1.7, 2.5 and 3.29; 1.7, 2.7 and 3.29; 1.7, 2.4 and 3.30; 1.7, 2.5 and 3.30; 1.7, 2.7 and 3.30; 1.7, 2.4 and 3.31; 1.7, 2.5 and 3.31; 1.7, 2.7 and 3.31; 1.7, 2.4 and 3.33; 1.7, 2.5 and 3.33; 1.7, 2.7 and 3.33; 1.7, 2.4 and 3.34; 1.7, 2.5 and 3.34; 1.7, 2.7 and 3.34; 1.7, 2.4 and 3.35; 1.7, 2.5 and 3.35; 1.7, 2.7 and 3.35; 1.7, 2.4 and 3.36; 1.7, 2.5 and 3.36; 1.7, 2.7 and 3.36; 1.7, 2.4 and 3.37; 1.7, 2.4 and 3.38; 1.7, 2.5 and 3.37; 1.7, 2.5 and 3.38; 1.7, 2.7 and 3.37; 1.7, 2.7 and 3.38; 1.7, 2.9.1 and 3.1; 1.7, 2.9.2 and 3.1; 1.7, 2.9.1 and 3.3; 1.7, 2.9.2 and 3.3; 1.7, 2.9.1 and 3.4; 1.7, 2.9.2 and 3.4; 1.7, 2.9.1 and 3.8; 1.7, 2.9.2 and 3.8; 1.7, 2.9.1 and 3.9; 1.7, 2.9.2 and 3.9; 1.7, 2.9.1 and 3.16; 1.7, 2.9.2 and 3.16; 1.7, 2.9.1 and 3.17; 1.7, 2.9.2 and 3.17; 1.7, 2.9.1 and 3.20; 1.7, 2.9.2 and 3.20; 1.7, 2.9.1 and 3.24; 1.7, 2.9.2 and 3.24; 1.7, 2.9.1 and 3.25; 1.7, 2.9.2 and 3.25; 1.7, 2.9.1 and 3.26; 1.7, 2.9.2 and 3.26; 1.7, 2.9.1 and 3.27; 1.7, 2.9.2 and 3.27; 1.7, 2.9.1 and 3.28; 1.7, 2.9.2 and 3.28; 1.7, 2.9.1 and 3.29; 1.7, 2.9.2 and 3.29; 1.7, 2.9.1 and 3.30; 1.7, 2.9.2 and 3.30; 1.7, 2.9.1 and 3.31; 1.7, 2.9.2 and 3.31; 1.7, 2.9.1 and 3.33; 1.7, 2.9.2 and 3.33; 1.7, 2.9.1 and 3.34; 1.7, 2.9.2 and 3.34; 1.7, 2.9.1 and 3.35; 1.7, 2.9.2 and 3.35; 1.7, 2.9.1 and 3.36; 1.7, 2.9.2 and 3.36; 1.7, 2.9.1 and 3.37; 1.7, 2.9.1 and 3.38; 1.7, 2.9.2 and 3.37; 1.7, 2.9.2 and 3.38; 1.7, 2.10.1 and 3.1; 1.7, 2.10.2 and 3.1; 1.7, 2.10.1 and 3.3; 1.7, 2.10.2 and 3.3; 1.7, 2.10.1 and 3.4; 1.7, 2.10.2 and 3.4; 1.7, 2.10.1 and 3.8; 1.7, 2.10.2 and 3.8; 1.7, 2.10.1 and 3.9; 1.7, 2.10.2 and 3.9; 1.7, 2.10.1 and 3.16; 1.7, 2.10.2 and 3.16; 1.7, 2.10.1 and 3.17; 1.7, 2.10.2 and 3.17; 1.7, 2.10.1 and 3.20; 1.7, 2.10.2 and 3.20; 1.7, 2.10.1 and 3.24; 1.7, 2.10.2 and 3.24; 1.7, 2.10.1 and 3.25; 1.7, 2.10.2 and 3.25; 1.7, 2.10.1 and 3.26; 1.7, 2.10.2 and 3.26; 1.7, 2.10.1 and 3.27; 1.7, 2.10.2 and 3.27; 1.7, 2.10.1 and 3.28; 1.7, 2.10.2 and 3.28; 1.7, 2.10.1 and 3.29; 1.7, 2.10.2 and 3.29; 1.7, 2.10.1 and 3.30; 1.7, 2.10.2 and 3.30; 1.7, 2.10.1 and 3.31; 1.7, 2.10.2 and 3.31; 1.7, 2.10.1 and 3.33; 1.7, 2.10.2 and 3.33; 1.7, 2.10.1 and 3.34; 1.7, 2.10.2 and 3.34; 1.7, 2.10.1 and 3.35; 1.7, 2.10.2 and 3.35; 1.7, 2.10.1 and 3.36; 1.7, 2.10.2 and 3.36; 1.7, 2.10.1 and 3.37; 1.7, 2.10.1 and 3.38; 1.7, 2.10.2 and 3.37; 1.7, 2.10.2 and 3.38; 1.7, 2.11.5 and 3.1; 1.7, 2.11.5 and 3.3; 1.7, 2.11.5 and 3.4; 1.7, 2.11.5 and 3.8; 1.7, 2.11.5 and 3.9; 1.7, 2.11.5 and 3.16; 1.7, 2.11.5 and 3.17; 1.7, 2.11.5 and 3.20; 1.7, 2.11.5 and 3.24; 1.7, 2.11.5 and 3.25; 1.7, 2.11.5 and 3.26; 1.7, 2.11.5 and 3.27; 1.7, 2.11.5 and 3.28; 1.7, 2.11.5 and 3.29; 1.7, 2.11.5 and 3.30; 1.7, 2.11.5 and 3.31; 1.7, 2.11.5 and 3.33; 1.7, 2.11.5 and 3.34; 1.7, 2.11.5 and 3.35; 1.7, 2.11.5 and 3.36; 1.7, 2.11.5 and 3.37; 1.7, 2.11.5 and 3.38; 1.7, 2.11.6 and 3.1; 1.7, 2.12.1 and 3.1; 1.7, 2.12.2 and 3.1; 1.7, 2.11.6 and 3.3; 1.7, 2.12.1 and 3.3; 1.7, 2.12.2 and 3.3; 1.7, 2.11.6 and 3.4; 1.7, 2.12.1 and 3.4; 1.7, 2.12.2 and 3.4; 1.7, 2.11.6 and 3.8; 1.7, 2.12.1 and 3.8; 1.7, 2.12.2 and 3.8; 1.7, 2.11.6 and 3.9; 1.7, 2.12.1 and 3.9; 1.7, 2.12.2 and 3.9; 1.7, 2.11.6 and 3.16; 1.7, 2.12.1 and 3.16; 1.7, 2.12.2 and 3.16; 1.7, 2.11.6 and 3.17; 1.7, 2.12.1 and 3.17; 1.7, 2.12.2 and 3.17; 1.7, 2.11.6 and 3.20; 1.7, 2.12.1 and 3.20; 1.7, 2.12.2 and 3.20; 1.7, 2.11.6 and 3.24; 1.7, 2.12.1 and 3.24; 1.7, 2.12.2 and 3.24; 1.7, 2.11.6 and 3.25; 1.7, 2.12.1 and 3.25; 1.7, 2.12.2 and 3.25; 1.7, 2.11.6 and 3.26; 1.7, 2.12.1 and 3.26; 1.7, 2.12.2 and 3.26; 1.7, 2.11.6 and 3.27; 1.7, 2.12.1 and 3.27; 1.7, 2.12.2 and 3.27; 1.7, 2.11.6 and 3.28; 1.7, 2.12.1 and 3.28; 1.7, 2.12.2 and 3.28; 1.7, 2.11.6 and 3.29; 1.7, 2.12.1 and 3.29; 1.7, 2.12.2 and 3.29; 1.7, 2.11.6 and 3.30; 1.7, 2.12.1 and 3.30; 1.7, 2.12.2 and 3.30; 1.7, 2.11.6 and 3.31; 1.7, 2.12.1 and 3.31; 1.7, 2.12.2 and 3.31; 1.7, 2.11.6 and 3.33; 1.7, 2.12.1 and 3.33; 1.7, 2.12.2 and 3.33; 1.7, 2.11.6 and 3.34; 1.7, 2.12.1 and 3.34; 1.7, 2.12.2 and 3.34; 1.7, 2.11.6 and 3.35; 1.7, 2.12.1 and 3.35; 1.7, 2.12.2 and 3.35; 1.7, 2.11.6 and 3.36; 1.7, 2.12.1 and 3.36; 1.7, 2.12.2 and 3.36; 1.7, 2.11.6 and 3.37; 1.7, 2.11.6 and 3.38; 1.7, 2.12.1 and 3.37; 1.7, 2.12.1 and 3.38; 1.7, 2.12.2 and 3.37; 1.7, 2.12.2 and 3.38; 1.7, 2.13.3 and 3.1; 1.7, 2.13.4 and 3.1; 1.7, 2.13.3 and 3.3; 1.7, 2.13.4 and 3.3; 1.7, 2.13.3 and 3.4; 1.7, 2.13.4 and 3.4; 1.7, 2.13.3 and 3.8; 1.7, 2.13.4 and 3.8; 1.7, 2.13.3 and 3.9; 1.7, 2.13.4 and 3.9; 1.7, 2.13.3 and 3.16; 1.7, 2.13.4 and 3.16; 1.7, 2.13.3 and 3.17; 1.7, 2.13.4 and 3.17; 1.7, 2.13.3 and 3.20; 1.7, 2.13.4 and 3.20; 1.7, 2.13.3 and 3.24; 1.7, 2.13.4 and 3.24; 1.7, 2.13.3 and 3.25; 1.7, 2.13.4 and 3.25; 1.7, 2.13.3 and 3.26; 1.7, 2.13.4 and 3.26; 1.7, 2.13.3 and 3.27; 1.7, 2.13.4 and 3.27; 1.7, 2.13.3 and 3.28; 1.7, 2.13.4 and 3.28; 1.7, 2.13.3 and 3.29; 1.7, 2.13.4 and 3.29; 1.7, 2.13.3 and 3.30; 1.7, 2.13.4 and 3.30; 1.7, 2.13.3 and 3.31; 1.7, 2.13.4 and 3.31; 1.7, 2.13.3 and 3.33; 1.7, 2.13.4 and 3.33; 1.7, 2.13.3 and 3.34; 1.7, 2.13.4 and 3.34; 1.7, 2.13.3 and 3.35; 1.7, 2.13.4 and 3.35; 1.7, 2.13.3 and 3.36; 1.7, 2.13.4 and 3.36; 1.7, 2.13.3 and 3.37; 1.7, 2.13.3 and 3.38; 1.7, 2.13.4 and 3.37; 1.7, 2.13.4 and 3.38; 1.8, 2.1 and 3.1; 1.8, 2.2 and 3.1; 1.8, 2.3 and 3.1; 1.8, 2.1 and 3.3; 1.8, 2.2 and 3.3; 1.8, 2.3 and 3.3; 1.8, 2.1 and 3.4; 1.8, 2.2 and 3.4; 1.8, 2.3 and 3.4; 1.8, 2.1 and 3.8; 1.8, 2.2 and 3.8; 1.8, 2.3 and 3.8; 1.8, 2.1 and 3.9; 1.8, 2.2 and 3.9; 1.8, 2.3 and 3.9; 1.8, 2.1 and 3.16; 1.8, 2.2 and 3.16; 1.8, 2.3 and 3.16; 1.8, 2.1 and 3.17; 1.8, 2.2 and 3.17; 1.8, 2.3 and 3.17; 1.8, 2.1 and 3.20; 1.8, 2.2 and 3.20; 1.8, 2.3 and 3.20; 1.8, 2.1 and 3.24; 1.8, 2.2 and 3.24; 1.8, 2.3 and 3.24; 1.8, 2.1 and 3.25; 1.8, 2.2 and 3.25; 1.8, 2.3 and 3.25; 1.8, 2.1 and 3.26; 1.8, 2.2 and 3.26; 1.8, 2.3 and 3.26; 1.8, 2.1 and 3.27; 1.8, 2.2 and 3.27; 1.8, 2.3 and 3.27; 1.8, 2.1 and 3.28; 1.8, 2.2 and 3.28; 1.8, 2.3 and 3.28; 1.8, 2.1 and 3.29; 1.8, 2.2 and 3.29; 1.8, 2.3 and 3.29; 1.8, 2.1 and 3.30; 1.8, 2.2 and 3.30; 1.8, 2.3 and 3.30; 1.8, 2.1 and 3.31; 1.8, 2.2 and 3.31; 1.8, 2.3 and 3.31; 1.8, 2.1 and 3.33; 1.8, 2.2 and 3.33; 1.8, 2.3 and 3.33; 1.8, 2.1 and 3.34; 1.8, 2.2 and 3.34; 1.8, 2.3 and 3.34; 1.8, 2.1 and 3.35; 1.8, 2.2 and 3.35; 1.8, 2.3 and 3.35; 1.8, 2.1 and 3.36; 1.8, 2.2 and 3.36; 1.8, 2.3 and 3.36; 1.8, 2.1 and 3.37; 1.8, 2.1 and 3.38; 1.8, 2.2 and 3.37; 1.8, 2.2 and 3.38; 1.8, 2.3 and 3.37; 1.8, 2.3 and 3.38; 1.8, 2.4 and 3.1; 1.8, 2.5 and 3.1; 1.8, 2.7 and 3.1; 1.8, 2.4 and 3.3; 1.8, 2.5 and 3.3; 1.8, 2.7 and 3.3; 1.8, 2.4 and 3.4; 1.8, 2.5 and 3.4; 1.8, 2.7 and 3.4; 1.8, 2.4 and 3.8; 1.8, 2.5 and 3.8; 1.8, 2.7 and 3.8; 1.8, 2.4 and 3.9; 1.8, 2.5 and 3.9; 1.8, 2.7 and 3.9; 1.8, 2.4 and 3.16; 1.8, 2.5 and 3.16; 1.8, 2.7 and 3.16; 1.8, 2.4 and 3.17; 1.8, 2.5 and 3.17; 1.8, 2.7 and 3.17; 1.8, 2.4 and 3.20; 1.8, 2.5 and 3.20; 1.8, 2.7 and 3.20; 1.8, 2.4 and 3.24; 1.8, 2.5 and 3.24; 1.8, 2.7 and 3.24; 1.8, 2.4 and 3.25; 1.8, 2.5 and 3.25; 1.8, 2.7 and 3.25; 1.8, 2.4 and 3.26; 1.8, 2.5 and 3.26; 1.8, 2.7 and 3.26; 1.8, 2.4 and 3.27; 1.8, 2.5 and 3.27; 1.8, 2.7 and 3.27; 1.8, 2.4 and 3.28; 1.8, 2.5 and 3.28; 1.8, 2.7 and 3.28; 1.8, 2.4 and 3.29; 1.8, 2.5 and 3.29; 1.8, 2.7 and 3.29; 1.8, 2.4 and 3.30; 1.8, 2.5 and 3.30; 1.8, 2.7 and 3.30; 1.8, 2.4 and 3.31; 1.8, 2.5 and 3.31; 1.8, 2.7 and 3.31; 1.8, 2.4 and 3.33; 1.8, 2.5 and 3.33; 1.8, 2.7 and 3.33; 1.8, 2.4 and 3.34; 1.8, 2.5 and 3.34; 1.8, 2.7 and 3.34; 1.8, 2.4 and 3.35; 1.8, 2.5 and 3.35; 1.8, 2.7 and 3.35; 1.8, 2.4 and 3.36; 1.8, 2.5 and 3.36; 1.8, 2.7 and 3.36; 1.8, 2.4 and 3.37; 1.8, 2.4 and 3.38; 1.8, 2.5 and 3.37; 1.8, 2.5 and 3.38; 1.8, 2.7 and 3.37; 1.8, 2.7 and 3.38; 1.8, 2.9.1 and 3.1; 1.8, 2.9.2 and 3.1; 1.8, 2.9.1 and 3.3; 1.8, 2.9.2 and 3.3; 1.8, 2.9.1 and 3.4; 1.8, 2.9.2 and 3.4; 1.8, 2.9.1 and 3.8; 1.8, 2.9.2 and 3.8; 1.8, 2.9.1 and 3.9; 1.8, 2.9.2 and 3.9; 1.8, 2.9.1 and 3.16; 1.8, 2.9.2 and 3.16; 1.8, 2.9.1 and 3.17; 1.8, 2.9.2 and 3.17; 1.8, 2.9.1 and 3.20; 1.8, 2.9.2 and 3.20; 1.8, 2.9.1 and 3.24; 1.8, 2.9.2 and 3.24; 1.8, 2.9.1 and 3.25; 1.8, 2.9.2 and 3.25; 1.8, 2.9.1 and 3.26; 1.8, 2.9.2 and 3.26; 1.8, 2.9.1 and 3.27; 1.8, 2.9.2 and 3.27; 1.8, 2.9.1 and 3.28; 1.8, 2.9.2 and 3.28; 1.8, 2.9.1 and 3.29; 1.8, 2.9.2 and 3.29; 1.8, 2.9.1 and 3.30; 1.8, 2.9.2 and 3.30; 1.8, 2.9.1 and 3.31; 1.8, 2.9.2 and 3.31; 1.8, 2.9.1 and 3.33; 1.8, 2.9.2 and 3.33; 1.8, 2.9.1 and 3.34; 1.8, 2.9.2 and 3.34; 1.8, 2.9.1 and 3.35; 1.8, 2.9.2 and 3.35; 1.8, 2.9.1 and 3.36; 1.8, 2.9.2 and 3.36; 1.8, 2.9.1 and 3.37; 1.8, 2.9.1 and 3.38; 1.8, 2.9.2 and 3.37; 1.8, 2.9.2 and 3.38; 1.8, 2.10.1 and 3.1; 1.8, 2.10.2 and 3.1; 1.8, 2.10.1 and 3.3; 1.8, 2.10.2 and 3.3; 1.8, 2.10.1 and 3.4; 1.8, 2.10.2 and 3.4; 1.8, 2.10.1 and 3.8; 1.8, 2.10.2 and 3.8; 1.8, 2.10.1 and 3.9; 1.8, 2.10.2 and 3.9; 1.8, 2.10.1 and 3.16; 1.8, 2.10.2 and 3.16; 1.8, 2.10.1 and 3.17; 1.8, 2.10.2 and 3.17; 1.8, 2.10.1 and 3.20; 1.8, 2.10.2 and 3.20; 1.8, 2.10.1 and 3.24; 1.8, 2.10.2 and 3.24; 1.8, 2.10.1 and 3.25; 1.8, 2.10.2 and 3.25; 1.8, 2.10.1 and 3.26; 1.8, 2.10.2 and 3.26; 1.8, 2.10.1 and 3.27; 1.8, 2.10.2 and 3.27; 1.8, 2.10.1 and 3.28; 1.8, 2.10.2 and 3.28; 1.8, 2.10.1 and 3.29; 1.8, 2.10.2 and 3.29; 1.8, 2.10.1 and 3.30; 1.8, 2.10.2 and 3.30; 1.8, 2.10.1 and 3.31; 1.8, 2.10.2 and 3.31; 1.8, 2.10.1 and 3.33; 1.8, 2.10.2 and 3.33; 1.8, 2.10.1 and 3.34; 1.8, 2.10.2 and 3.34; 1.8, 2.10.1 and 3.35; 1.8, 2.10.2 and 3.35; 1.8, 2.10.1 and 3.36; 1.8, 2.10.2 and 3.36; 1.8, 2.10.1 and 3.37; 1.8, 2.10.1 and 3.38; 1.8, 2.10.2 and 3.37; 1.8, 2.10.2 and 3.38; 1.8, 2.11.5 and 3.1; 1.8, 2.11.5 and 3.3; 1.8, 2.11.5 and 3.4; 1.8, 2.11.5 and 3.8; 1.8, 2.11.5 and 3.9; 1.8, 2.11.5 and 3.16; 1.8, 2.11.5 and 3.17; 1.8, 2.11.5 and 3.20; 1.8, 2.11.5 and 3.24; 1.8, 2.11.5 and 3.25; 1.8, 2.11.5 and 3.26; 1.8, 2.11.5 and 3.27; 1.8, 2.11.5 and 3.28; 1.8, 2.11.5 and 3.29; 1.8, 2.11.5 and 3.30; 1.8, 2.11.5 and 3.31; 1.8, 2.11.5 and 3.33; 1.8, 2.11.5 and 3.34; 1.8, 2.11.5 and 3.35; 1.8, 2.11.5 and 3.36; 1.8, 2.11.5 and 3.37; 1.8, 2.11.5 and 3.38; 1.8, 2.11.6 and 3.1; 1.8, 2.12.1 and 3.1; 1.8, 2.12.2 and 3.1; 1.8, 2.11.6 and 3.3; 1.8, 2.12.1 and 3.3; 1.8, 2.12.2 and 3.3; 1.8, 2.11.6 and 3.4; 1.8, 2.12.1 and 3.4; 1.8, 2.12.2 and 3.4; 1.8, 2.11.6 and 3.8; 1.8, 2.12.1 and 3.8; 1.8, 2.12.2 and 3.8; 1.8, 2.11.6 and 3.9; 1.8, 2.12.1 and 3.9; 1.8, 2.12.2 and 3.9; 1.8, 2.11.6 and 3.16; 1.8, 2.12.1 and 3.16; 1.8, 2.12.2 and 3.16; 1.8, 2.11.6 and 3.17; 1.8, 2.12.1 and 3.17; 1.8, 2.12.2 and 3.17; 1.8, 2.11.6 and 3.20; 1.8, 2.12.1 and 3.20; 1.8, 2.12.2 and 3.20; 1.8, 2.11.6 and 3.24; 1.8, 2.12.1 and 3.24; 1.8, 2.12.2 and 3.24; 1.8, 2.11.6 and 3.25; 1.8, 2.12.1 and 3.25; 1.8, 2.12.2 and 3.25; 1.8, 2.11.6 and 3.26; 1.8, 2.12.1 and 3.26; 1.8, 2.12.2 and 3.26; 1.8, 2.11.6 and 3.27; 1.8, 2.12.1 and 3.27; 1.8, 2.12.2 and 3.27; 1.8, 2.11.6 and 3.28; 1.8, 2.12.1 and 3.28; 1.8, 2.12.2 and 3.28; 1.8, 2.11.6 and 3.29; 1.8, 2.12.1 and 3.29; 1.8, 2.12.2 and 3.29; 1.8, 2.11.6 and 3.30; 1.8, 2.12.1 and 3.30; 1.8, 2.12.2 and 3.30; 1.8, 2.11.6 and 3.31; 1.8, 2.12.1 and 3.31; 1.8, 2.12.2 and 3.31; 1.8, 2.11.6 and 3.33; 1.8, 2.12.1 and 3.33; 1.8, 2.12.2 and 3.33; 1.8, 2.11.6 and 3.34; 1.8, 2.12.1 and 3.34; 1.8, 2.12.2 and 3.34; 1.8, 2.11.6 and 3.35; 1.8, 2.12.1 and 3.35; 1.8, 2.12.2 and 3.35; 1.8, 2.11.6 and 3.36; 1.8, 2.12.1 and 3.36; 1.8, 2.12.2 and 3.36; 1.8, 2.11.6 and 3.37; 1.8, 2.11.6 and 3.38; 1.8, 2.12.1 and 3.37; 1.8, 2.12.1 and 3.38; 1.8, 2.12.2 and 3.37; 1.8, 2.12.2 and 3.38; 1.8, 2.13.3 and 3.1; 1.8, 2.13.4 and 3.1; 1.8, 2.13.3 and 3.3; 1.8, 2.13.4 and 3.3; 1.8, 2.13.3 and 3.4; 1.8, 2.13.4 and 3.4; 1.8, 2.13.3 and 3.8; 1.8, 2.13.4 and 3.8; 1.8, 2.13.3 and 3.9; 1.8, 2.13.4 and 3.9; 1.8, 2.13.3 and 3.16; 1.8, 2.13.4 and 3.16; 1.8, 2.13.3 and 3.17; 1.8, 2.13.4 and 3.17; 1.8, 2.13.3 and 3.20; 1.8, 2.13.4 and 3.20; 1.8, 2.13.3 and 3.24; 1.8, 2.13.4 and 3.24; 1.8, 2.13.3 and 3.25; 1.8, 2.13.4 and 3.25; 1.8, 2.13.3 and 3.26; 1.8, 2.13.4 and 3.26; 1.8, 2.13.3 and 3.27; 1.8, 2.13.4 and 3.27; 1.8, 2.13.3 and 3.28; 1.8, 2.13.4 and 3.28; 1.8, 2.13.3 and 3.29; 1.8, 2.13.4 and 3.29; 1.8, 2.13.3 and 3.30; 1.8, 2.13.4 and 3.30; 1.8, 2.13.3 and 3.31; 1.8, 2.13.4 and 3.31; 1.8, 2.13.3 and 3.33; 1.8, 2.13.4 and 3.33; 1.8, 2.13.3 and 3.34; 1.8, 2.13.4 and 3.34; 1.8, 2.13.3 and 3.35; 1.8, 2.13.4 and 3.35; 1.8, 2.13.3 and 3.36; 1.8, 2.13.4 and 3.36; 1.8, 2.13.3 and 3.37; 1.8, 2.13.3 and 3.38; 1.8, 2.13.4 and 3.37; 1.8, 2.13.4 and 3.38; 1.23, 2.1 and 3.1; 1.23, 2.2 and 3.1; 1.23, 2.3 and 3.1; 1.23, 2.1 and 3.3; 1.23, 2.2 and 3.3; 1.23, 2.3 and 3.3; 1.23, 2.1 and 3.4; 1.23, 2.2 and 3.4; 1.23, 2.3 and 3.4; 1.23, 2.1 and 3.8; 1.23, 2.2 and 3.8; 1.23, 2.3 and 3.8; 1.23, 2.1 and 3.9; 1.23, 2.2 and 3.9; 1.23, 2.3 and 3.9; 1.23, 2.1 and 3.16; 1.23, 2.2 and 3.16; 1.23, 2.3 and 3.16; 1.23, 2.1 and 3.17; 1.23, 2.2 and 3.17; 1.23, 2.3 and 3.17; 1.23, 2.1 and 3.20; 1.23, 2.2 and 3.20; 1.23, 2.3 and 3.20; 1.23, 2.1 and 3.24; 1.23, 2.2 and 3.24; 1.23, 2.3 and 3.24; 1.23, 2.1 and 3.25; 1.23, 2.2 and 3.25; 1.23, 2.3 and 3.25; 1.23, 2.1 and 3.26; 1.23, 2.2 and 3.26; 1.23, 2.3 and 3.26; 1.23, 2.1 and 3.27; 1.23, 2.2 and 3.27; 1.23, 2.3 and 3.27; 1.23, 2.1 and 3.28; 1.23, 2.2 and 3.28; 1.23, 2.3 and 3.28; 1.23, 2.1 and 3.29; 1.23, 2.2 and 3.29; 1.23, 2.3 and 3.29; 1.23, 2.1 and 3.30; 1.23, 2.2 and 3.30; 1.23, 2.3 and 3.30; 1.23, 2.1 and 3.31; 1.23, 2.2 and 3.31; 1.23, 2.3 and 3.31; 1.23, 2.1 and 3.33; 1.23, 2.2 and 3.33; 1.23, 2.3 and 3.33; 1.23, 2.1 and 3.34; 1.23, 2.2 and 3.34; 1.23, 2.3 and 3.34; 1.23, 2.1 and 3.35; 1.23, 2.2 and 3.35; 1.23, 2.3 and 3.35; 1.23, 2.1 and 3.36; 1.23, 2.2 and 3.36; 1.23, 2.3 and 3.36; 1.23, 2.1 and 3.37; 1.23, 2.1 and 3.38; 1.23, 2.2 and 3.37; 1.23, 2.2 and 3.38; 1.23, 2.3 and 3.37; 1.23, 2.3 and 3.38; 1.23, 2.4 and 3.1; 1.23, 2.5 and 3.1; 1.23, 2.7 and 3.1; 1.23, 2.4 and 3.3; 1.23, 2.5 and 3.3; 1.23, 2.7 and 3.3; 1.23, 2.4 and 3.4; 1.23, 2.5 and 3.4; 1.23, 2.7 and 3.4; 1.23, 2.4 and 3.8; 1.23, 2.5 and 3.8; 1.23, 2.7 and 3.8; 1.23, 2.4 and 3.9; 1.23, 2.5 and 3.9; 1.23, 2.7 and 3.9; 1.23, 2.4 and 3.16; 1.23, 2.5 and 3.16; 1.23, 2.7 and 3.16; 1.23, 2.4 and 3.17; 1.23, 2.5 and 3.17; 1.23, 2.7 and 3.17; 1.23, 2.4 and 3.20; 1.23, 2.5 and 3.20; 1.23, 2.7 and 3.20; 1.23, 2.4 and 3.24; 1.23, 2.5 and 3.24; 1.23, 2.7 and 3.24; 1.23, 2.4 and 3.25; 1.23, 2.5 and 3.25; 1.23, 2.7 and 3.25; 1.23, 2.4 and 3.26; 1.23, 2.5 and 3.26; 1.23, 2.7 and 3.26; 1.23, 2.4 and 3.27; 1.23, 2.5 and 3.27; 1.23, 2.7 and 3.27; 1.23, 2.4 and 3.28; 1.23, 2.5 and 3.28; 1.23, 2.7 and 3.28; 1.23, 2.4 and 3.29; 1.23, 2.5 and 3.29; 1.23, 2.7 and 3.29; 1.23, 2.4 and 3.30; 1.23, 2.5 and 3.30; 1.23, 2.7 and 3.30; 1.23, 2.4 and 3.31; 1.23, 2.5 and 3.31; 1.23, 2.7 and 3.31; 1.23, 2.4 and 3.33; 1.23, 2.5 and 3.33; 1.23, 2.7 and 3.33; 1.23, 2.4 and 3.34; 1.23, 2.5 and 3.34; 1.23, 2.7 and 3.34; 1.23, 2.4 and 3.35; 1.23, 2.5 and 3.35; 1.23, 2.7 and 3.35; 1.23, 2.4 and 3.36; 1.23, 2.5 and 3.36; 1.23, 2.7 and 3.36; 1.23, 2.4 and 3.37; 1.23, 2.4 and 3.38; 1.23, 2.5 and 3.37; 1.23, 2.5 and 3.38; 1.23, 2.7 and 3.37; 1.23, 2.7 and 3.38; 1.23, 2.9.1 and 3.1; 1.23, 2.9.2 and 3.1; 1.23, 2.9.1 and 3.3; 1.23, 2.9.2 and 3.3; 1.23, 2.9.1 and 3.4; 1.23, 2.9.2 and 3.4; 1.23, 2.9.1 and 3.8; 1.23, 2.9.2 and 3.8; 1.23, 2.9.1 and 3.9; 1.23, 2.9.2 and 3.9; 1.23, 2.9.1 and 3.16; 1.23, 2.9.2 and 3.16; 1.23, 2.9.1 and 3.17; 1.23, 2.9.2 and 3.17; 1.23, 2.9.1 and 3.20; 1.23, 2.9.2 and 3.20; 1.23, 2.9.1 and 3.24; 1.23, 2.9.2 and 3.24; 1.23, 2.9.1 and 3.25; 1.23, 2.9.2 and 3.25; 1.23, 2.9.1 and 3.26; 1.23, 2.9.2 and 3.26; 1.23, 2.9.1 and 3.27; 1.23, 2.9.2 and 3.27; 1.23, 2.9.1 and 3.28; 1.23, 2.9.2 and 3.28; 1.23, 2.9.1 and 3.29; 1.23, 2.9.2 and 3.29; 1.23, 2.9.1 and 3.30; 1.23, 2.9.2 and 3.30; 1.23, 2.9.1 and 3.31; 1.23, 2.9.2 and 3.31; 1.23, 2.9.1 and 3.33; 1.23, 2.9.2 and 3.33; 1.23, 2.9.1 and 3.34; 1.23, 2.9.2 and 3.34; 1.23, 2.9.1 and 3.35; 1.23, 2.9.2 and 3.35; 1.23, 2.9.1 and 3.36; 1.23, 2.9.2 and 3.36; 1.23, 2.9.1 and 3.37; 1.23, 2.9.1 and 3.38; 1.23, 2.9.2 and 3.37; 1.23, 2.9.2 and 3.38; 1.23, 2.10.1 and 3.1; 1.23, 2.10.2 and 3.1; 1.23, 2.10.1 and 3.3; 1.23, 2.10.2 and 3.3; 1.23, 2.10.1 and 3.4; 1.23, 2.10.2 and 3.4; 1.23, 2.10.1 and 3.8; 1.23, 2.10.2 and 3.8; 1.23, 2.10.1 and 3.9; 1.23, 2.10.2 and 3.9; 1.23, 2.10.1 and 3.16; 1.23, 2.10.2 and 3.16; 1.23, 2.10.1 and 3.17; 1.23, 2.10.2 and 3.17; 1.23, 2.10.1 and 3.20; 1.23, 2.10.2 and 3.20; 1.23, 2.10.1 and 3.24; 1.23, 2.10.2 and 3.24; 1.23, 2.10.1 and 3.25; 1.23, 2.10.2 and 3.25; 1.23, 2.10.1 and 3.26; 1.23, 2.10.2 and 3.26; 1.23, 2.10.1 and 3.27; 1.23, 2.10.2 and 3.27; 1.23, 2.10.1 and 3.28; 1.23, 2.10.2 and 3.28; 1.23, 2.10.1 and 3.29; 1.23, 2.10.2 and 3.29; 1.23, 2.10.1 and 3.30; 1.23, 2.10.2 and 3.30; 1.23, 2.10.1 and 3.31; 1.23, 2.10.2 and 3.31; 1.23, 2.10.1 and 3.33; 1.23, 2.10.2 and 3.33; 1.23, 2.10.1 and 3.34; 1.23, 2.10.2 and 3.34; 1.23, 2.10.1 and 3.35; 1.23, 2.10.2 and 3.35; 1.23, 2.10.1 and 3.36; 1.23, 2.10.2 and 3.36; 1.23, 2.10.1 and 3.37; 1.23, 2.10.1 and 3.38; 1.23, 2.10.2 and 3.37; 1.23, 2.10.2 and 3.38; 1.23, 2.11.5 and 3.1; 1.23, 2.11.5 and 3.3; 1.23, 2.11.5 and 3.4; 1.23, 2.11.5 and 3.8; 1.23, 2.11.5 and 3.9; 1.23, 2.11.5 and 3.16; 1.23, 2.11.5 and 3.17; 1.23, 2.11.5 and 3.20; 1.23, 2.11.5 and 3.24; 1.23, 2.11.5 and 3.25; 1.23, 2.11.5 and 3.26; 1.23, 2.11.5 and 3.27; 1.23, 2.11.5 and 3.28; 1.23, 2.11.5 and 3.29; 1.23, 2.11.5 and 3.30; 1.23, 2.11.5 and 3.31; 1.23, 2.11.5 and 3.33; 1.23, 2.11.5 and 3.34; 1.23, 2.11.5 and 3.35; 1.23, 2.11.5 and 3.36; 1.23, 2.11.5 and 3.37; 1.23, 2.11.5 and 3.38; 1.23, 2.11.6 and 3.1; 1.23, 2.12.1 and 3.1; 1.23, 2.12.2 and 3.1; 1.23, 2.11.6 and 3.3; 1.23, 2.12.1 and 3.3; 1.23, 2.12.2 and 3.3; 1.23, 2.11.6 and 3.4; 1.23, 2.12.1 and 3.4; 1.23, 2.12.2 and 3.4; 1.23, 2.11.6 and 3.8; 1.23, 2.12.1 and 3.8; 1.23, 2.12.2 and 3.8; 1.23, 2.11.6 and 3.9; 1.23, 2.12.1 and 3.9; 1.23, 2.12.2 and 3.9; 1.23, 2.11.6 and 3.16; 1.23, 2.12.1 and 3.16; 1.23, 2.12.2 and 3.16; 1.23, 2.11.6 and 3.17; 1.23, 2.12.1 and 3.17; 1.23, 2.12.2 and 3.17; 1.23, 2.11.6 and 3.20; 1.23, 2.12.1 and 3.20; 1.23, 2.12.2 and 3.20; 1.23, 2.11.6 and 3.24; 1.23, 2.12.1 and 3.24; 1.23, 2.12.2 and 3.24; 1.23, 2.11.6 and 3.25; 1.23, 2.12.1 and 3.25; 1.23, 2.12.2 and 3.25; 1.23, 2.11.6 and 3.26; 1.23, 2.12.1 and 3.26; 1.23, 2.12.2 and 3.26; 1.23, 2.11.6 and 3.27; 1.23, 2.12.1 and 3.27; 1.23, 2.12.2 and 3.27; 1.23, 2.11.6 and 3.28; 1.23, 2.12.1 and 3.28; 1.23, 2.12.2 and 3.28; 1.23, 2.11.6 and 3.29; 1.23, 2.12.1 and 3.29; 1.23, 2.12.2 and 3.29; 1.23, 2.11.6 and 3.30; 1.23, 2.12.1 and 3.30; 1.23, 2.12.2 and 3.30; 1.23, 2.11.6 and 3.31; 1.23, 2.12.1 and 3.31; 1.23, 2.12.2 and 3.31; 1.23, 2.11.6 and 3.33; 1.23, 2.12.1 and 3.33; 1.23, 2.12.2 and 3.33; 1.23, 2.11.6 and 3.34; 1.23, 2.12.1 and 3.34; 1.23, 2.12.2 and 3.34; 1.23, 2.11.6 and 3.35; 1.23, 2.12.1 and 3.35; 1.23, 2.12.2 and 3.35; 1.23, 2.11.6 and 3.36; 1.23, 2.12.1 and 3.36; 1.23, 2.12.2 and 3.36; 1.23, 2.11.6 and 3.37; 1.23, 2.11.6 and 3.38; 1.23, 2.12.1 and 3.37; 1.23, 2.12.1 and 3.38; 1.23, 2.12.2 and 3.37; 1.23, 2.12.2 and 3.38; 1.23, 2.13.3 and 3.1; 1.23, 2.13.4 and 3.1; 1.23, 2.13.3 and 3.3; 1.23, 2.13.4 and 3.3; 1.23, 2.13.3 and 3.4; 1.23, 2.13.4 and 3.4; 1.23, 2.13.3 and 3.8; 1.23, 2.13.4 and 3.8; 1.23, 2.13.3 and 3.9; 1.23, 2.13.4 and 3.9; 1.23, 2.13.3 and 3.16; 1.23, 2.13.4 and 3.16; 1.23, 2.13.3 and 3.17; 1.23, 2.13.4 and 3.17; 1.23, 2.13.3 and 3.20; 1.23, 2.13.4 and 3.20; 1.23, 2.13.3 and 3.24; 1.23, 2.13.4 and 3.24; 1.23, 2.13.3 and 3.25; 1.23, 2.13.4 and 3.25; 1.23, 2.13.3 and 3.26; 1.23, 2.13.4 and 3.26; 1.23, 2.13.3 and 3.27; 1.23, 2.13.4 and 3.27; 1.23, 2.13.3 and 3.28; 1.23, 2.13.4 and 3.28; 1.23, 2.13.3 and 3.29; 1.23, 2.13.4 and 3.29; 1.23, 2.13.3 and 3.30; 1.23, 2.13.4 and 3.30; 1.23, 2.13.3 and 3.31; 1.23, 2.13.4 and 3.31; 1.23, 2.13.3 and 3.33; 1.23, 2.13.4 and 3.33; 1.23, 2.13.3 and 3.34; 1.23, 2.13.4 and 3.34; 1.23, 2.13.3 and 3.35; 1.23, 2.13.4 and 3.35; 1.23, 2.13.3 and 3.36; 1.23, 2.13.4 and 3.36; 1.23, 2.13.3 and 3.37; 1.23, 2.13.3 and 3.38; 1.23, 2.13.4 and 3.37; 1.23, 2.13.4 and 3.38; 1.30, 2.1 and 3.1; 1.30, 2.2 and 3.1; 1.30, 2.3 and 3.1; 1.30, 2.1 and 3.3; 1.30, 2.2 and 3.3; 1.30, 2.3 and 3.3; 1.30, 2.1 and 3.4; 1.30, 2.2 and 3.4; 1.30, 2.3 and 3.4; 1.30, 2.1 and 3.8; 1.30, 2.2 and 3.8; 1.30, 2.3 and 3.8; 1.30, 2.1 and 3.9; 1.30, 2.2 and 3.9; 1.30, 2.3 and 3.9; 1.30, 2.1 and 3.16; 1.30, 2.2 and 3.16; 1.30, 2.3 and 3.16; 1.30, 2.1 and 3.17; 1.30, 2.2 and 3.17; 1.30, 2.3 and 3.17; 1.30, 2.1 and 3.20; 1.30, 2.2 and 3.20; 1.30, 2.3 and 3.20; 1.30, 2.1 and 3.24; 1.30, 2.2 and 3.24; 1.30, 2.3 and 3.24; 1.30, 2.1 and 3.25; 1.30, 2.2 and 3.25; 1.30, 2.3 and 3.25; 1.30, 2.1 and 3.26; 1.30, 2.2 and 3.26; 1.30, 2.3 and 3.26; 1.30, 2.1 and 3.27; 1.30, 2.2 and 3.27; 1.30, 2.3 and 3.27; 1.30, 2.1 and 3.28; 1.30, 2.2 and 3.28; 1.30, 2.3 and 3.28; 1.30, 2.1 and 3.29; 1.30, 2.2 and 3.29; 1.30, 2.3 and 3.29; 1.30, 2.1 and 3.30; 1.30, 2.2 and 3.30; 1.30, 2.3 and 3.30; 1.30, 2.1 and 3.31; 1.30, 2.2 and 3.31; 1.30, 2.3 and 3.31; 1.30, 2.1 and 3.33; 1.30, 2.2 and 3.33; 1.30, 2.3 and 3.33; 1.30, 2.1 and 3.34; 1.30, 2.2 and 3.34; 1.30, 2.3 and 3.34; 1.30, 2.1 and 3.35; 1.30, 2.2 and 3.35; 1.30, 2.3 and 3.35; 1.30, 2.1 and 3.36; 1.30, 2.2 and 3.36; 1.30, 2.3 and 3.36; 1.30, 2.1 and 3.37; 1.30, 2.1 and 3.38; 1.30, 2.2 and 3.37; 1.30, 2.2 and 3.38; 1.30, 2.3 and 3.37; 1.30, 2.3 and 3.38; 1.30, 2.4 and 3.1; 1.30, 2.5 and 3.1; 1.30, 2.7 and 3.1; 1.30, 2.4 and 3.3; 1.30, 2.5 and 3.3; 1.30, 2.7 and 3.3; 1.30, 2.4 and 3.4; 1.30, 2.5 and 3.4; 1.30, 2.7 and 3.4; 1.30, 2.4 and 3.8; 1.30, 2.5 and 3.8; 1.30, 2.7 and 3.8; 1.30, 2.4 and 3.9; 1.30, 2.5 and 3.9; 1.30, 2.7 and 3.9; 1.30, 2.4 and 3.16; 1.30, 2.5 and 3.16; 1.30, 2.7 and 3.16; 1.30, 2.4 and 3.17; 1.30, 2.5 and 3.17; 1.30, 2.7 and 3.17; 1.30, 2.4 and 3.20; 1.30, 2.5 and 3.20; 1.30, 2.7 and 3.20; 1.30, 2.4 and 3.24; 1.30, 2.5 and 3.24; 1.30, 2.7 and 3.24; 1.30, 2.4 and 3.25; 1.30, 2.5 and 3.25; 1.30, 2.7 and 3.25; 1.30, 2.4 and 3.26; 1.30, 2.5 and 3.26; 1.30, 2.7 and 3.26; 1.30, 2.4 and 3.27; 1.30, 2.5 and 3.27; 1.30, 2.7 and 3.27; 1.30, 2.4 and 3.28; 1.30, 2.5 and 3.28; 1.30, 2.7 and 3.28; 1.30, 2.4 and 3.29; 1.30, 2.5 and 3.29; 1.30, 2.7 and 3.29; 1.30, 2.4 and 3.30; 1.30, 2.5 and 3.30; 1.30, 2.7 and 3.30; 1.30, 2.4 and 3.31; 1.30, 2.5 and 3.31; 1.30, 2.7 and 3.31; 1.30, 2.4 and 3.33; 1.30, 2.5 and 3.33; 1.30, 2.7 and 3.33; 1.30, 2.4 and 3.34; 1.30, 2.5 and 3.34; 1.30, 2.7 and 3.34; 1.30, 2.4 and 3.35; 1.30, 2.5 and 3.35; 1.30, 2.7 and 3.35; 1.30, 2.4 and 3.36; 1.30, 2.5 and 3.36; 1.30, 2.7 and 3.36; 1.30, 2.4 and 3.37; 1.30, 2.4 and 3.38; 1.30, 2.5 and 3.37; 1.30, 2.5 and 3.38; 1.30, 2.7 and 3.37; 1.30, 2.7 and 3.38; 1.30, 2.9.1 and 3.1; 1.30, 2.9.2 and 3.1; 1.30, 2.9.1 and 3.3; 1.30, 2.9.2 and 3.3; 1.30, 2.9.1 and 3.4; 1.30, 2.9.2 and 3.4; 1.30, 2.9.1 and 3.8; 1.30, 2.9.2 and 3.8; 1.30, 2.9.1 and 3.9; 1.30, 2.9.2 and 3.9; 1.30, 2.9.1 and 3.16; 1.30, 2.9.2 and 3.16; 1.30, 2.9.1 and 3.17; 1.30, 2.9.2 and 3.17; 1.30, 2.9.1 and 3.20; 1.30, 2.9.2 and 3.20; 1.30, 2.9.1 and 3.24; 1.30, 2.9.2 and 3.24; 1.30, 2.9.1 and 3.25; 1.30, 2.9.2 and 3.25; 1.30, 2.9.1 and 3.26; 1.30, 2.9.2 and 3.26; 1.30, 2.9.1 and 3.27; 1.30, 2.9.2 and 3.27; 1.30, 2.9.1 and 3.28; 1.30, 2.9.2 and 3.28; 1.30, 2.9.1 and 3.29; 1.30, 2.9.2 and 3.29; 1.30, 2.9.1 and 3.30; 1.30, 2.9.2 and 3.30; 1.30, 2.9.1 and 3.31; 1.30, 2.9.2 and 3.31; 1.30, 2.9.1 and 3.33; 1.30, 2.9.2 and 3.33; 1.30, 2.9.1 and 3.34; 1.30, 2.9.2 and 3.34; 1.30, 2.9.1 and 3.35; 1.30, 2.9.2 and 3.35; 1.30, 2.9.1 and 3.36; 1.30, 2.9.2 and 3.36; 1.30, 2.9.1 and 3.37; 1.30, 2.9.1 and 3.38; 1.30, 2.9.2 and 3.37; 1.30, 2.9.2 and 3.38; 1.30, 2.10.1 and 3.1; 1.30, 2.10.2 and 3.1; 1.30, 2.10.1 and 3.3; 1.30, 2.10.2 and 3.3; 1.30, 2.10.1 and 3.4; 1.30, 2.10.2 and 3.4; 1.30, 2.10.1 and 3.8; 1.30, 2.10.2 and 3.8; 1.30, 2.10.1 and 3.9; 1.30, 2.10.2 and 3.9; 1.30, 2.10.1 and 3.16; 1.30, 2.10.2 and 3.16; 1.30, 2.10.1 and 3.17; 1.30, 2.10.2 and 3.17; 1.30, 2.10.1 and 3.20; 1.30, 2.10.2 and 3.20; 1.30, 2.10.1 and 3.24; 1.30, 2.10.2 and 3.24; 1.30, 2.10.1 and 3.25; 1.30, 2.10.2 and 3.25; 1.30, 2.10.1 and 3.26; 1.30, 2.10.2 and 3.26; 1.30, 2.10.1 and 3.27; 1.30, 2.10.2 and 3.27; 1.30, 2.10.1 and 3.28; 1.30, 2.10.2 and 3.28; 1.30, 2.10.1 and 3.29; 1.30, 2.10.2 and 3.29; 1.30, 2.10.1 and 3.30; 1.30, 2.10.2 and 3.30; 1.30, 2.10.1 and 3.31; 1.30, 2.10.2 and 3.31; 1.30, 2.10.1 and 3.33; 1.30, 2.10.2 and 3.33; 1.30, 2.10.1 and 3.34; 1.30, 2.10.2 and 3.34; 1.30, 2.10.1 and 3.35; 1.30, 2.10.2 and 3.35; 1.30, 2.10.1 and 3.36; 1.30, 2.10.2 and 3.36; 1.30, 2.10.1 and 3.37; 1.30, 2.10.1 and 3.38; 1.30, 2.10.2 and 3.37; 1.30, 2.10.2 and 3.38; 1.30, 2.11.5 and 3.1; 1.30, 2.11.5 and 3.3; 1.30, 2.11.5 and 3.4; 1.30, 2.11.5 and 3.8; 1.30, 2.11.5 and 3.9; 1.30, 2.11.5 and 3.16; 1.30, 2.11.5 and 3.17; 1.30, 2.11.5 and 3.20; 1.30, 2.11.5 and 3.24; 1.30, 2.11.5 and 3.25, 1.30, 2.11.5 and 3.26; 1.30, 2.11.5 and 3.27; 1.30, 2.11.5 and 3.28; 1.30, 2.11.5 and 3.29; 1.30, 2.11.5 and 3.30; 1.30, 2.11.5 and 3.31; 1.30, 2.11.5 and 3.33; 1.30, 2.11.5 and 3.34; 1.30, 2.11.5 and 3.35; 1.30, 2.11.5 and 3.36; 1.30, 2.11.5 and 3.37; 1.30, 2.11.5 and 3.38; 1.30, 2.11.6 and 3.1; 1.30, 2.12.1 and 3.1; 1.30, 2.12.2 and 3.1; 1.30, 2.11.6 and 3.3; 1.30, 2.12.1 and 3.3; 1.30, 2.12.2 and 3.3; 1.30, 2.11.6 and 3.4; 1.30, 2.12.1 and 3.4; 1.30, 2.12.2 and 3.4; 1.30, 2.11.6 and 3.8; 1.30, 2.12.1 and 3.8; 1.30, 2.12.2 and 3.8, 1.30, 2.11.6 and 3.9; 1.30, 2.12.1 and 3.9; 1.30, 2.12.2 and 3.9; 1.30, 2.11.6 and 3.16, 1.30, 2.12.1 and 3.16; 1.30, 2.12.2 and 3.16; 1.30, 2.11.6 and 3.17; 1.30, 2.12.1 and 3.17, 1.30, 2.12.2 and 3.17; 1.30, 2.11.6 and 3.20; 1.30, 2.12.1 and 3.20; 1.30, 2.12.2 and 3.20, 1.30, 2.11.6 and 3.24; 1.30, 2.12.1 and 3.24; 1.30, 2.12.2 and 3.24; 1.30, 2.11.6 and 3.25, 1.30, 2.12.1 and 3.25; 1.30, 2.12.2 and 3.25; 1.30, 2.11.6 and 3.26; 1.30, 2.12.1 and 3.26, 1.30, 2.12.2 and 3.26; 1.30, 2.11.6 and 3.27; 1.30, 2.12.1 and 3.27; 1.30, 2.12.2 and 3.27; 1.30, 2.11.6 and 3.28; 1.30, 2.12.1 and 3.28; 1.30, 2.12.2 and 3.28; 1.30, 2.11.6 and 3.29, 1.30, 2.12.1 and 3.29; 1.30, 2.12.2 and 3.29; 1.30, 2.11.6 and 3.30; 1.30, 2.12.1 and 3.30, 1.30, 2.12.2 and 3.30; 1.30, 2.11.6 and 3.31; 1.30, 2.12.1 and 3.31; 1.30, 2.12.2 and 3.31, 1.30, 2.11.6 and 3.33; 1.30, 2.12.1 and 3.33; 1.30, 2.12.2 and 3.33; 1.30, 2.11.6 and 3.34, 1.30, 2.12.1 and 3.34; 1.30, 2.12.2 and 3.34; 1.30, 2.11.6 and 3.35; 1.30, 2.12.1 and 3.35, 1.30, 2.12.2 and 3.35; 1.30, 2.11.6 and 3.36; 1.30, 2.12.1 and 3.36; 1.30, 2.12.2 and 3.36; 1.30, 2.11.6 and 3.37; 1.30, 2.11.6 and 3.38; 1.30, 2.12.1 and 3.37; 1.30, 2.12.1 and 3.38; 1.30, 2.12.2 and 3.37; 1.30, 2.12.2 and 3.38; 1.30, 2.13.3 and 3.1; 1.30, 2.13.4 and 3.1; 1.30, 2.13.3 and 3.3; 1.30, 2.13.4 and 3.3; 1.30, 2.13.3 and 3.4; 1.30, 2.13.4 and 3.4; 1.30, 2.13.3 and 3.8; 1.30, 2.13.4 and 3.8; 1.30, 2.13.3 and 3.9; 1.30, 2.13.4 and 3.9; 1.30, 2.13.3 and 3.16; 1.30, 2.13.4 and 3.16; 1.30, 2.13.3 and 3.17; 1.30, 2.13.4 and 3.17; 1.30, 2.13.3 and 3.20; 1.30, 2.13.4 and 3.20; 1.30, 2.13.3 and 3.24; 1.30, 2.13.4 and 3.24; 1.30, 2.13.3 and 3.25; 1.30, 2.13.4 and 3.25; 1.30, 2.13.3 and 3.26; 1.30, 2.13.4 and 3.26; 1.30, 2.13.3 and 3.27; 1.30, 2.13.4 and 3.27; 1.30, 2.13.3 and 3.28; 1.30, 2.13.4 and 3.28; 1.30, 2.13.3 and 3.29; 1.30, 2.13.4 and 3.29; 1.30, 2.13.3 and 3.30; 1.30, 2.13.4 and 3.30; 1.30, 2.13.3 and 3.31; 1.30, 2.13.4 and 3.31; 1.30, 2.13.3 and 3.33; 1.30, 2.13.4 and 3.33; 1.30, 2.13.3 and 3.34; 1.30, 2.13.4 and 3.34; 1.30, 2.13.3 and 3.35; 1.30, 2.13.4 and 3.35; 1.30, 2.13.3 and 3.36; 1.30, 2.13.4 and 3.36; 1.30, 2.13.3 and 3.37; 1.30, 2.13.3 and 3.38; 1.30, 2.13.4 and 3.37; 1.30, 2.13.4 and 3.38; 1.33, 2.1 and 3.1; 1.33, 2.2 and 3.1; 1.33, 2.3 and 3.1; 1.33, 2.1 and 3.3; 1.33, 2.2 and 3.3; 1.33, 2.3 and 3.3; 1.33, 2.1 and 3.4; 1.33, 2.2 and 3.4; 1.33, 2.3 and 3.4; 1.33, 2.1 and 3.8; 1.33, 2.2 and 3.8; 1.33, 2.3 and 3.8; 1.33, 2.1 and 3.9; 1.33, 2.2 and 3.9; 1.33, 2.3 and 3.9; 1.33, 2.1 and 3.16; 1.33, 2.2 and 3.16; 1.33, 2.3 and 3.16; 1.33, 2.1 and 3.17; 1.33, 2.2 and 3.17; 1.33, 2.3 and 3.17; 1.33, 2.1 and 3.20; 1.33, 2.2 and 3.20; 1.33, 2.3 and 3.20; 1.33, 2.1 and 3.24; 1.33, 2.2 and 3.24; 1.33, 2.3 and 3.24; 1.33, 2.1 and 3.25; 1.33, 2.2 and 3.25; 1.33, 2.3 and 3.25; 1.33, 2.1 and 3.26; 1.33, 2.2 and 3.26; 1.33, 2.3 and 3.26; 1.33, 2.1 and 3.27; 1.33, 2.2 and 3.27; 1.33, 2.3 and 3.27; 1.33, 2.1 and 3.28; 1.33, 2.2 and 3.28; 1.33, 2.3 and 3.28; 1.33, 2.1 and 3.29; 1.33, 2.2 and 3.29; 1.33, 2.3 and 3.29; 1.33, 2.1 and 3.30; 1.33, 2.2 and 3.30; 1.33, 2.3 and 3.30; 1.33, 2.1 and 3.31; 1.33, 2.2 and 3.31; 1.33, 2.3 and 3.31; 1.33, 2.1 and 3.33; 1.33, 2.2 and 3.33; 1.33, 2.3 and 3.33; 1.33, 2.1 and 3.34; 1.33, 2.2 and 3.34; 1.33, 2.3 and 3.34; 1.33, 2.1 and 3.35; 1.33, 2.2 and 3.35; 1.33, 2.3 and 3.35; 1.33, 2.1 and 3.36; 1.33, 2.2 and 3.36; 1.33, 2.3 and 3.36; 1.33, 2.1 and 3.37; 1.33, 2.1 and 3.38; 1.33, 2.2 and 3.37; 1.33, 2.2 and 3.38; 1.33, 2.3 and 3.37; 1.33, 2.3 and 3.38; 1.33, 2.4 and 3.1; 1.33, 2.5 and 3.1; 1.33, 2.7 and 3.1; 1.33, 2.4 and 3.3; 1.33, 2.5 and 3.3; 1.33, 2.7 and 3.3; 1.33, 2.4 and 3.4; 1.33, 2.5 and 3.4; 1.33, 2.7 and 3.4; 1.33, 2.4 and 3.8; 1.33, 2.5 and 3.8; 1.33, 2.7 and 3.8; 1.33, 2.4 and 3.9; 1.33, 2.5 and 3.9; 1.33, 2.7 and 3.9; 1.33, 2.4 and 3.16; 1.33, 2.5 and 3.16; 1.33, 2.7 and 3.16; 1.33, 2.4 and 3.17; 1.33, 2.5 and 3.17; 1.33, 2.7 and 3.17; 1.33, 2.4 and 3.20; 1.33, 2.5 and 3.20; 1.33, 2.7 and 3.20; 1.33, 2.4 and 3.24; 1.33, 2.5 and 3.24; 1.33, 2.7 and 3.24; 1.33, 2.4 and 3.25; 1.33, 2.5 and 3.25; 1.33, 2.7 and 3.25; 1.33, 2.4 and 3.26; 1.33, 2.5 and 3.26; 1.33, 2.7 and 3.26; 1.33, 2.4 and 3.27; 1.33, 2.5 and 3.27; 1.33, 2.7 and 3.27; 1.33, 2.4 and 3.28; 1.33, 2.5 and 3.28; 1.33, 2.7 and 3.28; 1.33, 2.4 and 3.29; 1.33, 2.5 and 3.29; 1.33, 2.7 and 3.29; 1.33, 2.4 and 3.30; 1.33, 2.5 and 3.30; 1.33, 2.7 and 3.30; 1.33, 2.4 and 3.31; 1.33, 2.5 and 3.31; 1.33, 2.7 and 3.31; 1.33, 2.4 and 3.33; 1.33, 2.5 and 3.33; 1.33, 2.7 and 3.33; 1.33, 2.4 and 3.34; 1.33, 2.5 and 3.34; 1.33, 2.7 and 3.34; 1.33, 2.4 and 3.35; 1.33, 2.5 and 3.35; 1.33, 2.7 and 3.35; 1.33, 2.4 and 3.36; 1.33, 2.5 and 3.36; 1.33, 2.7 and 3.36; 1.33, 2.4 and 3.37; 1.33, 2.4 and 3.38; 1.33, 2.5 and 3.37; 1.33, 2.5 and 3.38; 1.33, 2.7 and 3.37; 1.33, 2.7 and 3.38; 1.33, 2.9.1 and 3.1; 1.33, 2.9.2 and 3.1; 1.33, 2.9.1 and 3.3; 1.33, 2.9.2 and 3.3; 1.33, 2.9.1 and 3.4; 1.33, 2.9.2 and 3.4; 1.33, 2.9.1 and 3.8; 1.33, 2.9.2 and 3.8; 1.33, 2.9.1 and 3.9; 1.33, 2.9.2 and 3.9; 1.33, 2.9.1 and 3.16; 1.33, 2.9.2 and 3.16; 1.33, 2.9.1 and 3.17; 1.33, 2.9.2 and 3.17; 1.33, 2.9.1 and 3.20; 1.33, 2.9.2 and 3.20; 1.33, 2.9.1 and 3.24; 1.33, 2.9.2 and 3.24; 1.33, 2.9.1 and 3.25; 1.33, 2.9.2 and 3.25; 1.33, 2.9.1 and 3.26; 1.33, 2.9.2 and 3.26; 1.33, 2.9.1 and 3.27; 1.33, 2.9.2 and 3.27; 1.33, 2.9.1 and 3.28; 1.33, 2.9.2 and 3.28; 1.33, 2.9.1 and 3.29; 1.33, 2.9.2 and 3.29; 1.33, 2.9.1 and 3.30; 1.33, 2.9.2 and 3.30; 1.33, 2.9.1 and 3.31; 1.33, 2.9.2 and 3.31; 1.33, 2.9.1 and 3.33; 1.33, 2.9.2 and 3.33; 1.33, 2.9.1 and 3.34; 1.33, 2.9.2 and 3.34; 1.33, 2.9.1 and 3.35; 1.33, 2.9.2 and 3.35; 1.33, 2.9.1 and 3.36; 1.33, 2.9.2 and 3.36; 1.33, 2.9.1 and 3.37; 1.33, 2.9.1 and 3.38; 1.33, 2.9.2 and 3.37; 1.33, 2.9.2 and 3.38; 1.33, 2.10.1 and 3.1; 1.33, 2.10.2 and 3.1; 1.33, 2.10.1 and 3.3; 1.33, 2.10.2 and 3.3; 1.33, 2.10.1 and 3.4; 1.33, 2.10.2 and 3.4; 1.33, 2.10.1 and 3.8; 1.33, 2.10.2 and 3.8; 1.33, 2.10.1 and 3.9; 1.33, 2.10.2 and 3.9; 1.33, 2.10.1 and 3.16; 1.33, 2.10.2 and 3.16; 1.33, 2.10.1 and 3.17; 1.33, 2.10.2 and 3.17; 1.33, 2.10.1 and 3.20; 1.33, 2.10.2 and 3.20; 1.33, 2.10.1 and 3.24; 1.33, 2.10.2 and 3.24; 1.33, 2.10.1 and 3.25; 1.33, 2.10.2 and 3.25; 1.33, 2.10.1 and 3.26; 1.33, 2.10.2 and 3.26; 1.33, 2.10.1 and 3.27; 1.33, 2.10.2 and 3.27; 1.33, 2.10.1 and 3.28; 1.33, 2.10.2 and 3.28; 1.33, 2.10.1 and 3.29; 1.33, 2.10.2 and 3.29; 1.33, 2.10.1 and 3.30; 1.33, 2.10.2 and 3.30; 1.33, 2.10.1 and 3.31; 1.33, 2.10.2 and 3.31; 1.33, 2.10.1 and 3.33; 1.33, 2.10.2 and 3.33; 1.33, 2.10.1 and 3.34; 1.33, 2.10.2 and 3.34; 1.33, 2.10.1 and 3.35; 1.33, 2.10.2 and 3.35; 1.33, 2.10.1 and 3.36; 1.33, 2.10.2 and 3.36; 1.33, 2.10.1 and 3.37; 1.33, 2.10.1 and 3.38; 1.33, 2.10.2 and 3.37; 1.33, 2.10.2 and 3.38; 1.33, 2.11.5 and 3.1; 1.33, 2.11.5 and 3.3; 1.33, 2.11.5 and 3.4; 1.33, 2.11.5 and 3.8; 1.33, 2.11.5 and 3.9; 1.33, 2.11.5 and 3.16; 1.33, 2.11.5 and 3.17; 1.33, 2.11.5 and 3.20; 1.33, 2.11.5 and 3.24; 1.33, 2.11.5 and 3.25; 1.33, 2.11.5 and 3.26; 1.33, 2.11.5 and 3.27; 1.33, 2.11.5 and 3.28; 1.33, 2.11.5 and 3.29; 1.33, 2.11.5 and 3.30; 1.33, 2.11.5 and 3.31; 1.33, 2.11.5 and 3.33; 1.33, 2.11.5 and 3.34; 1.33, 2.11.5 and 3.35; 1.33, 2.11.5 and 3.36; 1.33, 2.11.5 and 3.37; 1.33, 2.11.5 and 3.38; 1.33, 2.11.6 and 3.1; 1.33, 2.12.1 and 3.1; 1.33, 2.12.2 and 3.1; 1.33, 2.11.6 and 3.3; 1.33, 2.12.1 and 3.3; 1.33, 2.12.2 and 3.3; 1.33, 2.11.6 and 3.4; 1.33, 2.12.1 and 3.4; 1.33, 2.12.2 and 3.4; 1.33, 2.11.6 and 3.8; 1.33, 2.12.1 and 3.8; 1.33, 2.12.2 and 3.8; 1.33, 2.11.6 and 3.9; 1.33, 2.12.1 and 3.9; 1.33, 2.12.2 and 3.9; 1.33, 2.11.6 and 3.16; 1.33, 2.12.1 and 3.16; 1.33, 2.12.2 and 3.16; 1.33, 2.11.6 and 3.17; 1.33, 2.12.1 and 3.17; 1.33, 2.12.2 and 3.17; 1.33, 2.11.6 and 3.20; 1.33, 2.12.1 and 3.20; 1.33, 2.12.2 and 3.20; 1.33, 2.11.6 and 3.24; 1.33, 2.12.1 and 3.24; 1.33, 2.12.2 and 3.24; 1.33, 2.11.6 and 3.25; 1.33, 2.12.1 and 3.25; 1.33, 2.12.2 and 3.25; 1.33, 2.11.6 and 3.26; 1.33, 2.12.1 and 3.26; 1.33, 2.12.2 and 3.26; 1.33, 2.11.6 and 3.27; 1.33, 2.12.1 and 3.27; 1.33, 2.12.2 and 3.27; 1.33, 2.11.6 and 3.28; 1.33, 2.12.1 and 3.28; 1.33, 2.12.2 and 3.28; 1.33, 2.11.6 and 3.29; 1.33, 2.12.1 and 3.29; 1.33, 2.12.2 and 3.29; 1.33, 2.11.6 and 3.30; 1.33, 2.12.1 and 3.30; 1.33, 2.12.2 and 3.30; 1.33, 2.11.6 and 3.31; 1.33, 2.12.1 and 3.31; 1.33, 2.12.2 and 3.31; 1.33, 2.11.6 and 3.33; 1.33, 2.12.1 and 3.33; 1.33, 2.12.2 and 3.33; 1.33, 2.11.6 and 3.34; 1.33, 2.12.1 and 3.34; 1.33, 2.12.2 and 3.34; 1.33, 2.11.6 and 3.35; 1.33, 2.12.1 and 3.35; 1.33, 2.12.2 and 3.35; 1.33, 2.11.6 and 3.36; 1.33, 2.12.1 and 3.36; 1.33, 2.12.2 and 3.36; 1.33, 2.11.6 and 3.37; 1.33, 2.11.6 and 3.38; 1.33, 2.12.1 and 3.37; 1.33, 2.12.1 and 3.38; 1.33, 2.12.2 and 3.37; 1.33, 2.12.2 and 3.38; 1.33, 2.13.3 and 3.1; 1.33, 2.13.4 and 3.1; 1.33, 2.13.3 and 3.3; 1.33, 2.13.4 and 3.3; 1.33, 2.13.3 and 3.4; 1.33, 2.13.4 and 3.4; 1.33, 2.13.3 and 3.8; 1.33, 2.13.4 and 3.8; 1.33, 2.13.3 and 3.9; 1.33, 2.13.4 and 3.9; 1.33, 2.13.3 and 3.16; 1.33, 2.13.4 and 3.16; 1.33, 2.13.3 and 3.17; 1.33, 2.13.4 and 3.17; 1.33, 2.13.3 and 3.20; 1.33, 2.13.4 and 3.20; 1.33, 2.13.3 and 3.24; 1.33, 2.13.4 and 3.24; 1.33, 2.13.3 and 3.25; 1.33, 2.13.4 and 3.25; 1.33, 2.13.3 and 3.26; 1.33, 2.13.4 and 3.26; 1.33, 2.13.3 and 3.27; 1.33, 2.13.4 and 3.27; 1.33, 2.13.3 and 3.28; 1.33, 2.13.4 and 3.28; 1.33, 2.13.3 and 3.29; 1.33, 2.13.4 and 3.29; 1.33, 2.13.3 and 3.30; 1.33, 2.13.4 and 3.30; 1.33, 2.13.3 and 3.31; 1.33, 2.13.4 and 3.31; 1.33, 2.13.3 and 3.33; 1.33, 2.13.4 and 3.33; 1.33, 2.13.3 and 3.34; 1.33, 2.13.4 and 3.34; 1.33, 2.13.3 and 3.35; 1.33, 2.13.4 and 3.35; 1.33, 2.13.3 and 3.36; 1.33, 2.13.4 and 3.36; 1.33, 2.13.3 and 3.37; 1.33, 2.13.3 and 3.38; 1.33, 2.13.4 and 3.37; 1.33, 2.13.4 and 3.38; 1.34, 2.1 and 3.1; 1.34, 2.2 and 3.1; 1.34, 2.3 and 3.1; 1.34, 2.1 and 3.3; 1.34, 2.2 and 3.3; 1.34, 2.3 and 3.3; 1.34, 2.1 and 3.4; 1.34, 2.2 and 3.4; 1.34, 2.3 and 3.4; 1.34, 2.1 and 3.8; 1.34, 2.2 and 3.8; 1.34, 2.3 and 3.8; 1.34, 2.1 and 3.9; 1.34, 2.2 and 3.9; 1.34, 2.3 and 3.9; 1.34, 2.1 and 3.16; 1.34, 2.2 and 3.16; 1.34, 2.3 and 3.16; 1.34, 2.1 and 3.17; 1.34, 2.2 and 3.17; 1.34, 2.3 and 3.17; 1.34, 2.1 and 3.20; 1.34, 2.2 and 3.20; 1.34, 2.3 and 3.20; 1.34, 2.1 and 3.24; 1.34, 2.2 and 3.24; 1.34, 2.3 and 3.24; 1.34, 2.1 and 3.25; 1.34, 2.2 and 3.25; 1.34, 2.3 and 3.25; 1.34, 2.1 and 3.26; 1.34, 2.2 and 3.26; 1.34, 2.3 and 3.26; 1.34, 2.1 and 3.27; 1.34, 2.2 and 3.27; 1.34, 2.3 and 3.27; 1.34, 2.1 and 3.28; 1.34, 2.2 and 3.28; 1.34, 2.3 and 3.28; 1.34, 2.1 and 3.29; 1.34, 2.2 and 3.29; 1.34, 2.3 and 3.29; 1.34, 2.1 and 3.30; 1.34, 2.2 and 3.30; 1.34, 2.3 and 3.30; 1.34, 2.1 and 3.31; 1.34, 2.2 and 3.31; 1.34, 2.3 and 3.31; 1.34, 2.1 and 3.33; 1.34, 2.2 and 3.33; 1.34, 2.3 and 3.33; 1.34, 2.1 and 3.34; 1.34, 2.2 and 3.34; 1.34, 2.3 and 3.34; 1.34, 2.1 and 3.35; 1.34, 2.2 and 3.35; 1.34, 2.3 and 3.35; 1.34, 2.1 and 3.36; 1.34, 2.2 and 3.36; 1.34, 2.3 and 3.36; 1.34, 2.1 and 3.37; 1.34, 2.1 and 3.38; 1.34, 2.2 and 3.37; 1.34, 2.2 and 3.38; 1.34, 2.3 and 3.37; 1.34, 2.3 and 3.38; 1.34, 2.4 and 3.1; 1.34, 2.5 and 3.1; 1.34, 2.7 and 3.1; 1.34, 2.4 and 3.3; 1.34, 2.5 and 3.3; 1.34, 2.7 and 3.3; 1.34, 2.4 and 3.4; 1.34, 2.5 and 3.4; 1.34, 2.7 and 3.4; 1.34, 2.4 and 3.8; 1.34, 2.5 and 3.8; 1.34, 2.7 and 3.8; 1.34, 2.4 and 3.9; 1.34, 2.5 and 3.9; 1.34, 2.7 and 3.9; 1.34, 2.4 and 3.16; 1.34, 2.5 and 3.16; 1.34, 2.7 and 3.16; 1.34, 2.4 and 3.17; 1.34, 2.5 and 3.17; 1.34, 2.7 and 3.17; 1.34, 2.4 and 3.20; 1.34, 2.5 and 3.20; 1.34, 2.7 and 3.20; 1.34, 2.4 and 3.24; 1.34, 2.5 and 3.24; 1.34, 2.7 and 3.24; 1.34, 2.4 and 3.25; 1.34, 2.5 and 3.25; 1.34, 2.7 and 3.25; 1.34, 2.4 and 3.26; 1.34, 2.5 and 3.26; 1.34, 2.7 and 3.26; 1.34, 2.4 and 3.27; 1.34, 2.5 and 3.27; 1.34, 2.7 and 3.27; 1.34, 2.4 and 3.28; 1.34, 2.5 and 3.28; 1.34, 2.7 and 3.28; 1.34, 2.4 and 3.29; 1.34, 2.5 and 3.29; 1.34, 2.7 and 3.29; 1.34, 2.4 and 3.30; 1.34, 2.5 and 3.30; 1.34, 2.7 and 3.30; 1.34, 2.4 and 3.31; 1.34, 2.5 and 3.31; 1.34, 2.7 and 3.31; 1.34, 2.4 and 3.33; 1.34, 2.5 and 3.33; 1.34, 2.7 and 3.33; 1.34, 2.4 and 3.34; 1.34, 2.5 and 3.34; 1.34, 2.7 and 3.34; 1.34, 2.4 and 3.35; 1.34, 2.5 and 3.35; 1.34, 2.7 and 3.35; 1.34, 2.4 and 3.36; 1.34, 2.5 and 3.36; 1.34, 2.7 and 3.36; 1.34, 2.4 and 3.37; 1.34, 2.4 and 3.38; 1.34, 2.5 and 3.37; 1.34, 2.5 and 3.38; 1.34, 2.7 and 3.37; 1.34, 2.7 and 3.38; 1.34, 2.9.1 and 3.1; 1.34, 2.9.2 and 3.1; 1.34, 2.9.1 and 3.3; 1.34, 2.9.2 and 3.3; 1.34, 2.9.1 and 3.4; 1.34, 2.9.2 and 3.4; 1.34, 2.9.1 and 3.8; 1.34, 2.9.2 and 3.8; 1.34, 2.9.1 and 3.9; 1.34, 2.9.2 and 3.9; 1.34, 2.9.1 and 3.16; 1.34, 2.9.2 and 3.16; 1.34, 2.9.1 and 3.17; 1.34, 2.9.2 and 3.17; 1.34, 2.9.1 and 3.20; 1.34, 2.9.2 and 3.20; 1.34, 2.9.1 and 3.24; 1.34, 2.9.2 and 3.24; 1.34, 2.9.1 and 3.25; 1.34, 2.9.2 and 3.25; 1.34, 2.9.1 and 3.26; 1.34, 2.9.2 and 3.26; 1.34, 2.9.1 and 3.27; 1.34, 2.9.2 and 3.27; 1.34, 2.9.1 and 3.28; 1.34, 2.9.2 and 3.28; 1.34, 2.9.1 and 3.29; 1.34, 2.9.2 and 3.29; 1.34, 2.9.1 and 3.30; 1.34, 2.9.2 and 3.30; 1.34, 2.9.1 and 3.31; 1.34, 2.9.2 and 3.31; 1.34, 2.9.1 and 3.33; 1.34, 2.9.2 and 3.33; 1.34, 2.9.1 and 3.34; 1.34, 2.9.2 and 3.34; 1.34, 2.9.1 and 3.35; 1.34, 2.9.2 and 3.35; 1.34, 2.9.1 and 3.36; 1.34, 2.9.2 and 3.36; 1.34, 2.9.1 and 3.37; 1.34, 2.9.1 and 3.38; 1.34, 2.9.2 and 3.37; 1.34, 2.9.2 and 3.38; 1.34, 2.10.1 and 3.1; 1.34, 2.10.2 and 3.1; 1.34, 2.10.1 and 3.3; 1.34, 2.10.2 and 3.3; 1.34, 2.10.1 and 3.4; 1.34, 2.10.2 and 3.4; 1.34, 2.10.1 and 3.8; 1.34, 2.10.2 and 3.8; 1.34, 2.10.1 and 3.9; 1.34, 2.10.2 and 3.9; 1.34, 2.10.1 and 3.16; 1.34, 2.10.2 and 3.16; 1.34, 2.10.1 and 3.17; 1.34, 2.10.2 and 3.17; 1.34, 2.10.1 and 3.20; 1.34, 2.10.2 and 3.20; 1.34, 2.10.1 and 3.24; 1.34, 2.10.2 and 3.24; 1.34, 2.10.1 and 3.25; 1.34, 2.10.2 and 3.25; 1.34, 2.10.1 and 3.26; 1.34, 2.10.2 and 3.26; 1.34, 2.10.1 and 3.27; 1.34, 2.10.2 and 3.27; 1.34, 2.10.1 and 3.28; 1.34, 2.10.2 and 3.28; 1.34, 2.10.1 and 3.29; 1.34, 2.10.2 and 3.29; 1.34, 2.10.1 and 3.30; 1.34, 2.10.2 and 3.30; 1.34, 2.10.1 and 3.31; 1.34, 2.10.2 and 3.31; 1.34, 2.10.1 and 3.33; 1.34, 2.10.2 and 3.33; 1.34, 2.10.1 and 3.34; 1.34, 2.10.2 and 3.34; 1.34, 2.10.1 and 3.35; 1.34, 2.10.2 and 3.35; 1.34, 2.10.1 and 3.36; 1.34, 2.10.2 and 3.36; 1.34, 2.10.1 and 3.37; 1.34, 2.10.1 and 3.38; 1.34, 2.10.2 and 3.37; 1.34, 2.10.2 and 3.38; 1.34, 2.11.5 and 3.1; 1.34, 2.11.5 and 3.3; 1.34, 2.11.5 and 3.4; 1.34, 2.11.5 and 3.8; 1.34, 2.11.5 and 3.9; 1.34, 2.11.5 and 3.16; 1.34, 2.11.5 and 3.17; 1.34, 2.11.5 and 3.20; 1.34, 2.11.5 and 3.24; 1.34, 2.11.5 and 3.25; 1.34, 2.11.5 and 3.26; 1.34, 2.11.5 and 3.27; 1.34, 2.11.5 and 3.28; 1.34, 2.11.5 and 3.29; 1.34, 2.11.5 and 3.30; 1.34, 2.11.5 and 3.31; 1.34, 2.11.5 and 3.33; 1.34, 2.11.5 and 3.34; 1.34, 2.11.5 and 3.35; 1.34, 2.11.5 and 3.36; 1.34, 2.11.5 and 3.37; 1.34, 2.11.5 and 3.38; 1.34, 2.11.6 and 3.1; 1.34, 2.12.1 and 3.1; 1.34, 2.12.2 and 3.1; 1.34, 2.11.6 and 3.3; 1.34, 2.12.1 and 3.3; 1.34, 2.12.2 and 3.3; 1.34, 2.11.6 and 3.4; 1.34, 2.12.1 and 3.4; 1.34, 2.12.2 and 3.4; 1.34, 2.11.6 and 3.8; 1.34, 2.12.1 and 3.8; 1.34, 2.12.2 and 3.8; 1.34, 2.11.6 and 3.9; 1.34, 2.12.1 and 3.9; 1.34, 2.12.2 and 3.9; 1.34, 2.11.6 and 3.16; 1.34, 2.12.1 and 3.16; 1.34, 2.12.2 and 3.16; 1.34, 2.11.6 and 3.17; 1.34, 2.12.1 and 3.17; 1.34, 2.12.2 and 3.17; 1.34, 2.11.6 and 3.20; 1.34, 2.12.1 and 3.20; 1.34, 2.12.2 and 3.20; 1.34, 2.11.6 and 3.24; 1.34, 2.12.1 and 3.24; 1.34, 2.12.2 and 3.24; 1.34, 2.11.6 and 3.25; 1.34, 2.12.1 and 3.25; 1.34, 2.12.2 and 3.25; 1.34, 2.11.6 and 3.26; 1.34, 2.12.1 and 3.26; 1.34, 2.12.2 and 3.26; 1.34, 2.11.6 and 3.27; 1.34, 2.12.1 and 3.27; 1.34, 2.12.2 and 3.27; 1.34, 2.11.6 and 3.28; 1.34, 2.12.1 and 3.28; 1.34, 2.12.2 and 3.28; 1.34, 2.11.6 and 3.29; 1.34, 2.12.1 and 3.29; 1.34, 2.12.2 and 3.29; 1.34, 2.11.6 and 3.30; 1.34, 2.12.1 and 3.30; 1.34, 2.12.2 and 3.30; 1.34, 2.11.6 and 3.31; 1.34, 2.12.1 and 3.31; 1.34, 2.12.2 and 3.31; 1.34, 2.11.6 and 3.33; 1.34, 2.12.1 and 3.33; 1.34, 2.12.2 and 3.33; 1.34, 2.11.6 and 3.34; 1.34, 2.12.1 and 3.34; 1.34, 2.12.2 and 3.34; 1.34, 2.11.6 and 3.35; 1.34, 2.12.1 and 3.35; 1.34, 2.12.2 and 3.35; 1.34, 2.11.6 and 3.36; 1.34, 2.12.1 and 3.36; 1.34, 2.12.2 and 3.36; 1.34, 2.11.6 and 3.37; 1.34, 2.11.6 and 3.38; 1.34, 2.12.1 and 3.37; 1.34, 2.12.1 and 3.38; 1.34, 2.12.2 and 3.37; 1.34, 2.12.2 and 3.38; 1.34, 2.13.3 and 3.1; 1.34, 2.13.4 and 3.1; 1.34, 2.13.3 and 3.3; 1.34, 2.13.4 and 3.3; 1.34, 2.13.3 and 3.4; 1.34, 2.13.4 and 3.4; 1.34, 2.13.3 and 3.8; 1.34, 2.13.4 and 3.8; 1.34, 2.13.3 and 3.9; 1.34, 2.13.4 and 3.9; 1.34, 2.13.3 and 3.16; 1.34, 2.13.4 and 3.16; 1.34, 2.13.3 and 3.17; 1.34, 2.13.4 and 3.17; 1.34, 2.13.3 and 3.20; 1.34, 2.13.4 and 3.20; 1.34, 2.13.3 and 3.24; 1.34, 2.13.4 and 3.24; 1.34, 2.13.3 and 3.25; 1.34, 2.13.4 and 3.25; 1.34, 2.13.3 and 3.26; 1.34, 2.13.4 and 3.26; 1.34, 2.13.3 and 3.27; 1.34, 2.13.4 and 3.27; 1.34, 2.13.3 and 3.28; 1.34, 2.13.4 and 3.28; 1.34, 2.13.3 and 3.29; 1.34, 2.13.4 and 3.29; 1.34, 2.13.3 and 3.30; 1.34, 2.13.4 and 3.30; 1.34, 2.13.3 and 3.31; 1.34, 2.13.4 and 3.31; 1.34, 2.13.3 and 3.33; 1.34, 2.13.4 and 3.33; 1.34, 2.13.3 and 3.34; 1.34, 2.13.4 and 3.34; 1.34, 2.13.3 and 3.35; 1.34, 2.13.4 and 3.35; 1.34, 2.13.3 and 3.36; 1.34, 2.13.4 and 3.36; 1.34, 2.13.3 and 3.37; 1.34, 2.13.3 and 3.38; 1.34, 2.13.4 and 3.37; 1.34, 2.13.4 and 3.38; 1.37, 2.1 and 3.1; 1.37, 2.2 and 3.1; 1.37, 2.3 and 3.1; 1.37, 2.1 and 3.3; 1.37, 2.2 and 3.3; 1.37, 2.3 and 3.3; 1.37, 2.1 and 3.4; 1.37, 2.2 and 3.4; 1.37, 2.3 and 3.4; 1.37, 2.1 and 3.8; 1.37, 2.2 and 3.8; 1.37, 2.3 and 3.8; 1.37, 2.1 and 3.9; 1.37, 2.2 and 3.9; 1.37, 2.3 and 3.9; 1.37, 2.1 and 3.16; 1.37, 2.2 and 3.16; 1.37, 2.3 and 3.16; 1.37, 2.1 and 3.17; 1.37, 2.2 and 3.17; 1.37, 2.3 and 3.17; 1.37, 2.1 and 3.20; 1.37, 2.2 and 3.20; 1.37, 2.3 and 3.20; 1.37, 2.1 and 3.24; 1.37, 2.2 and 3.24; 1.37, 2.3 and 3.24; 1.37, 2.1 and 3.25; 1.37, 2.2 and 3.25; 1.37, 2.3 and 3.25; 1.37, 2.1 and 3.26; 1.37, 2.2 and 3.26; 1.37, 2.3 and 3.26; 1.37, 2.1 and 3.27; 1.37, 2.2 and 3.27; 1.37, 2.3 and 3.27; 1.37, 2.1 and 3.28; 1.37, 2.2 and 3.28; 1.37, 2.3 and 3.28; 1.37, 2.1 and 3.29; 1.37, 2.2 and 3.29; 1.37, 2.3 and 3.29; 1.37, 2.1 and 3.30; 1.37, 2.2 and 3.30; 1.37, 2.3 and 3.30; 1.37, 2.1 and 3.31; 1.37, 2.2 and 3.31; 1.37, 2.3 and 3.31; 1.37, 2.1 and 3.33; 1.37, 2.2 and 3.33; 1.37, 2.3 and 3.33; 1.37, 2.1 and 3.34; 1.37, 2.2 and 3.34; 1.37, 2.3 and 3.34; 1.37, 2.1 and 3.35; 1.37, 2.2 and 3.35; 1.37, 2.3 and 3.35; 1.37, 2.1 and 3.36; 1.37, 2.2 and 3.36; 1.37, 2.3 and 3.36; 1.37, 2.1 and 3.37; 1.37, 2.1 and 3.38; 1.37, 2.2 and 3.37; 1.37, 2.2 and 3.38; 1.37, 2.3 and 3.37; 1.37, 2.3 and 3.38; 1.37, 2.4 and 3.1; 1.37, 2.5 and 3.1; 1.37, 2.7 and 3.1; 1.37, 2.4 and 3.3; 1.37, 2.5 and 3.3; 1.37, 2.7 and 3.3; 1.37, 2.4 and 3.4; 1.37, 2.5 and 3.4; 1.37, 2.7 and 3.4; 1.37, 2.4 and 3.8; 1.37, 2.5 and 3.8; 1.37, 2.7 and 3.8; 1.37, 2.4 and 3.9; 1.37, 2.5 and 3.9; 1.37, 2.7 and 3.9; 1.37, 2.4 and 3.16; 1.37, 2.5 and 3.16; 1.37, 2.7 and 3.16; 1.37, 2.4 and 3.17; 1.37, 2.5 and 3.17; 1.37, 2.7 and 3.17; 1.37, 2.4 and 3.20; 1.37, 2.5 and 3.20; 1.37, 2.7 and 3.20; 1.37, 2.4 and 3.24; 1.37, 2.5 and 3.24; 1.37, 2.7 and 3.24; 1.37, 2.4 and 3.25; 1.37, 2.5 and 3.25; 1.37, 2.7 and 3.25; 1.37, 2.4 and 3.26; 1.37, 2.5 and 3.26; 1.37, 2.7 and 3.26; 1.37, 2.4 and 3.27; 1.37, 2.5 and 3.27; 1.37, 2.7 and 3.27; 1.37, 2.4 and 3.28; 1.37, 2.5 and 3.28; 1.37, 2.7 and 3.28; 1.37, 2.4 and 3.29; 1.37, 2.5 and 3.29; 1.37, 2.7 and 3.29; 1.37, 2.4 and 3.30; 1.37, 2.5 and 3.30; 1.37, 2.7 and 3.30; 1.37, 2.4 and 3.31; 1.37, 2.5 and 3.31; 1.37, 2.7 and 3.31; 1.37, 2.4 and 3.33; 1.37, 2.5 and 3.33; 1.37, 2.7 and 3.33; 1.37, 2.4 and 3.34; 1.37, 2.5 and 3.34; 1.37, 2.7 and 3.34; 1.37, 2.4 and 3.35; 1.37, 2.5 and 3.35; 1.37, 2.7 and 3.35; 1.37, 2.4 and 3.36; 1.37, 2.5 and 3.36; 1.37, 2.7 and 3.36; 1.37, 2.4 and 3.37; 1.37, 2.4 and 3.38; 1.37, 2.5 and 3.37; 1.37, 2.5 and 3.38; 1.37, 2.7 and 3.37; 1.37, 2.7 and 3.38; 1.37, 2.9.1 and 3.1; 1.37, 2.9.2 and 3.1; 1.37, 2.9.1 and 3.3; 1.37, 2.9.2 and 3.3; 1.37, 2.9.1 and 3.4; 1.37, 2.9.2 and 3.4; 1.37, 2.9.1 and 3.8; 1.37, 2.9.2 and 3.8; 1.37, 2.9.1 and 3.9; 1.37, 2.9.2 and 3.9; 1.37, 2.9.1 and 3.16; 1.37, 2.9.2 and 3.16; 1.37, 2.9.1 and 3.17; 1.37, 2.9.2 and 3.17; 1.37, 2.9.1 and 3.20; 1.37, 2.9.2 and 3.20; 1.37, 2.9.1 and 3.24; 1.37, 2.9.2 and 3.24; 1.37, 2.9.1 and 3.25; 1.37, 2.9.2 and 3.25; 1.37, 2.9.1 and 3.26; 1.37, 2.9.2 and 3.26; 1.37, 2.9.1 and 3.27; 1.37, 2.9.2 and 3.27; 1.37, 2.9.1 and 3.28; 1.37, 2.9.2 and 3.28; 1.37, 2.9.1 and 3.29; 1.37, 2.9.2 and 3.29; 1.37, 2.9.1 and 3.30; 1.37, 2.9.2 and 3.30; 1.37, 2.9.1 and 3.31; 1.37, 2.9.2 and 3.31; 1.37, 2.9.1 and 3.33; 1.37, 2.9.2 and 3.33; 1.37, 2.9.1 and 3.34; 1.37, 2.9.2 and 3.34; 1.37, 2.9.1 and 3.35; 1.37, 2.9.2 and 3.35; 1.37, 2.9.1 and 3.36; 1.37, 2.9.2 and 3.36; 1.37, 2.9.1 and 3.37; 1.37, 2.9.1 and 3.38; 1.37, 2.9.2 and 3.37; 1.37, 2.9.2 and 3.38; 1.37, 2.10.1 and 3.1; 1.37, 2.10.2 and 3.1; 1.37, 2.10.1 and 3.3; 1.37, 2.10.2 and 3.3; 1.37, 2.10.1 and 3.4; 1.37, 2.10.2 and 3.4; 1.37, 2.10.1 and 3.8; 1.37, 2.10.2 and 3.8; 1.37, 2.10.1 and 3.9; 1.37, 2.10.2 and 3.9; 1.37, 2.10.1 and 3.16; 1.37, 2.10.2 and 3.16; 1.37, 2.10.1 and 3.17; 1.37, 2.10.2 and 3.17; 1.37, 2.10.1 and 3.20; 1.37, 2.10.2 and 3.20; 1.37, 2.10.1 and 3.24; 1.37, 2.10.2 and 3.24; 1.37, 2.10.1 and 3.25; 1.37, 2.10.2 and 3.25; 1.37, 2.10.1 and 3.26; 1.37, 2.10.2 and 3.26; 1.37, 2.10.1 and 3.27; 1.37, 2.10.2 and 3.27; 1.37, 2.10.1 and 3.28; 1.37, 2.10.2 and 3.28; 1.37, 2.10.1 and 3.29; 1.37, 2.10.2 and 3.29; 1.37, 2.10.1 and 3.30; 1.37, 2.10.2 and 3.30; 1.37, 2.10.1 and 3.31; 1.37, 2.10.2 and 3.31; 1.37, 2.10.1 and 3.33; 1.37, 2.10.2 and 3.33; 1.37, 2.10.1 and 3.34; 1.37, 2.10.2 and 3.34; 1.37, 2.10.1 and 3.35; 1.37, 2.10.2 and 3.35; 1.37, 2.10.1 and 3.36; 1.37, 2.10.2 and 3.36; 1.37, 2.10.1 and 3.37; 1.37, 2.10.1 and 3.38; 1.37, 2.10.2 and 3.37; 1.37, 2.10.2 and 3.38; 1.37, 2.11.5 and 3.1; 1.37, 2.11.5 and 3.3; 1.37, 2.11.5 and 3.4; 1.37, 2.11.5 and 3.8; 1.37, 2.11.5 and 3.9; 1.37, 2.11.5 and 3.16; 1.37, 2.11.5 and 3.17; 1.37, 2.11.5 and 3.20; 1.37, 2.11.5 and 3.24; 1.37, 2.11.5 and 3.25; 1.37, 2.11.5 and 3.26; 1.37, 2.11.5 and 3.27; 1.37, 2.11.5 and 3.28; 1.37, 2.11.5 and 3.29; 1.37, 2.11.5 and 3.30; 1.37, 2.11.5 and 3.31; 1.37, 2.11.5 and 3.33; 1.37, 2.11.5 and 3.34; 1.37, 2.11.5 and 3.35; 1.37, 2.11.5 and 3.36; 1.37, 2.11.5 and 3.37; 1.37, 2.11.5 and 3.38; 1.37, 2.11.6 and 3.1; 1.37, 2.12.1 and 3.1; 1.37, 2.12.2 and 3.1; 1.37, 2.11.6 and 3.3; 1.37, 2.12.1 and 3.3; 1.37, 2.12.2 and 3.3; 1.37, 2.11.6 and 3.4; 1.37, 2.12.1 and 3.4; 1.37, 2.12.2 and 3.4; 1.37, 2.11.6 and 3.8; 1.37, 2.12.1 and 3.8; 1.37, 2.12.2 and 3.8; 1.37, 2.11.6 and 3.9; 1.37, 2.12.1 and 3.9; 1.37, 2.12.2 and 3.9; 1.37, 2.11.6 and 3.16; 1.37, 2.12.1 and 3.16; 1.37, 2.12.2 and 3.16; 1.37, 2.11.6 and 3.17; 1.37, 2.12.1 and 3.17; 1.37, 2.12.2 and 3.17; 1.37, 2.11.6 and 3.20; 1.37, 2.12.1 and 3.20; 1.37, 2.12.2 and 3.20; 1.37, 2.11.6 and 3.24; 1.37, 2.12.1 and 3.24; 1.37, 2.12.2 and 3.24; 1.37, 2.11.6 and 3.25; 1.37, 2.12.1 and 3.25; 1.37, 2.12.2 and 3.25; 1.37, 2.11.6 and 3.26; 1.37, 2.12.1 and 3.26; 1.37, 2.12.2 and 3.26; 1.37, 2.11.6 and 3.27; 1.37, 2.12.1 and 3.27; 1.37, 2.12.2 and 3.27; 1.37, 2.11.6 and 3.28; 1.37, 2.12.1 and 3.28; 1.37, 2.12.2 and 3.28; 1.37, 2.11.6 and 3.29; 1.37, 2.12.1 and 3.29; 1.37, 2.12.2 and 3.29; 1.37, 2.11.6 and 3.30; 1.37, 2.12.1 and 3.30; 1.37, 2.12.2 and 3.30; 1.37, 2.11.6 and 3.31; 1.37, 2.12.1 and 3.31; 1.37, 2.12.2 and 3.31; 1.37, 2.11.6 and 3.33; 1.37, 2.12.1 and 3.33; 1.37, 2.12.2 and 3.33; 1.37, 2.11.6 and 3.34; 1.37, 2.12.1 and 3.34; 1.37, 2.12.2 and 3.34; 1.37, 2.11.6 and 3.35; 1.37, 2.12.1 and 3.35; 1.37, 2.12.2 and 3.35; 1.37, 2.11.6 and 3.36; 1.37, 2.12.1 and 3.36; 1.37, 2.12.2 and 3.36; 1.37, 2.11.6 and 3.37; 1.37, 2.11.6 and 3.38; 1.37, 2.12.1 and 3.37; 1.37, 2.12.1 and 3.38; 1.37, 2.12.2 and 3.37; 1.37, 2.12.2 and 3.38; 1.37, 2.13.3 and 3.1; 1.37, 2.13.4 and 3.1; 1.37, 2.13.3 and 3.3; 1.37, 2.13.4 and 3.3; 1.37, 2.13.3 and 3.4; 1.37, 2.13.4 and 3.4; 1.37, 2.13.3 and 3.8; 1.37, 2.13.4 and 3.8; 1.37, 2.13.3 and 3.9; 1.37, 2.13.4 and 3.9; 1.37, 2.13.3 and 3.16; 1.37, 2.13.4 and 3.16; 1.37, 2.13.3 and 3.17; 1.37, 2.13.4 and 3.17; 1.37, 2.13.3 and 3.20; 1.37, 2.13.4 and 3.20; 1.37, 2.13.3 and 3.24; 1.37, 2.13.4 and 3.24; 1.37, 2.13.3 and 3.25; 1.37, 2.13.4 and 3.25; 1.37, 2.13.3 and 3.26; 1.37, 2.13.4 and 3.26; 1.37, 2.13.3 and 3.27; 1.37, 2.13.4 and 3.27; 1.37, 2.13.3 and 3.28; 1.37, 2.13.4 and 3.28; 1.37, 2.13.3 and 3.29; 1.37, 2.13.4 and 3.29; 1.37, 2.13.3 and 3.30; 1.37, 2.13.4 and 3.30; 1.37, 2.13.3 and 3.31; 1.37, 2.13.4 and 3.31; 1.37, 2.13.3 and 3.33; 1.37, 2.13.4 and 3.33; 1.37, 2.13.3 and 3.34; 1.37, 2.13.4 and 3.34; 1.37, 2.13.3 and 3.35; 1.37, 2.13.4 and 3.35; 1.37, 2.13.3 and 3.36; 1.37, 2.13.4 and 3.36; 1.37, 2.13.3 and 3.37; 1.37, 2.13.3 and 3.38; 1.37, 2.13.4 and 3.37; 1.37, 2.13.4 and 3.38; 1.38, 2.1 and 3.1; 1.38, 2.2 and 3.1; 1.38, 2.3 and 3.1; 1.38, 2.1 and 3.3; 1.38, 2.2 and 3.3; 1.38, 2.3 and 3.3; 1.38, 2.1 and 3.4; 1.38, 2.2 and 3.4; 1.38, 2.3 and 3.4; 1.38, 2.1 and 3.8; 1.38, 2.2 and 3.8; 1.38, 2.3 and 3.8; 1.38, 2.1 and 3.9; 1.38, 2.2 and 3.9; 1.38, 2.3 and 3.9; 1.38, 2.1 and 3.16; 1.38, 2.2 and 3.16; 1.38, 2.3 and 3.16; 1.38, 2.1 and 3.17; 1.38, 2.2 and 3.17; 1.38, 2.3 and 3.17; 1.38, 2.1 and 3.20; 1.38, 2.2 and 3.20; 1.38, 2.3 and 3.20; 1.38, 2.1 and 3.24; 1.38, 2.2 and 3.24; 1.38, 2.3 and 3.24; 1.38, 2.1 and 3.25; 1.38, 2.2 and 3.25; 1.38, 2.3 and 3.25; 1.38, 2.1 and 3.26; 1.38, 2.2 and 3.26; 1.38, 2.3 and 3.26; 1.38, 2.1 and 3.27; 1.38, 2.2 and 3.27; 1.38, 2.3 and 3.27; 1.38, 2.1 and 3.28; 1.38, 2.2 and 3.28; 1.38, 2.3 and 3.28; 1.38, 2.1 and 3.29; 1.38, 2.2 and 3.29; 1.38, 2.3 and 3.29; 1.38, 2.1 and 3.30; 1.38, 2.2 and 3.30; 1.38, 2.3 and 3.30; 1.38, 2.1 and 3.31; 1.38, 2.2 and 3.31; 1.38, 2.3 and 3.31; 1.38, 2.1 and 3.33; 1.38, 2.2 and 3.33; 1.38, 2.3 and 3.33; 1.38, 2.1 and 3.34; 1.38, 2.2 and 3.34; 1.38, 2.3 and 3.34; 1.38, 2.1 and 3.35; 1.38, 2.2 and 3.35; 1.38, 2.3 and 3.35; 1.38, 2.1 and 3.36; 1.38, 2.2 and 3.36; 1.38, 2.3 and 3.36; 1.38, 2.1 and 3.37; 1.38, 2.1 and 3.38; 1.38, 2.2 and 3.37; 1.38, 2.2 and 3.38; 1.38, 2.3 and 3.37; 1.38, 2.3 and 3.38; 1.38, 2.4 and 3.1; 1.38, 2.5 and 3.1; 1.38, 2.7 and 3.1; 1.38, 2.4 and 3.3; 1.38, 2.5 and 3.3; 1.38, 2.7 and 3.3; 1.38, 2.4 and 3.4; 1.38, 2.5 and 3.4; 1.38, 2.7 and 3.4; 1.38, 2.4 and 3.8; 1.38, 2.5 and 3.8; 1.38, 2.7 and 3.8; 1.38, 2.4 and 3.9; 1.38, 2.5 and 3.9; 1.38, 2.7 and 3.9; 1.38, 2.4 and 3.16; 1.38, 2.5 and 3.16; 1.38, 2.7 and 3.16; 1.38, 2.4 and 3.17; 1.38, 2.5 and 3.17; 1.38, 2.7 and 3.17; 1.38, 2.4 and 3.20; 1.38, 2.5 and 3.20; 1.38, 2.7 and 3.20; 1.38, 2.4 and 3.24; 1.38, 2.5 and 3.24; 1.38, 2.7 and 3.24; 1.38, 2.4 and 3.25; 1.38, 2.5 and 3.25; 1.38, 2.7 and 3.25; 1.38, 2.4 and 3.26; 1.38, 2.5 and 3.26; 1.38, 2.7 and 3.26; 1.38, 2.4 and 3.27; 1.38, 2.5 and 3.27; 1.38, 2.7 and 3.27; 1.38, 2.4 and 3.28; 1.38, 2.5 and 3.28; 1.38, 2.7 and 3.28; 1.38, 2.4 and 3.29; 1.38, 2.5 and 3.29; 1.38, 2.7 and 3.29; 1.38, 2.4 and 3.30; 1.38, 2.5 and 3.30; 1.38, 2.7 and 3.30; 1.38, 2.4 and 3.31; 1.38, 2.5 and 3.31; 1.38, 2.7 and 3.31; 1.38, 2.4 and 3.33; 1.38, 2.5 and 3.33; 1.38, 2.7 and 3.33; 1.38, 2.4 and 3.34; 1.38, 2.5 and 3.34; 1.38, 2.7 and 3.34; 1.38, 2.4 and 3.35; 1.38, 2.5 and 3.35; 1.38, 2.7 and 3.35; 1.38, 2.4 and 3.36; 1.38, 2.5 and 3.36; 1.38, 2.7 and 3.36; 1.38, 2.4 and 3.37; 1.38, 2.4 and 3.38; 1.38, 2.5 and 3.37; 1.38, 2.5 and 3.38; 1.38, 2.7 and 3.37; 1.38, 2.7 and 3.38; 1.38, 2.9.1 and 3.1; 1.38, 2.9.2 and 3.1; 1.38, 2.9.1 and 3.3; 1.38, 2.9.2 and 3.3; 1.38, 2.9.1 and 3.4; 1.38, 2.9.2 and 3.4; 1.38, 2.9.1 and 3.8; 1.38, 2.9.2 and 3.8; 1.38, 2.9.1 and 3.9; 1.38, 2.9.2 and 3.9; 1.38, 2.9.1 and 3.16; 1.38, 2.9.2 and 3.16, 1.38, 2.9.1 and 3.17; 1.38, 2.9.2 and 3.17; 1.38, 2.9.1 and 3.20; 1.38, 2.9.2 and 3.20; 1.38, 2.9.1 and 3.24; 1.38, 2.9.2 and 3.24; 1.38, 2.9.1 and 3.25; 1.38, 2.9.2 and 3.25, 1.38, 2.9.1 and 3.26; 1.38, 2.9.2 and 3.26; 1.38, 2.9.1 and 3.27; 1.38, 2.9.2 and 3.27; 1.38, 2.9.1 and 3.28; 1.38, 2.9.2 and 3.28; 1.38, 2.9.1 and 3.29; 1.38, 2.9.2 and 3.29; 1.38, 2.9.1 and 3.30, 1.38, 2.9.2 and 3.30; 1.38, 2.9.1 and 3.31; 1.38, 2.9.2 and 3.31; 1.38, 2.9.1 and 3.33; 1.38, 2.9.2 and 3.33; 1.38, 2.9.1 and 3.34; 1.38, 2.9.2 and 3.34; 1.38, 2.9.1 and 3.35; 1.38, 2.9.2 and 3.35, 1.38, 2.9.1 and 3.36; 1.38, 2.9.2 and 3.36; 1.38, 2.9.1 and 3.37; 1.38, 2.9.1 and 3.38; 1.38, 2.9.2 and 3.37; 1.38, 2.9.2 and 3.38; 1.38, 2.10.1 and 3.1; 1.38, 2.10.2 and 3.1, 1.38, 2.10.1 and 3.3; 1.38, 2.10.2 and 3.3; 1.38, 2.10.1 and 3.4; 1.38, 2.10.2 and 3.4, 1.38, 2.10.1 and 3.8; 1.38, 2.10.2 and 3.8; 1.38, 2.10.1 and 3.9; 1.38, 2.10.2 and 3.9, 1.38, 2.10.1 and 3.16; 1.38, 2.10.2 and 3.16; 1.38, 2.10.1 and 3.17; 1.38, 2.10.2 and 3.17, 1.38, 2.10.1 and 3.20; 1.38, 2.10.2 and 3.20; 1.38, 2.10.1 and 3.24; 1.38, 2.10.2 and 3.24, 1.38, 2.10.1 and 3.25, 1.38, 2.10.2 and 3.25; 1.38, 2.10.1 and 3.26; 1.38, 2.10.2 and 3.26, 1.38, 2.10.1 and 3.27; 1.38, 2.10.2 and 3.27; 1.38, 2.10.1 and 3.28; 1.38, 2.10.2 and 3.28, 1.38, 2.10.1 and 3.29; 1.38, 2.10.2 and 3.29; 1.38, 2.10.1 and 3.30; 1.38, 2.10.2 and 3.30, 1.38, 2.10.1 and 3.31; 1.38, 2.10.2 and 3.31; 1.38, 2.10.1 and 3.33; 1.38, 2.10.2 and 3.33, 1.38, 2.10.1 and 3.34; 1.38, 2.10.2 and 3.34; 1.38, 2.10.1 and 3.35; 1.38, 2.10.2 and 3.35; 1.38, 2.10.1 and 3.36; 1.38, 2.10.2 and 3.36; 1.38, 2.10.1 and 3.37; 1.38, 2.10.1 and 3.38; 1.38, 2.10.2 and 3.37; 1.38, 2.10.2 and 3.38; 1.38, 2.11.5 and 3.1; 1.38, 2.11.5 and 3.3; 1.38, 2.11.5 and 3.4; 1.38, 2.11.5 and 3.8; 1.38, 2.11.5 and 3.9; 1.38, 2.11.5 and 3.16; 1.38, 2.11.5 and 3.17; 1.38, 2.11.5 and 3.20; 1.38, 2.11.5 and 3.24; 1.38, 2.11.5 and 3.25; 1.38, 2.11.5 and 3.26; 1.38, 2.11.5 and 3.27; 1.38, 2.11.5 and 3.28; 1.38, 2.11.5 and 3.29; 1.38, 2.11.5 and 3.30; 1.38, 2.11.5 and 3.31; 1.38, 2.11.5 and 3.33; 1.38, 2.11.5 and 3.34; 1.38, 2.11.5 and 3.35; 1.38, 2.11.5 and 3.36; 1.38, 2.11.5 and 3.37; 1.38, 2.11.5 and 3.38; 1.38, 2.11.6 and 3.1; 1.38, 2.12.1 and 3.1; 1.38, 2.12.2 and 3.1; 1.38, 2.11.6 and 3.3; 1.38, 2.12.1 and 3.3; 1.38, 2.12.2 and 3.3; 1.38, 2.11.6 and 3.4; 1.38, 2.12.1 and 3.4; 1.38, 2.12.2 and 3.4; 1.38, 2.11.6 and 3.8; 1.38, 2.12.1 and 3.8; 1.38, 2.12.2 and 3.8; 1.38, 2.11.6 and 3.9; 1.38, 2.12.1 and 3.9; 1.38, 2.12.2 and 3.9; 1.38, 2.11.6 and 3.16; 1.38, 2.12.1 and 3.16; 1.38, 2.12.2 and 3.16; 1.38, 2.11.6 and 3.17; 1.38, 2.12.1 and 3.17; 1.38, 2.12.2 and 3.17; 1.38, 2.11.6 and 3.20; 1.38, 2.12.1 and 3.20; 1.38, 2.12.2 and 3.20; 1.38, 2.11.6 and 3.24; 1.38, 2.12.1 and 3.24; 1.38, 2.12.2 and 3.24; 1.38, 2.11.6 and 3.25; 1.38, 2.12.1 and 3.25; 1.38, 2.12.2 and 3.25; 1.38, 2.11.6 and 3.26; 1.38, 2.12.1 and 3.26; 1.38, 2.12.2 and 3.26; 1.38, 2.11.6 and 3.27; 1.38, 2.12.1 and 3.27; 1.38, 2.12.2 and 3.27; 1.38, 2.11.6 and 3.28; 1.38, 2.12.1 and 3.28; 1.38, 2.12.2 and 3.28; 1.38, 2.11.6 and 3.29; 1.38, 2.12.1 and 3.29; 1.38, 2.12.2 and 3.29; 1.38, 2.11.6 and 3.30; 1.38, 2.12.1 and 3.30; 1.38, 2.12.2 and 3.30; 1.38, 2.11.6 and 3.31; 1.38, 2.12.1 and 3.31; 1.38, 2.12.2 and 3.31; 1.38, 2.11.6 and 3.33; 1.38, 2.12.1 and 3.33; 1.38, 2.12.2 and 3.33; 1.38, 2.11.6 and 3.34; 1.38, 2.12.1 and 3.34; 1.38, 2.12.2 and 3.34; 1.38, 2.11.6 and 3.35; 1.38, 2.12.1 and 3.35; 1.38, 2.12.2 and 3.35; 1.38, 2.11.6 and 3.36; 1.38, 2.12.1 and 3.36; 1.38, 2.12.2 and 3.36; 1.38, 2.11.6 and 3.37; 1.38, 2.11.6 and 3.38; 1.38, 2.12.1 and 3.37; 1.38, 2.12.1 and 3.38; 1.38, 2.12.2 and 3.37; 1.38, 2.12.2 and 3.38; 1.38, 2.13.3 and 3.1; 1.38, 2.13.4 and 3.1; 1.38, 2.13.3 and 3.3; 1.38, 2.13.4 and 3.3; 1.38, 2.13.3 and 3.4; 1.38, 2.13.4 and 3.4; 1.38, 2.13.3 and 3.8; 1.38, 2.13.4 and 3.8; 1.38, 2.13.3 and 3.9; 1.38, 2.13.4 and 3.9; 1.38, 2.13.3 and 3.16; 1.38, 2.13.4 and 3.16; 1.38, 2.13.3 and 3.17; 1.38, 2.13.4 and 3.17; 1.38, 2.13.3 and 3.20; 1.38, 2.13.4 and 3.20; 1.38, 2.13.3 and 3.24; 1.38, 2.13.4 and 3.24; 1.38, 2.13.3 and 3.25; 1.38, 2.13.4 and 3.25; 1.38, 2.13.3 and 3.26; 1.38, 2.13.4 and 3.26; 1.38, 2.13.3 and 3.27; 1.38, 2.13.4 and 3.27; 1.38, 2.13.3 and 3.28; 1.38, 2.13.4 and 3.28; 1.38, 2.13.3 and 3.29; 1.38, 2.13.4 and 3.29; 1.38, 2.13.3 and 3.30; 1.38, 2.13.4 and 3.30; 1.38, 2.13.3 and 3.31; 1.38, 2.13.4 and 3.31; 1.38, 2.13.3 and 3.33; 1.38, 2.13.4 and 3.33; 1.38, 2.13.3 and 3.34; 1.38, 2.13.4 and 3.34; 1.38, 2.13.3 and 3.35; 1.38, 2.13.4 and 3.35; 1.38, 2.13.3 and 3.36; 1.38, 2.13.4 and 3.36; 1.38, 2.13.3 and 3.37; 1.38, 2.13.3 and 3.38; 1.38, 2.13.4 and 3.37; 1.38, 2.13.4 and 3.38; 1.39, 2.1 and 3.1; 1.39, 2.2 and 3.1; 1.39, 2.3 and 3.1; 1.39, 2.1 and 3.3; 1.39, 2.2 and 3.3; 1.39, 2.3 and 3.3; 1.39, 2.1 and 3.4; 1.39, 2.2 and 3.4; 1.39, 2.3 and 3.4; 1.39, 2.1 and 3.8; 1.39, 2.2 and 3.8; 1.39, 2.3 and 3.8; 1.39, 2.1 and 3.9; 1.39, 2.2 and 3.9; 1.39, 2.3 and 3.9; 1.39, 2.1 and 3.16; 1.39, 2.2 and 3.16; 1.39, 2.3 and 3.16; 1.39, 2.1 and 3.17; 1.39, 2.2 and 3.17; 1.39, 2.3 and 3.17; 1.39, 2.1 and 3.20; 1.39, 2.2 and 3.20; 1.39, 2.3 and 3.20; 1.39, 2.1 and 3.24; 1.39, 2.2 and 3.24; 1.39, 2.3 and 3.24; 1.39, 2.1 and 3.25; 1.39, 2.2 and 3.25; 1.39, 2.3 and 3.25; 1.39, 2.1 and 3.26; 1.39, 2.2 and 3.26; 1.39, 2.3 and 3.26; 1.39, 2.1 and 3.27; 1.39, 2.2 and 3.27; 1.39, 2.3 and 3.27; 1.39, 2.1 and 3.28; 1.39, 2.2 and 3.28; 1.39, 2.3 and 3.28; 1.39, 2.1 and 3.29; 1.39, 2.2 and 3.29; 1.39, 2.3 and 3.29; 1.39, 2.1 and 3.30; 1.39, 2.2 and 3.30; 1.39, 2.3 and 3.30; 1.39, 2.1 and 3.31; 1.39, 2.2 and 3.31; 1.39, 2.3 and 3.31; 1.39, 2.1 and 3.33; 1.39, 2.2 and 3.33; 1.39, 2.3 and 3.33; 1.39, 2.1 and 3.34; 1.39, 2.2 and 3.34; 1.39, 2.3 and 3.34; 1.39, 2.1 and 3.35; 1.39, 2.2 and 3.35; 1.39, 2.3 and 3.35; 1.39, 2.1 and 3.36; 1.39, 2.2 and 3.36; 1.39, 2.3 and 3.36; 1.39, 2.1 and 3.37; 1.39, 2.1 and 3.38; 1.39, 2.2 and 3.37; 1.39, 2.2 and 3.38; 1.39, 2.3 and 3.37; 1.39, 2.3 and 3.38; 1.39, 2.4 and 3.1; 1.39, 2.5 and 3.1; 1.39, 2.7 and 3.1; 1.39, 2.4 and 3.3; 1.39, 2.5 and 3.3; 1.39, 2.7 and 3.3; 1.39, 2.4 and 3.4; 1.39, 2.5 and 3.4; 1.39, 2.7 and 3.4; 1.39, 2.4 and 3.8; 1.39, 2.5 and 3.8; 1.39, 2.7 and 3.8; 1.39, 2.4 and 3.9; 1.39, 2.5 and 3.9; 1.39, 2.7 and 3.9; 1.39, 2.4 and 3.16; 1.39, 2.5 and 3.16; 1.39, 2.7 and 3.16; 1.39, 2.4 and 3.17; 1.39, 2.5 and 3.17; 1.39, 2.7 and 3.17; 1.39, 2.4 and 3.20; 1.39, 2.5 and 3.20; 1.39, 2.7 and 3.20; 1.39, 2.4 and 3.24; 1.39, 2.5 and 3.24; 1.39, 2.7 and 3.24; 1.39, 2.4 and 3.25; 1.39, 2.5 and 3.25; 1.39, 2.7 and 3.25; 1.39, 2.4 and 3.26; 1.39, 2.5 and 3.26; 1.39, 2.7 and 3.26; 1.39, 2.4 and 3.27; 1.39, 2.5 and 3.27; 1.39, 2.7 and 3.27; 1.39, 2.4 and 3.28; 1.39, 2.5 and 3.28; 1.39, 2.7 and 3.28; 1.39, 2.4 and 3.29; 1.39, 2.5 and 3.29; 1.39, 2.7 and 3.29; 1.39, 2.4 and 3.30; 1.39, 2.5 and 3.30; 1.39, 2.7 and 3.30; 1.39, 2.4 and 3.31; 1.39, 2.5 and 3.31; 1.39, 2.7 and 3.31; 1.39, 2.4 and 3.33; 1.39, 2.5 and 3.33; 1.39, 2.7 and 3.33; 1.39, 2.4 and 3.34; 1.39, 2.5 and 3.34; 1.39, 2.7 and 3.34; 1.39, 2.4 and 3.35; 1.39, 2.5 and 3.35; 1.39, 2.7 and 3.35; 1.39, 2.4 and 3.36; 1.39, 2.5 and 3.36; 1.39, 2.7 and 3.36; 1.39, 2.4 and 3.37; 1.39, 2.4 and 3.38; 1.39, 2.5 and 3.37; 1.39, 2.5 and 3.38; 1.39, 2.7 and 3.37; 1.39, 2.7 and 3.38; 1.39, 2.9.1 and 3.1; 1.39, 2.9.2 and 3.1; 1.39, 2.9.1 and 3.3; 1.39, 2.9.2 and 3.3; 1.39, 2.9.1 and 3.4; 1.39, 2.9.2 and 3.4; 1.39, 2.9.1 and 3.8; 1.39, 2.9.2 and 3.8; 1.39, 2.9.1 and 3.9; 1.39, 2.9.2 and 3.9; 1.39, 2.9.1 and 3.16; 1.39, 2.9.2 and 3.16; 1.39, 2.9.1 and 3.17; 1.39, 2.9.2 and 3.17; 1.39, 2.9.1 and 3.20; 1.39, 2.9.2 and 3.20; 1.39, 2.9.1 and 3.24; 1.39, 2.9.2 and 3.24; 1.39, 2.9.1 and 3.25; 1.39, 2.9.2 and 3.25; 1.39, 2.9.1 and 3.26; 1.39, 2.9.2 and 3.26; 1.39, 2.9.1 and 3.27; 1.39, 2.9.2 and 3.27; 1.39, 2.9.1 and 3.28; 1.39, 2.9.2 and 3.28; 1.39, 2.9.1 and 3.29; 1.39, 2.9.2 and 3.29; 1.39, 2.9.1 and 3.30; 1.39, 2.9.2 and 3.30; 1.39, 2.9.1 and 3.31; 1.39, 2.9.2 and 3.31; 1.39, 2.9.1 and 3.33; 1.39, 2.9.2 and 3.33; 1.39, 2.9.1 and 3.34; 1.39, 2.9.2 and 3.34; 1.39, 2.9.1 and 3.35; 1.39, 2.9.2 and 3.35; 1.39, 2.9.1 and 3.36; 1.39, 2.9.2 and 3.36; 1.39, 2.9.1 and 3.37; 1.39, 2.9.1 and 3.38; 1.39, 2.9.2 and 3.37; 1.39, 2.9.2 and 3.38; 1.39, 2.10.1 and 3.1; 1.39, 2.10.2 and 3.1; 1.39, 2.10.1 and 3.3; 1.39, 2.10.2 and 3.3; 1.39, 2.10.1 and 3.4; 1.39, 2.10.2 and 3.4; 1.39, 2.10.1 and 3.8; 1.39, 2.10.2 and 3.8; 1.39, 2.10.1 and 3.9; 1.39, 2.10.2 and 3.9; 1.39, 2.10.1 and 3.16; 1.39, 2.10.2 and 3.16; 1.39, 2.10.1 and 3.17; 1.39, 2.10.2 and 3.17; 1.39, 2.10.1 and 3.20; 1.39, 2.10.2 and 3.20; 1.39, 2.10.1 and 3.24; 1.39, 2.10.2 and 3.24; 1.39, 2.10.1 and 3.25; 1.39, 2.10.2 and 3.25; 1.39, 2.10.1 and 3.26; 1.39, 2.10.2 and 3.26; 1.39, 2.10.1 and 3.27; 1.39, 2.10.2 and 3.27; 1.39, 2.10.1 and 3.28; 1.39, 2.10.2 and 3.28; 1.39, 2.10.1 and 3.29; 1.39, 2.10.2 and 3.29; 1.39, 2.10.1 and 3.30; 1.39, 2.10.2 and 3.30; 1.39, 2.10.1 and 3.31; 1.39, 2.10.2 and 3.31; 1.39, 2.10.1 and 3.33; 1.39, 2.10.2 and 3.33; 1.39, 2.10.1 and 3.34; 1.39, 2.10.2 and 3.34; 1.39, 2.10.1 and 3.35; 1.39, 2.10.2 and 3.35; 1.39, 2.10.1 and 3.36; 1.39, 2.10.2 and 3.36; 1.39, 2.10.1 and 3.37; 1.39, 2.10.1 and 3.38; 1.39, 2.10.2 and 3.37; 1.39, 2.10.2 and 3.38; 1.39, 2.11.5 and 3.1; 1.39, 2.11.5 and 3.3; 1.39, 2.11.5 and 3.4; 1.39, 2.11.5 and 3.8; 1.39, 2.11.5 and 3.9; 1.39, 2.11.5 and 3.16; 1.39, 2.11.5 and 3.17; 1.39, 2.11.5 and 3.20; 1.39, 2.11.5 and 3.24; 1.39, 2.11.5 and 3.25; 1.39, 2.11.5 and 3.26; 1.39, 2.11.5 and 3.27; 1.39, 2.11.5 and 3.28; 1.39, 2.11.5 and 3.29; 1.39, 2.11.5 and 3.30; 1.39, 2.11.5 and 3.31; 1.39, 2.11.5 and 3.33; 1.39, 2.11.5 and 3.34; 1.39, 2.11.5 and 3.35; 1.39, 2.11.5 and 3.36; 1.39, 2.11.5 and 3.37; 1.39, 2.11.5 and 3.38; 1.39, 2.11.6 and 3.1; 1.39, 2.12.1 and 3.1; 1.39, 2.12.2 and 3.1; 1.39, 2.11.6 and 3.3; 1.39, 2.12.1 and 3.3; 1.39, 2.12.2 and 3.3; 1.39, 2.11.6 and 3.4; 1.39, 2.12.1 and 3.4; 1.39, 2.12.2 and 3.4; 1.39, 2.11.6 and 3.8; 1.39, 2.12.1 and 3.8; 1.39, 2.12.2 and 3.8; 1.39, 2.11.6 and 3.9; 1.39, 2.12.1 and 3.9; 1.39, 2.12.2 and 3.9; 1.39, 2.11.6 and 3.16; 1.39, 2.12.1 and 3.16; 1.39, 2.12.2 and 3.16; 1.39, 2.11.6 and 3.17; 1.39, 2.12.1 and 3.17; 1.39, 2.12.2 and 3.17; 1.39, 2.11.6 and 3.20; 1.39, 2.12.1 and 3.20; 1.39, 2.12.2 and 3.20; 1.39, 2.11.6 and 3.24; 1.39, 2.12.1 and 3.24; 1.39, 2.12.2 and 3.24; 1.39, 2.11.6 and 3.25; 1.39, 2.12.1 and 3.25; 1.39, 2.12.2 and 3.25; 1.39, 2.11.6 and 3.26; 1.39, 2.12.1 and 3.26; 1.39, 2.12.2 and 3.26; 1.39, 2.11.6 and 3.27; 1.39, 2.12.1 and 3.27; 1.39, 2.12.2 and 3.27; 1.39, 2.11.6 and 3.28; 1.39, 2.12.1 and 3.28; 1.39, 2.12.2 and 3.28; 1.39, 2.11.6 and 3.29; 1.39, 2.12.1 and 3.29; 1.39, 2.12.2 and 3.29; 1.39, 2.11.6 and 3.30; 1.39, 2.12.1 and 3.30; 1.39, 2.12.2 and 3.30; 1.39, 2.11.6 and 3.31; 1.39, 2.12.1 and 3.31; 1.39, 2.12.2 and 3.31; 1.39, 2.11.6 and 3.33; 1.39, 2.12.1 and 3.33; 1.39, 2.12.2 and 3.33; 1.39, 2.11.6 and 3.34; 1.39, 2.12.1 and 3.34; 1.39, 2.12.2 and 3.34; 1.39, 2.11.6 and 3.35; 1.39, 2.12.1 and 3.35; 1.39, 2.12.2 and 3.35; 1.39, 2.11.6 and 3.36; 1.39, 2.12.1 and 3.36; 1.39, 2.12.2 and 3.36; 1.39, 2.11.6 and 3.37; 1.39, 2.11.6 and 3.38; 1.39, 2.12.1 and 3.37; 1.39, 2.12.1 and 3.38; 1.39, 2.12.2 and 3.37; 1.39, 2.12.2 and 3.38; 1.39, 2.13.3 and 3.1; 1.39, 2.13.4 and 3.1; 1.39, 2.13.3 and 3.3; 1.39, 2.13.4 and 3.3; 1.39, 2.13.3 and 3.4; 1.39, 2.13.4 and 3.4; 1.39, 2.13.3 and 3.8; 1.39, 2.13.4 and 3.8; 1.39, 2.13.3 and 3.9; 1.39, 2.13.4 and 3.9; 1.39, 2.13.3 and 3.16; 1.39, 2.13.4 and 3.16; 1.39, 2.13.3 and 3.17; 1.39, 2.13.4 and 3.17; 1.39, 2.13.3 and 3.20; 1.39, 2.13.4 and 3.20; 1.39, 2.13.3 and 3.24; 1.39, 2.13.4 and 3.24; 1.39, 2.13.3 and 3.25; 1.39, 2.13.4 and 3.25; 1.39, 2.13.3 and 3.26; 1.39, 2.13.4 and 3.26; 1.39, 2.13.3 and 3.27; 1.39, 2.13.4 and 3.27; 1.39, 2.13.3 and 3.28; 1.39, 2.13.4 and 3.28; 1.39, 2.13.3 and 3.29; 1.39, 2.13.4 and 3.29; 1.39, 2.13.3 and 3.30; 1.39, 2.13.4 and 3.30; 1.39, 2.13.3 and 3.31; 1.39, 2.13.4 and 3.31; 1.39, 2.13.3 and 3.33; 1.39, 2.13.4 and 3.33; 1.39, 2.13.3 and 3.34; 1.39, 2.13.4 and 3.34; 1.39, 2.13.3 and 3.35; 1.39, 2.13.4 and 3.35; 1.39, 2.13.3 and 3.36; 1.39, 2.13.4 and 3.36; 1.39, 2.13.3 and 3.37; 1.39, 2.13.3 and 3.38; 1.39, 2.13.4 and 3.37; 1.39, 2.13.4 and 3.38; 1.40, 2.1 and 3.1; 1.40, 2.2 and 3.1; 1.40, 2.3 and 3.1; 1.40, 2.1 and 3.3; 1.40, 2.2 and 3.3; 1.40, 2.3 and 3.3; 1.40, 2.1 and 3.4; 1.40, 2.2 and 3.4; 1.40, 2.3 and 3.4; 1.40, 2.1 and 3.8; 1.40, 2.2 and 3.8; 1.40, 2.3 and 3.8; 1.40, 2.1 and 3.9; 1.40, 2.2 and 3.9; 1.40, 2.3 and 3.9; 1.40, 2.1 and 3.16; 1.40, 2.2 and 3.16; 1.40, 2.3 and 3.16; 1.40, 2.1 and 3.17; 1.40, 2.2 and 3.17; 1.40, 2.3 and 3.17; 1.40, 2.1 and 3.20; 1.40, 2.2 and 3.20; 1.40, 2.3 and 3.20; 1.40, 2.1 and 3.24; 1.40, 2.2 and 3.24; 1.40, 2.3 and 3.24; 1.40, 2.1 and 3.25; 1.40, 2.2 and 3.25; 1.40, 2.3 and 3.25; 1.40, 2.1 and 3.26; 1.40, 2.2 and 3.26; 1.40, 2.3 and 3.26; 1.40, 2.1 and 3.27; 1.40, 2.2 and 3.27; 1.40, 2.3 and 3.27; 1.40, 2.1 and 3.28; 1.40, 2.2 and 3.28; 1.40, 2.3 and 3.28; 1.40, 2.1 and 3.29; 1.40, 2.2 and 3.29; 1.40, 2.3 and 3.29; 1.40, 2.1 and 3.30; 1.40, 2.2 and 3.30; 1.40, 2.3 and 3.30; 1.40, 2.1 and 3.31; 1.40, 2.2 and 3.31; 1.40, 2.3 and 3.31; 1.40, 2.1 and 3.33; 1.40, 2.2 and 3.33; 1.40, 2.3 and 3.33; 1.40, 2.1 and 3.34; 1.40, 2.2 and 3.34; 1.40, 2.3 and 3.34; 1.40, 2.1 and 3.35; 1.40, 2.2 and 3.35; 1.40, 2.3 and 3.35; 1.40, 2.1 and 3.36; 1.40, 2.2 and 3.36; 1.40, 2.3 and 3.36; 1.40, 2.1 and 3.37; 1.40, 2.1 and 3.38; 1.40, 2.2 and 3.37; 1.40, 2.2 and 3.38; 1.40, 2.3 and 3.37; 1.40, 2.3 and 3.38; 1.40, 2.4 and 3.1; 1.40, 2.5 and 3.1; 1.40, 2.7 and 3.1; 1.40, 2.4 and 3.3; 1.40, 2.5 and 3.3; 1.40, 2.7 and 3.3; 1.40, 2.4 and 3.4; 1.40, 2.5 and 3.4; 1.40, 2.7 and 3.4; 1.40, 2.4 and 3.8; 1.40, 2.5 and 3.8; 1.40, 2.7 and 3.8; 1.40, 2.4 and 3.9; 1.40, 2.5 and 3.9; 1.40, 2.7 and 3.9; 1.40, 2.4 and 3.16; 1.40, 2.5 and 3.16; 1.40, 2.7 and 3.16; 1.40, 2.4 and 3.17; 1.40, 2.5 and 3.17; 1.40, 2.7 and 3.17; 1.40, 2.4 and 3.20; 1.40, 2.5 and 3.20; 1.40, 2.7 and 3.20; 1.40, 2.4 and 3.24; 1.40, 2.5 and 3.24; 1.40, 2.7 and 3.24; 1.40, 2.4 and 3.25; 1.40, 2.5 and 3.25; 1.40, 2.7 and 3.25; 1.40, 2.4 and 3.26; 1.40, 2.5 and 3.26; 1.40, 2.7 and 3.26; 1.40, 2.4 and 3.27; 1.40, 2.5 and 3.27; 1.40, 2.7 and 3.27; 1.40, 2.4 and 3.28; 1.40, 2.5 and 3.28; 1.40, 2.7 and 3.28; 1.40, 2.4 and 3.29; 1.40, 2.5 and 3.29; 1.40, 2.7 and 3.29; 1.40, 2.4 and 3.30; 1.40, 2.5 and 3.30; 1.40, 2.7 and 3.30; 1.40, 2.4 and 3.31; 1.40, 2.5 and 3.31; 1.40, 2.7 and 3.31; 1.40, 2.4 and 3.33; 1.40, 2.5 and 3.33; 1.40, 2.7 and 3.33; 1.40, 2.4 and 3.34; 1.40, 2.5 and 3.34; 1.40, 2.7 and 3.34; 1.40, 2.4 and 3.35; 1.40, 2.5 and 3.35; 1.40, 2.7 and 3.35; 1.40, 2.4 and 3.36; 1.40, 2.5 and 3.36; 1.40, 2.7 and 3.36; 1.40, 2.4 and 3.37; 1.40, 2.4 and 3.38; 1.40, 2.5 and 3.37; 1.40, 2.5 and 3.38; 1.40, 2.7 and 3.37; 1.40, 2.7 and 3.38; 1.40, 2.9.1 and 3.1; 1.40, 2.9.2 and 3.1; 1.40, 2.9.1 and 3.3; 1.40, 2.9.2 and 3.3; 1.40, 2.9.1 and 3.4; 1.40, 2.9.2 and 3.4; 1.40, 2.9.1 and 3.8; 1.40, 2.9.2 and 3.8; 1.40, 2.9.1 and 3.9; 1.40, 2.9.2 and 3.9; 1.40, 2.9.1 and 3.16; 1.40, 2.9.2 and 3.16; 1.40, 2.9.1 and 3.17; 1.40, 2.9.2 and 3.17; 1.40, 2.9.1 and 3.20; 1.40, 2.9.2 and 3.20; 1.40, 2.9.1 and 3.24; 1.40, 2.9.2 and 3.24; 1.40, 2.9.1 and 3.25; 1.40, 2.9.2 and 3.25; 1.40, 2.9.1 and 3.26; 1.40, 2.9.2 and 3.26; 1.40, 2.9.1 and 3.27; 1.40, 2.9.2 and 3.27; 1.40, 2.9.1 and 3.28; 1.40, 2.9.2 and 3.28; 1.40, 2.9.1 and 3.29; 1.40, 2.9.2 and 3.29; 1.40, 2.9.1 and 3.30; 1.40, 2.9.2 and 3.30; 1.40, 2.9.1 and 3.31; 1.40, 2.9.2 and 3.31; 1.40, 2.9.1 and 3.33; 1.40, 2.9.2 and 3.33; 1.40, 2.9.1 and 3.34; 1.40, 2.9.2 and 3.34; 1.40, 2.9.1 and 3.35; 1.40, 2.9.2 and 3.35; 1.40, 2.9.1 and 3.36; 1.40, 2.9.2 and 3.36; 1.40, 2.9.1 and 3.37; 1.40, 2.9.1 and 3.38; 1.40, 2.9.2 and 3.37; 1.40, 2.9.2 and 3.38; 1.40, 2.10.1 and 3.1; 1.40, 2.10.2 and 3.1; 1.40, 2.10.1 and 3.3; 1.40, 2.10.2 and 3.3; 1.40, 2.10.1 and 3.4; 1.40, 2.10.2 and 3.4; 1.40, 2.10.1 and 3.8; 1.40, 2.10.2 and 3.8; 1.40, 2.10.1 and 3.9; 1.40, 2.10.2 and 3.9; 1.40, 2.10.1 and 3.16; 1.40, 2.10.2 and 3.16; 1.40, 2.10.1 and 3.17; 1.40, 2.10.2 and 3.17; 1.40, 2.10.1 and 3.20; 1.40, 2.10.2 and 3.20; 1.40, 2.10.1 and 3.24; 1.40, 2.10.2 and 3.24; 1.40, 2.10.1 and 3.25; 1.40, 2.10.2 and 3.25; 1.40, 2.10.1 and 3.26; 1.40, 2.10.2 and 3.26; 1.40, 2.10.1 and 3.27; 1.40, 2.10.2 and 3.27; 1.40, 2.10.1 and 3.28; 1.40, 2.10.2 and 3.28; 1.40, 2.10.1 and 3.29; 1.40, 2.10.2 and 3.29; 1.40, 2.10.1 and 3.30; 1.40, 2.10.2 and 3.30; 1.40, 2.10.1 and 3.31; 1.40, 2.10.2 and 3.31; 1.40, 2.10.1 and 3.33; 1.40, 2.10.2 and 3.33; 1.40, 2.10.1 and 3.34; 1.40, 2.10.2 and 3.34; 1.40, 2.10.1 and 3.35; 1.40, 2.10.2 and 3.35; 1.40, 2.10.1 and 3.36; 1.40, 2.10.2 and 3.36; 1.40, 2.10.1 and 3.37; 1.40, 2.10.1 and 3.38; 1.40, 2.10.2 and 3.37; 1.40, 2.10.2 and 3.38; 1.40, 2.11.5 and 3.1; 1.40, 2.11.5 and 3.3; 1.40, 2.11.5 and 3.4; 1.40, 2.11.5 and 3.8; 1.40, 2.11.5 and 3.9; 1.40, 2.11.5 and 3.16; 1.40, 2.11.5 and 3.17; 1.40, 2.11.5 and 3.20; 1.40, 2.11.5 and 3.24; 1.40, 2.11.5 and 3.25; 1.40, 2.11.5 and 3.26; 1.40, 2.11.5 and 3.27; 1.40, 2.11.5 and 3.28; 1.40, 2.11.5 and 3.29; 1.40, 2.11.5 and 3.30; 1.40, 2.11.5 and 3.31; 1.40, 2.11.5 and 3.33; 1.40, 2.11.5 and 3.34; 1.40, 2.11.5 and 3.35; 1.40, 2.11.5 and 3.36; 1.40, 2.11.5 and 3.37; 1.40, 2.11.5 and 3.38; 1.40, 2.11.6 and 3.1; 1.40, 2.12.1 and 3.1; 1.40, 2.12.2 and 3.1; 1.40, 2.11.6 and 3.3; 1.40, 2.12.1 and 3.3; 1.40, 2.12.2 and 3.3; 1.40, 2.11.6 and 3.4; 1.40, 2.12.1 and 3.4; 1.40, 2.12.2 and 3.4; 1.40, 2.11.6 and 3.8; 1.40, 2.12.1 and 3.8; 1.40, 2.12.2 and 3.8; 1.40, 2.11.6 and 3.9; 1.40, 2.12.1 and 3.9; 1.40, 2.12.2 and 3.9; 1.40, 2.11.6 and 3.16; 1.40, 2.12.1 and 3.16; 1.40, 2.12.2 and 3.16; 1.40, 2.11.6 and 3.17; 1.40, 2.12.1 and 3.17; 1.40, 2.12.2 and 3.17; 1.40, 2.11.6 and 3.20; 1.40, 2.12.1 and 3.20; 1.40, 2.12.2 and 3.20; 1.40, 2.11.6 and 3.24; 1.40, 2.12.1 and 3.24; 1.40, 2.12.2 and 3.24; 1.40, 2.11.6 and 3.25; 1.40, 2.12.1 and 3.25; 1.40, 2.12.2 and 3.25; 1.40, 2.11.6 and 3.26; 1.40, 2.12.1 and 3.26; 1.40, 2.12.2 and 3.26; 1.40, 2.11.6 and 3.27; 1.40, 2.12.1 and 3.27; 1.40, 2.12.2 and 3.27; 1.40, 2.11.6 and 3.28; 1.40, 2.12.1 and 3.28; 1.40, 2.12.2 and 3.28; 1.40, 2.11.6 and 3.29; 1.40, 2.12.1 and 3.29; 1.40, 2.12.2 and 3.29; 1.40, 2.11.6 and 3.30; 1.40, 2.12.1 and 3.30; 1.40, 2.12.2 and 3.30; 1.40, 2.11.6 and 3.31; 1.40, 2.12.1 and 3.31; 1.40, 2.12.2 and 3.31; 1.40, 2.11.6 and 3.33; 1.40, 2.12.1 and 3.33; 1.40, 2.12.2 and 3.33; 1.40, 2.11.6 and 3.34; 1.40, 2.12.1 and 3.34; 1.40, 2.12.2 and 3.34; 1.40, 2.11.6 and 3.35; 1.40, 2.12.1 and 3.35; 1.40, 2.12.2 and 3.35; 1.40, 2.11.6 and 3.36; 1.40, 2.12.1 and 3.36; 1.40, 2.12.2 and 3.36; 1.40, 2.11.6 and 3.37; 1.40, 2.11.6 and 3.38; 1.40, 2.12.1 and 3.37; 1.40, 2.12.1 and 3.38; 1.40, 2.12.2 and 3.37; 1.40, 2.12.2 and 3.38; 1.40, 2.13.3 and 3.1; 1.40, 2.13.4 and 3.1; 1.40, 2.13.3 and 3.3; 1.40, 2.13.4 and 3.3; 1.40, 2.13.3 and 3.4; 1.40, 2.13.4 and 3.4; 1.40, 2.13.3 and 3.8; 1.40, 2.13.4 and 3.8; 1.40, 2.13.3 and 3.9; 1.40, 2.13.4 and 3.9; 1.40, 2.13.3 and 3.16; 1.40, 2.13.4 and 3.16; 1.40, 2.13.3 and 3.17; 1.40, 2.13.4 and 3.17; 1.40, 2.13.3 and 3.20; 1.40, 2.13.4 and 3.20; 1.40, 2.13.3 and 3.24; 1.40, 2.13.4 and 3.24; 1.40, 2.13.3 and 3.25; 1.40, 2.13.4 and 3.25; 1.40, 2.13.3 and 3.26; 1.40, 2.13.4 and 3.26; 1.40, 2.13.3 and 3.27; 1.40, 2.13.4 and 3.27; 1.40, 2.13.3 and 3.28; 1.40, 2.13.4 and 3.28; 1.40, 2.13.3 and 3.29; 1.40, 2.13.4 and 3.29; 1.40, 2.13.3 and 3.30; 1.40, 2.13.4 and 3.30; 1.40, 2.13.3 and 3.31; 1.40, 2.13.4 and 3.31; 1.40, 2.13.3 and 3.33; 1.40, 2.13.4 and 3.33; 1.40, 2.13.3 and 3.34; 1.40, 2.13.4 and 3.34; 1.40, 2.13.3 and 3.35; 1.40, 2.13.4 and 3.35; 1.40, 2.13.3 and 3.36; 1.40, 2.13.4 and 3.36; 1.40, 2.13.3 and 3.37; 1.40, 2.13.3 and 3.38; 1.40, 2.13.4 and 3.37; 1.40, 2.13.4 and 3.38; 1.41, 2.1 and 3.1; 1.41, 2.2 and 3.1; 1.41, 2.3 and 3.1; 1.41, 2.1 and 3.3; 1.41, 2.2 and 3.3; 1.41, 2.3 and 3.3; 1.41, 2.1 and 3.4; 1.41, 2.2 and 3.4; 1.41, 2.3 and 3.4; 1.41, 2.1 and 3.8; 1.41, 2.2 and 3.8; 1.41, 2.3 and 3.8; 1.41, 2.1 and 3.9; 1.41, 2.2 and 3.9; 1.41, 2.3 and 3.9; 1.41, 2.1 and 3.16; 1.41, 2.2 and 3.16; 1.41, 2.3 and 3.16; 1.41, 2.1 and 3.17; 1.41, 2.2 and 3.17; 1.41, 2.3 and 3.17; 1.41, 2.1 and 3.20; 1.41, 2.2 and 3.20; 1.41, 2.3 and 3.20; 1.41, 2.1 and 3.24; 1.41, 2.2 and 3.24; 1.41, 2.3 and 3.24; 1.41, 2.1 and 3.25; 1.41, 2.2 and 3.25; 1.41, 2.3 and 3.25; 1.41, 2.1 and 3.26; 1.41, 2.2 and 3.26; 1.41, 2.3 and 3.26; 1.41, 2.1 and 3.27; 1.41, 2.2 and 3.27; 1.41, 2.3 and 3.27; 1.41, 2.1 and 3.28; 1.41, 2.2 and 3.28; 1.41, 2.3 and 3.28; 1.41, 2.1 and 3.29; 1.41, 2.2 and 3.29; 1.41, 2.3 and 3.29; 1.41, 2.1 and 3.30; 1.41, 2.2 and 3.30; 1.41, 2.3 and 3.30; 1.41, 2.1 and 3.31; 1.41, 2.2 and 3.31; 1.41, 2.3 and 3.31; 1.41, 2.1 and 3.33; 1.41, 2.2 and 3.33; 1.41, 2.3 and 3.33; 1.41, 2.1 and 3.34; 1.41, 2.2 and 3.34; 1.41, 2.3 and 3.34; 1.41, 2.1 and 3.35; 1.41, 2.2 and 3.35; 1.41, 2.3 and 3.35; 1.41, 2.1 and 3.36; 1.41, 2.2 and 3.36; 1.41, 2.3 and 3.36; 1.41, 2.1 and 3.37; 1.41, 2.1 and 3.38; 1.41, 2.2 and 3.37; 1.41, 2.2 and 3.38; 1.41, 2.3 and 3.37; 1.41, 2.3 and 3.38; 1.41, 2.4 and 3.1; 1.41, 2.5 and 3.1; 1.41, 2.7 and 3.1; 1.41, 2.4 and 3.3; 1.41, 2.5 and 3.3; 1.41, 2.7 and 3.3; 1.41, 2.4 and 3.4; 1.41, 2.5 and 3.4; 1.41, 2.7 and 3.4; 1.41, 2.4 and 3.8; 1.41, 2.5 and 3.8; 1.41, 2.7 and 3.8; 1.41, 2.4 and 3.9; 1.41, 2.5 and 3.9; 1.41, 2.7 and 3.9; 1.41, 2.4 and 3.16; 1.41, 2.5 and 3.16; 1.41, 2.7 and 3.16; 1.41, 2.4 and 3.17; 1.41, 2.5 and 3.17; 1.41, 2.7 and 3.17; 1.41, 2.4 and 3.20; 1.41, 2.5 and 3.20; 1.41, 2.7 and 3.20; 1.41, 2.4 and 3.24; 1.41, 2.5 and 3.24; 1.41, 2.7 and 3.24; 1.41, 2.4 and 3.25; 1.41, 2.5 and 3.25; 1.41, 2.7 and 3.25; 1.41, 2.4 and 3.26; 1.41, 2.5 and 3.26; 1.41, 2.7 and 3.26; 1.41, 2.4 and 3.27; 1.41, 2.5 and 3.27; 1.41, 2.7 and 3.27; 1.41, 2.4 and 3.28; 1.41, 2.5 and 3.28; 1.41, 2.7 and 3.28; 1.41, 2.4 and 3.29; 1.41, 2.5 and 3.29; 1.41, 2.7 and 3.29; 1.41, 2.4 and 3.30; 1.41, 2.5 and 3.30; 1.41, 2.7 and 3.30; 1.41, 2.4 and 3.31; 1.41, 2.5 and 3.31; 1.41, 2.7 and 3.31; 1.41, 2.4 and 3.33; 1.41, 2.5 and 3.33; 1.41, 2.7 and 3.33; 1.41, 2.4 and 3.34; 1.41, 2.5 and 3.34; 1.41, 2.7 and 3.34; 1.41, 2.4 and 3.35; 1.41, 2.5 and 3.35; 1.41, 2.7 and 3.35; 1.41, 2.4 and 3.36; 1.41, 2.5 and 3.36; 1.41, 2.7 and 3.36; 1.41, 2.4 and 3.37; 1.41, 2.4 and 3.38; 1.41, 2.5 and 3.37; 1.41, 2.5 and 3.38; 1.41, 2.7 and 3.37; 1.41, 2.7 and 3.38; 1.41, 2.9.1 and 3.1; 1.41, 2.9.2 and 3.1; 1.41, 2.9.1 and 3.3; 1.41, 2.9.2 and 3.3; 1.41, 2.9.1 and 3.4; 1.41, 2.9.2 and 3.4; 1.41, 2.9.1 and 3.8; 1.41, 2.9.2 and 3.8; 1.41, 2.9.1 and 3.9; 1.41, 2.9.2 and 3.9; 1.41, 2.9.1 and 3.16; 1.41, 2.9.2 and 3.16; 1.41, 2.9.1 and 3.17; 1.41, 2.9.2 and 3.17; 1.41, 2.9.1 and 3.20; 1.41, 2.9.2 and 3.20; 1.41, 2.9.1 and 3.24; 1.41, 2.9.2 and 3.24; 1.41, 2.9.1 and 3.25; 1.41, 2.9.2 and 3.25; 1.41, 2.9.1 and 3.26; 1.41, 2.9.2 and 3.26; 1.41, 2.9.1 and 3.27; 1.41, 2.9.2 and 3.27; 1.41, 2.9.1 and 3.28; 1.41, 2.9.2 and 3.28; 1.41, 2.9.1 and 3.29; 1.41, 2.9.2 and 3.29; 1.41, 2.9.1 and 3.30; 1.41, 2.9.2 and 3.30; 1.41, 2.9.1 and 3.31; 1.41, 2.9.2 and 3.31; 1.41, 2.9.1 and 3.33; 1.41, 2.9.2 and 3.33; 1.41, 2.9.1 and 3.34; 1.41, 2.9.2 and 3.34; 1.41, 2.9.1 and 3.35; 1.41, 2.9.2 and 3.35; 1.41, 2.9.1 and 3.36; 1.41, 2.9.2 and 3.36; 1.41, 2.9.1 and 3.37; 1.41, 2.9.1 and 3.38; 1.41, 2.9.2 and 3.37; 1.41, 2.9.2 and 3.38; 1.41, 2.10.1 and 3.1; 1.41, 2.10.2 and 3.1; 1.41, 2.10.1 and 3.3; 1.41, 2.10.2 and 3.3; 1.41, 2.10.1 and 3.4; 1.41, 2.10.2 and 3.4; 1.41, 2.10.1 and 3.8; 1.41, 2.10.2 and 3.8; 1.41, 2.10.1 and 3.9; 1.41, 2.10.2 and 3.9; 1.41, 2.10.1 and 3.16; 1.41, 2.10.2 and 3.16; 1.41, 2.10.1 and 3.17; 1.41, 2.10.2 and 3.17; 1.41, 2.10.1 and 3.20; 1.41, 2.10.2 and 3.20; 1.41, 2.10.1 and 3.24; 1.41, 2.10.2 and 3.24; 1.41, 2.10.1 and 3.25; 1.41, 2.10.2 and 3.25; 1.41, 2.10.1 and 3.26; 1.41, 2.10.2 and 3.26; 1.41, 2.10.1 and 3.27; 1.41, 2.10.2 and 3.27; 1.41, 2.10.1 and 3.28; 1.41, 2.10.2 and 3.28; 1.41, 2.10.1 and 3.29; 1.41, 2.10.2 and 3.29; 1.41, 2.10.1 and 3.30; 1.41, 2.10.2 and 3.30; 1.41, 2.10.1 and 3.31; 1.41, 2.10.2 and 3.31; 1.41, 2.10.1 and 3.33; 1.41, 2.10.2 and 3.33; 1.41, 2.10.1 and 3.34; 1.41, 2.10.2 and 3.34; 1.41, 2.10.1 and 3.35; 1.41, 2.10.2 and 3.35; 1.41, 2.10.1 and 3.36; 1.41, 2.10.2 and 3.36; 1.41, 2.10.1 and 3.37; 1.41, 2.10.1 and 3.38; 1.41, 2.10.2 and 3.37; 1.41, 2.10.2 and 3.38; 1.41, 2.11.5 and 3.1; 1.41, 2.11.5 and 3.3; 1.41, 2.11.5 and 3.4; 1.41, 2.11.5 and 3.8; 1.41, 2.11.5 and 3.9; 1.41, 2.11.5 and 3.16; 1.41, 2.11.5 and 3.17; 1.41, 2.11.5 and 3.20; 1.41, 2.11.5 and 3.24; 1.41, 2.11.5 and 3.25; 1.41, 2.11.5 and 3.26; 1.41, 2.11.5 and 3.27; 1.41, 2.11.5 and 3.28; 1.41, 2.11.5 and 3.29; 1.41, 2.11.5 and 3.30; 1.41, 2.11.5 and 3.31; 1.41, 2.11.5 and 3.33; 1.41, 2.11.5 and 3.34; 1.41, 2.11.5 and 3.35; 1.41, 2.11.5 and 3.36; 1.41, 2.11.5 and 3.37; 1.41, 2.11.5 and 3.38; 1.41, 2.11.6 and 3.1; 1.41, 2.12.1 and 3.1; 1.41, 2.12.2 and 3.1; 1.41, 2.11.6 and 3.3; 1.41, 2.12.1 and 3.3; 1.41, 2.12.2 and 3.3; 1.41, 2.11.6 and 3.4; 1.41, 2.12.1 and 3.4; 1.41, 2.12.2 and 3.4; 1.41, 2.11.6 and 3.8; 1.41, 2.12.1 and 3.8; 1.41, 2.12.2 and 3.8; 1.41, 2.11.6 and 3.9; 1.41, 2.12.1 and 3.9; 1.41, 2.12.2 and 3.9; 1.41, 2.11.6 and 3.16; 1.41, 2.12.1 and 3.16; 1.41, 2.12.2 and 3.16; 1.41, 2.11.6 and 3.17; 1.41, 2.12.1 and 3.17; 1.41, 2.12.2 and 3.17; 1.41, 2.11.6 and 3.20; 1.41, 2.12.1 and 3.20; 1.41, 2.12.2 and 3.20; 1.41, 2.11.6 and 3.24; 1.41, 2.12.1 and 3.24; 1.41, 2.12.2 and 3.24; 1.41, 2.11.6 and 3.25; 1.41, 2.12.1 and 3.25; 1.41, 2.12.2 and 3.25; 1.41, 2.11.6 and 3.26; 1.41, 2.12.1 and 3.26; 1.41, 2.12.2 and 3.26; 1.41, 2.11.6 and 3.27; 1.41, 2.12.1 and 3.27; 1.41, 2.12.2 and 3.27; 1.41, 2.11.6 and 3.28; 1.41, 2.12.1 and 3.28; 1.41, 2.12.2 and 3.28; 1.41, 2.11.6 and 3.29; 1.41, 2.12.1 and 3.29; 1.41, 2.12.2 and 3.29; 1.41, 2.11.6 and 3.30; 1.41, 2.12.1 and 3.30; 1.41, 2.12.2 and 3.30; 1.41, 2.11.6 and 3.31; 1.41, 2.12.1 and 3.31; 1.41, 2.12.2 and 3.31; 1.41, 2.11.6 and 3.33; 1.41, 2.12.1 and 3.33; 1.41, 2.12.2 and 3.33; 1.41, 2.11.6 and 3.34; 1.41, 2.12.1 and 3.34; 1.41, 2.12.2 and 3.34; 1.41, 2.11.6 and 3.35; 1.41, 2.12.1 and 3.35; 1.41, 2.12.2 and 3.35; 1.41, 2.11.6 and 3.36; 1.41, 2.12.1 and 3.36; 1.41, 2.12.2 and 3.36; 1.41, 2.11.6 and 3.37; 1.41, 2.11.6 and 3.38; 1.41, 2.12.1 and 3.37; 1.41, 2.12.1 and 3.38; 1.41, 2.12.2 and 3.37; 1.41, 2.12.2 and 3.38; 1.41, 2.13.3 and 3.1; 1.41, 2.13.4 and 3.1; 1.41, 2.13.3 and 3.3; 1.41, 2.13.4 and 3.3; 1.41, 2.13.3 and 3.4; 1.41, 2.13.4 and 3.4; 1.41, 2.13.3 and 3.8; 1.41, 2.13.4 and 3.8; 1.41, 2.13.3 and 3.9; 1.41, 2.13.4 and 3.9; 1.41, 2.13.3 and 3.16; 1.41, 2.13.4 and 3.16; 1.41, 2.13.3 and 3.17; 1.41, 2.13.4 and 3.17; 1.41, 2.13.3 and 3.20; 1.41, 2.13.4 and 3.20; 1.41, 2.13.3 and 3.24; 1.41, 2.13.4 and 3.24; 1.41, 2.13.3 and 3.25; 1.41, 2.13.4 and 3.25; 1.41, 2.13.3 and 3.26; 1.41, 2.13.4 and 3.26; 1.41, 2.13.3 and 3.27; 1.41, 2.13.4 and 3.27; 1.41, 2.13.3 and 3.28; 1.41, 2.13.4 and 3.28; 1.41, 2.13.3 and 3.29; 1.41, 2.13.4 and 3.29; 1.41, 2.13.3 and 3.30; 1.41, 2.13.4 and 3.30; 1.41, 2.13.3 and 3.31; 1.41, 2.13.4 and 3.31; 1.41, 2.13.3 and 3.33; 1.41, 2.13.4 and 3.33; 1.41, 2.13.3 and 3.34; 1.41, 2.13.4 and 3.34; 1.41, 2.13.3 and 3.35; 1.41, 2.13.4 and 3.35; 1.41, 2.13.3 and 3.36; 1.41, 2.13.4 and 3.36; 1.41, 2.13.3 and 3.37; 1.41, 2.13.3 and 3.38; 1.41, 2.13.4 and 3.37; 1.41, 2.13.4 and 3.38; 1.45, 2.1 and 3.1; 1.45, 2.2 and 3.1; 1.45, 2.3 and 3.1; 1.45, 2.1 and 3.3; 1.45, 2.2 and 3.3; 1.45, 2.3 and 3.3; 1.45, 2.1 and 3.4; 1.45, 2.2 and 3.4; 1.45, 2.3 and 3.4; 1.45, 2.1 and 3.8; 1.45, 2.2 and 3.8; 1.45, 2.3 and 3.8; 1.45, 2.1 and 3.9; 1.45, 2.2 and 3.9; 1.45, 2.3 and 3.9; 1.45, 2.1 and 3.16; 1.45, 2.2 and 3.16; 1.45, 2.3 and 3.16; 1.45, 2.1 and 3.17; 1.45, 2.2 and 3.17; 1.45, 2.3 and 3.17; 1.45, 2.1 and 3.20; 1.45, 2.2 and 3.20; 1.45, 2.3 and 3.20; 1.45, 2.1 and 3.24; 1.45, 2.2 and 3.24; 1.45, 2.3 and 3.24; 1.45, 2.1 and 3.25; 1.45, 2.2 and 3.25; 1.45, 2.3 and 3.25; 1.45, 2.1 and 3.26; 1.45, 2.2 and 3.26; 1.45, 2.3 and 3.26; 1.45, 2.1 and 3.27; 1.45, 2.2 and 3.27; 1.45, 2.3 and 3.27; 1.45, 2.1 and 3.28; 1.45, 2.2 and 3.28; 1.45, 2.3 and 3.28; 1.45, 2.1 and 3.29; 1.45, 2.2 and 3.29; 1.45, 2.3 and 3.29; 1.45, 2.1 and 3.30; 1.45, 2.2 and 3.30; 1.45, 2.3 and 3.30; 1.45, 2.1 and 3.31; 1.45, 2.2 and 3.31; 1.45, 2.3 and 3.31; 1.45, 2.1 and 3.33; 1.45, 2.2 and 3.33; 1.45, 2.3 and 3.33; 1.45, 2.1 and 3.34; 1.45, 2.2 and 3.34; 1.45, 2.3 and 3.34; 1.45, 2.1 and 3.35; 1.45, 2.2 and 3.35; 1.45, 2.3 and 3.35; 1.45, 2.1 and 3.36; 1.45, 2.2 and 3.36; 1.45, 2.3 and 3.36; 1.45, 2.1 and 3.37; 1.45, 2.1 and 3.38; 1.45, 2.2 and 3.37; 1.45, 2.2 and 3.38; 1.45, 2.3 and 3.37; 1.45, 2.3 and 3.38; 1.45, 2.4 and 3.1; 1.45, 2.5 and 3.1; 1.45, 2.7 and 3.1; 1.45, 2.4 and 3.3; 1.45, 2.5 and 3.3; 1.45, 2.7 and 3.3; 1.45, 2.4 and 3.4; 1.45, 2.5 and 3.4; 1.45, 2.7 and 3.4; 1.45, 2.4 and 3.8; 1.45, 2.5 and 3.8; 1.45, 2.7 and 3.8; 1.45, 2.4 and 3.9; 1.45, 2.5 and 3.9; 1.45, 2.7 and 3.9; 1.45, 2.4 and 3.16; 1.45, 2.5 and 3.16; 1.45, 2.7 and 3.16; 1.45, 2.4 and 3.17; 1.45, 2.5 and 3.17; 1.45, 2.7 and 3.17; 1.45, 2.4 and 3.20; 1.45, 2.5 and 3.20; 1.45, 2.7 and 3.20; 1.45, 2.4 and 3.24; 1.45, 2.5 and 3.24; 1.45, 2.7 and 3.24; 1.45, 2.4 and 3.25; 1.45, 2.5 and 3.25; 1.45, 2.7 and 3.25; 1.45, 2.4 and 3.26; 1.45, 2.5 and 3.26; 1.45, 2.7 and 3.26; 1.45, 2.4 and 3.27; 1.45, 2.5 and 3.27; 1.45, 2.7 and 3.27; 1.45, 2.4 and 3.28; 1.45, 2.5 and 3.28; 1.45, 2.7 and 3.28; 1.45, 2.4 and 3.29; 1.45, 2.5 and 3.29; 1.45, 2.7 and 3.29; 1.45, 2.4 and 3.30; 1.45, 2.5 and 3.30; 1.45, 2.7 and 3.30; 1.45, 2.4 and 3.31; 1.45, 2.5 and 3.31; 1.45, 2.7 and 3.31; 1.45, 2.4 and 3.33; 1.45, 2.5 and 3.33; 1.45, 2.7 and 3.33; 1.45, 2.4 and 3.34; 1.45, 2.5 and 3.34; 1.45, 2.7 and 3.34; 1.45, 2.4 and 3.35; 1.45, 2.5 and 3.35; 1.45, 2.7 and 3.35; 1.45, 2.4 and 3.36; 1.45, 2.5 and 3.36; 1.45, 2.7 and 3.36; 1.45, 2.4 and 3.37; 1.45, 2.4 and 3.38; 1.45, 2.5 and 3.37; 1.45, 2.5 and 3.38; 1.45, 2.7 and 3.37; 1.45, 2.7 and 3.38; 1.45, 2.9.1 and 3.1; 1.45, 2.9.2 and 3.1; 1.45, 2.9.1 and 3.3; 1.45, 2.9.2 and 3.3; 1.45, 2.9.1 and 3.4; 1.45, 2.9.2 and 3.4; 1.45, 2.9.1 and 3.8; 1.45, 2.9.2 and 3.8; 1.45, 2.9.1 and 3.9; 1.45, 2.9.2 and 3.9; 1.45, 2.9.1 and 3.16; 1.45, 2.9.2 and 3.16; 1.45, 2.9.1 and 3.17; 1.45, 2.9.2 and 3.17; 1.45, 2.9.1 and 3.20; 1.45, 2.9.2 and 3.20; 1.45, 2.9.1 and 3.24; 1.45, 2.9.2 and 3.24; 1.45, 2.9.1 and 3.25; 1.45, 2.9.2 and 3.25; 1.45, 2.9.1 and 3.26; 1.45, 2.9.2 and 3.26; 1.45, 2.9.1 and 3.27; 1.45, 2.9.2 and 3.27; 1.45, 2.9.1 and 3.28; 1.45, 2.9.2 and 3.28; 1.45, 2.9.1 and 3.29; 1.45, 2.9.2 and 3.29; 1.45, 2.9.1 and 3.30; 1.45, 2.9.2 and 3.30; 1.45, 2.9.1 and 3.31; 1.45, 2.9.2 and 3.31; 1.45, 2.9.1 and 3.33; 1.45, 2.9.2 and 3.33; 1.45, 2.9.1 and 3.34; 1.45, 2.9.2 and 3.34; 1.45, 2.9.1 and 3.35; 1.45, 2.9.2 and 3.35; 1.45, 2.9.1 and 3.36; 1.45, 2.9.2 and 3.36; 1.45, 2.9.1 and 3.37; 1.45, 2.9.1 and 3.38; 1.45, 2.9.2 and 3.37; 1.45, 2.9.2 and 3.38; 1.45, 2.10.1 and 3.1; 1.45, 2.10.2 and 3.1; 1.45, 2.10.1 and 3.3; 1.45, 2.10.2 and 3.3; 1.45, 2.10.1 and 3.4; 1.45, 2.10.2 and 3.4; 1.45, 2.10.1 and 3.8; 1.45, 2.10.2 and 3.8; 1.45, 2.10.1 and 3.9; 1.45, 2.10.2 and 3.9; 1.45, 2.10.1 and 3.16; 1.45, 2.10.2 and 3.16; 1.45, 2.10.1 and 3.17; 1.45, 2.10.2 and 3.17; 1.45, 2.10.1 and 3.20; 1.45, 2.10.2 and 3.20; 1.45, 2.10.1 and 3.24; 1.45, 2.10.2 and 3.24; 1.45, 2.10.1 and 3.25; 1.45, 2.10.2 and 3.25; 1.45, 2.10.1 and 3.26; 1.45, 2.10.2 and 3.26; 1.45, 2.10.1 and 3.27; 1.45, 2.10.2 and 3.27; 1.45, 2.10.1 and 3.28; 1.45, 2.10.2 and 3.28; 1.45, 2.10.1 and 3.29; 1.45, 2.10.2 and 3.29; 1.45, 2.10.1 and 3.30; 1.45, 2.10.2 and 3.30; 1.45, 2.10.1 and 3.31; 1.45, 2.10.2 and 3.31; 1.45, 2.10.1 and 3.33; 1.45, 2.10.2 and 3.33; 1.45, 2.10.1 and 3.34; 1.45, 2.10.2 and 3.34; 1.45, 2.10.1 and 3.35; 1.45, 2.10.2 and 3.35; 1.45, 2.10.1 and 3.36; 1.45, 2.10.2 and 3.36; 1.45, 2.10.1 and 3.37; 1.45, 2.10.1 and 3.38; 1.45, 2.10.2 and 3.37; 1.45, 2.10.2 and 3.38; 1.45, 2.11.5 and 3.1; 1.45, 2.11.5 and 3.3; 1.45, 2.11.5 and 3.4; 1.45, 2.11.5 and 3.8; 1.45, 2.11.5 and 3.9; 1.45, 2.11.5 and 3.16; 1.45, 2.11.5 and 3.17; 1.45, 2.11.5 and 3.20; 1.45, 2.11.5 and 3.24; 1.45, 2.11.5 and 3.25; 1.45, 2.11.5 and 3.26; 1.45, 2.11.5 and 3.27; 1.45, 2.11.5 and 3.28; 1.45, 2.11.5 and 3.29; 1.45, 2.11.5 and 3.30; 1.45, 2.11.5 and 3.31; 1.45, 2.11.5 and 3.33; 1.45, 2.11.5 and 3.34; 1.45, 2.11.5 and 3.35; 1.45, 2.11.5 and 3.36; 1.45, 2.11.5 and 3.37; 1.45, 2.11.5 and 3.38; 1.45, 2.11.6 and 3.1; 1.45, 2.12.1 and 3.1; 1.45, 2.12.2 and 3.1; 1.45, 2.11.6 and 3.3; 1.45, 2.12.1 and 3.3; 1.45, 2.12.2 and 3.3; 1.45, 2.11.6 and 3.4; 1.45, 2.12.1 and 3.4; 1.45, 2.12.2 and 3.4; 1.45, 2.11.6 and 3.8; 1.45, 2.12.1 and 3.8; 1.45, 2.12.2 and 3.8; 1.45, 2.11.6 and 3.9; 1.45, 2.12.1 and 3.9; 1.45, 2.12.2 and 3.9; 1.45, 2.11.6 and 3.16; 1.45, 2.12.1 and 3.16; 1.45, 2.12.2 and 3.16; 1.45, 2.11.6 and 3.17; 1.45, 2.12.1 and 3.17; 1.45, 2.12.2 and 3.17; 1.45, 2.11.6 and 3.20; 1.45, 2.12.1 and 3.20; 1.45, 2.12.2 and 3.20; 1.45, 2.11.6 and 3.24; 1.45, 2.12.1 and 3.24; 1.45, 2.12.2 and 3.24; 1.45, 2.11.6 and 3.25; 1.45, 2.12.1 and 3.25; 1.45, 2.12.2 and 3.25; 1.45, 2.11.6 and 3.26; 1.45, 2.12.1 and 3.26; 1.45, 2.12.2 and 3.26; 1.45, 2.11.6 and 3.27; 1.45, 2.12.1 and 3.27; 1.45, 2.12.2 and 3.27; 1.45, 2.11.6 and 3.28; 1.45, 2.12.1 and 3.28; 1.45, 2.12.2 and 3.28; 1.45, 2.11.6 and 3.29; 1.45, 2.12.1 and 3.29; 1.45, 2.12.2 and 3.29; 1.45, 2.11.6 and 3.30; 1.45, 2.12.1 and 3.30; 1.45, 2.12.2 and 3.30; 1.45, 2.11.6 and 3.31; 1.45, 2.12.1 and 3.31; 1.45, 2.12.2 and 3.31; 1.45, 2.11.6 and 3.33; 1.45, 2.12.1 and 3.33; 1.45, 2.12.2 and 3.33; 1.45, 2.11.6 and 3.34; 1.45, 2.12.1 and 3.34; 1.45, 2.12.2 and 3.34; 1.45, 2.11.6 and 3.35; 1.45, 2.12.1 and 3.35; 1.45, 2.12.2 and 3.35; 1.45, 2.11.6 and 3.36; 1.45, 2.12.1 and 3.36; 1.45, 2.12.2 and 3.36; 1.45, 2.11.6 and 3.37; 1.45, 2.11.6 and 3.38; 1.45, 2.12.1 and 3.37; 1.45, 2.12.1 and 3.38; 1.45, 2.12.2 and 3.37; 1.45, 2.12.2 and 3.38; 1.45, 2.13.3 and 3.1; 1.45, 2.13.4 and 3.1; 1.45, 2.13.3 and 3.3; 1.45, 2.13.4 and 3.3; 1.45, 2.13.3 and 3.4; 1.45, 2.13.4 and 3.4; 1.45, 2.13.3 and 3.8; 1.45, 2.13.4 and 3.8; 1.45, 2.13.3 and 3.9; 1.45, 2.13.4 and 3.9; 1.45, 2.13.3 and 3.16; 1.45, 2.13.4 and 3.16; 1.45, 2.13.3 and 3.17; 1.45, 2.13.4 and 3.17; 1.45, 2.13.3 and 3.20; 1.45, 2.13.4 and 3.20; 1.45, 2.13.3 and 3.24; 1.45, 2.13.4 and 3.24; 1.45, 2.13.3 and 3.25; 1.45, 2.13.4 and 3.25; 1.45, 2.13.3 and 3.26; 1.45, 2.13.4 and 3.26; 1.45, 2.13.3 and 3.27; 1.45, 2.13.4 and 3.27; 1.45, 2.13.3 and 3.28; 1.45, 2.13.4 and 3.28; 1.45, 2.13.3 and 3.29; 1.45, 2.13.4 and 3.29; 1.45, 2.13.3 and 3.30; 1.45, 2.13.4 and 3.30; 1.45, 2.13.3 and 3.31; 1.45, 2.13.4 and 3.31; 1.45, 2.13.3 and 3.33; 1.45, 2.13.4 and 3.33; 1.45, 2.13.3 and 3.34; 1.45, 2.13.4 and 3.34; 1.45, 2.13.3 and 3.35; 1.45, 2.13.4 and 3.35; 1.45, 2.13.3 and 3.36; 1.45, 2.13.4 and 3.36; 1.45, 2.13.3 and 3.37; 1.45, 2.13.3 and 3.38; 1.45, 2.13.4 and 3.37 or 1.45, 2.13.4 and 3.38.

In a preferred embodiment the medicament combinations according to the invention contain as the betamimetic 1 one or more, preferably one compound selected from the group consisting of 1.8, 1.23, 1.30, 1.33, 1.34, and 1.45 more preferably selected from among 1.30, 1.33, and 1.34.

In a yet another preferred embodiment the medicament combinations according to the invention contain as the anticholinergic 2 one or more, preferably one compound selected from the group consisting of 2.1, 2.4, 2.5, 2.7, 2.9.1, 2.9.2, 2.12.1 and 2.12.2, more preferably selected from among 2.1, 2.5, 2.7, 2.9.1 and 2.9.2.

In a yet another preferred embodiment the medicament combinations according to the invention contain as the PDE IV inhibitor 3 one or more, preferably one compound selected from among 3.3, 3.8 and 3.35.

Definitions and Conventions Used

Unless otherwise stated or depicted, reference to a compound by chemical name or chemical structure is intended to cover, and does cover, all possible optical isomers, enantiomers, stereoisomers, diastereomers, and mixtures of any of the foregoing, including racemic mixtures (“racemates”).

Unless otherwise stated, the alkyl groups are straight-chained or branched alkyl groups having 1 to 4 carbon atoms. The following are mentioned by way of example: methyl, ethyl, propyl or butyl. In some cases the abbreviations Me, Et, Prop or Bu are used to denote the groups methyl, ethyl, propyl or butyl. Unless otherwise stated, the definitions propyl and butyl include all the possible isomeric forms of the groups in question. Thus, for example, propyl includes n-propyl and iso-propyl, butyl includes iso-butyl, sec.butyl and tert.-butyl, etc.

Unless otherwise stated, the cycloalkyl groups are alicyclic groups with 3 to 6 carbon atoms. They are the cyclopropyl, cyclobutyl, cyclopentyl and cyclohexyl groups. Cyclopropyl is particularly important within the scope of the present invention.

Unless otherwise stated, the alkylene groups are branched and unbranched double-bonded alkyl bridges with 1 to 4 carbon atoms. Examples include: methylene, ethylene, propylene or butylene.

Unless otherwise stated, the alkylene-halogen groups are branched and unbranched double-bonded alkyl bridges with 1 to 4 carbon atoms which are mono-, di- or trisubstituted, preferably disubstituted, by a halogen. Accordingly, unless otherwise stated, alkylene-OH-groups are branched and unbranched double-bonded alkyl bridges with 1 to 4 carbon atoms which are mono-, di- or trisubstituted, preferably monosubstituted, by a hydroxy.

Unless otherwise stated, the term alkyloxy groups denotes branched and unbranched alkyl groups with 1 to 4 carbon atoms which are linked via an oxygen atom. Examples include: methyloxy, ethyloxy, propyloxy or butyloxy. In some cases the abbreviations MeO, EtO, PropO or BuO may be used to denote the methyloxy, ethyloxy, propyloxy or butyloxy groups. Unless otherwise stated, the definitions propyloxy and butyloxy include all the possible isomeric forms of the groups in question. Thus, for example, propyloxy includes n-propyloxy and iso-propyloxy, butyloxy includes iso-butyloxy, sec.butyloxy and tert.-butyloxy, etc. In some cases the term alkoxy may be used instead of alkyloxy within the scope of the present invention. The groups methyloxy, ethyloxy, propyloxy or butyloxy may therefore also be referred to by the names methoxy, ethoxy, propoxy or butoxy.

Unless otherwise stated, the term alkylene-alkyloxy refers to branched and unbranched double-bonded alkyl bridges with 1 to 4 carbon atoms which are mono-, di- or trisubstituted, preferably monosubstituted, by an alkyloxy group.

Unless otherwise stated, the term —O—CO-alkyl groups refers to branched and unbranched alkyl groups with 1 to 4 carbon atoms which are linked by an ester group. The alkyl groups are attached directly to the carbonyl carbon of the ester group. The term —O—CO-alkyl-halogen should be understood analogously. The group —O—CO—CF₃ denotes trifluoroacetate.

Halogen within the scope of the present invention denotes fluorine, chlorine, bromine or iodine. Unless stated otherwise, fluorine and bromine are the preferred halogens. The group CO denotes a carbonyl group.

Therapeutic Use and Administration

Within the scope of the present invention by a pharmaceutical combination of components 1, 2 and 3 is meant the joint administration of the active substances in a single preparation or formulation or the separate administration of the active substances in separate formulations. If the active substances are administered in separate formulations, this separate administration may be done simultaneously or at different times, i.e. successively.

In one aspect the present invention relates to the above-mentioned medicament combinations which contain in addition to therapeutically effective amounts of 1, 2 and 3 a pharmaceutically acceptable carrier. In one aspect the present invention relates to the above-mentioned pharmaceutical compositions which do not contain contain a pharmaceutically acceptable carrier in addition to therapeutically effective amounts of 1, 2 and 3.

The present invention also relates to the use of therapeutically effective amounts of the active substances 1 for preparing a pharmaceutical composition also containing one or more, preferably one active substance 2 and one or more, preferably one active substance 3 for the treatment of inflammatory and obstructive respiratory complaints, for inhibiting premature labour in midwifery (tocolysis), for restoring sinus rhythm in the heart in atrioventricular block, for correcting bradycardic heart rhythm disorders (antiarrhythmic), for treating circulatory shock (vasodilatation and increasing the heart volume) as well as for the treatment of skin irritations and inflammation.

In a preferred aspect the present invention relates to the use of therapeutically effective amounts of the active substance 1 for preparing a pharmaceutical composition also containing one or more, preferably one, active substance 2 and one or more, preferably one active substance 3 for the treatment of respiratory complaints selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

Preferably the medicament combinations according to the invention are used as specified above for preparing a pharmaceutical composition for the treatment of obstructive pulmonary diseases selected from among bronchial asthma, paediatric asthma, severe asthma, acute asthma attacks, chronic bronchitis and COPD (chronic obstructive pulmonary disease), while it is particularly preferable according to the invention to use them for preparing a pharmaceutical composition for the treatment of bronchial asthma and COPD.

It is also preferable to use the medicament combinations according to the invention for preparing a pharmaceutical composition for the treatment of pulmonary emphysema which has its origins in COPD (chronic obstructive pulmonary disease) or □1-proteinase inhibitor deficiency.

It is also preferable to use the medicament combinations according to the invention for preparing a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumours, such as for example lymphangiosis carcinomatosa, bronchoalveolar carcinoma and lymphomas.

It is also preferable to use the medicament combinations according to the invention for preparing a pharmaceutical composition for the treatment of interstitial pulmonary diseases selected from among pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic sclerodermy or sarcoidosis, granulomatoses, such as for example Boeck's disease, idiopathic interstitial pneumonia or idiopathic pulmonary fibrosis (IPF).

It is also preferable to use the medicament combinations according to the invention for preparing a pharmaceutical composition for the treatment of cystic fibrosis or mucoviscidosis.

It is also preferable to use the medicament combinations according to the invention for preparing a pharmaceutical composition for the treatment of bronchitis, such as for example bronchitis caused by bacterial or viral infection, allergic bronchitis and toxic bronchitis.

It is also preferable to use the medicament combinations according to the invention for preparing a pharmaceutical composition for the treatment of bronchiectasis.

It is also preferable to use the medicament combinations according to the invention for preparing a pharmaceutical composition for the treatment of ARDS (adult respiratory distress syndrome).

It is also preferable to use the medicament combinations according to the invention for preparing a pharmaceutical composition for the treatment of pulmonary oedema, for example toxic pulmonary oedema after aspiration or inhalation of toxic substances and foreign substances.

It is particularly preferable to use the compounds detailed above for preparing a pharmaceutical composition for the treatment of asthma or COPD. Also of particular importance is the above-mentioned use of medicament combinations according to the invention for preparing a pharmaceutical composition for once-a-day treatment of inflammatory and obstructive respiratory complaints, particularly for the once-a-day treatment of asthma or COPD.

The present invention also relates to the use of therapeutically effective amounts of an active substance 1 in combination with therapeutically effective amounts of active substances 2 and 3 for preparing a pharmaceutical composition for the treatment of one of the above-mentioned diseases.

The present invention also relates to a process for treating one of the above-mentioned diseases, which is characterised in that therapeutically effective amounts of active substance 1 are administered in combination with therapeutically effective amounts of active substances 2 and 3.

Within the scope of the medicament combinations according to the invention, for example, 0.1-1000 μg of a compound 1 may be administered per single dose. Preferably, 0.5-900 μg, particularly preferably 1-800 μg of the compound 1 are administered per single dose.

Without restricting the invention thereto, in the case of 1.8 a dosage range of from 1-50 μg, preferably from 2-25 μg is preferred according to the invention. Particularly preferably, the pharmaceutical compositions according to the invention containing 1.8 are administered in such an amount that 2-10 μg, in case of the fumarate dihydrate particularly preferably 4-10 μg, in case of the hemifumarate monohydrate preferably 2.5-5 μg of the compound 1.8 are administered per single dose.

Without restricting the invention thereto, in the case of 1.23 a dosage range of from 5-100 μg, preferably from 10-75 μg is preferred according to the invention. Particularly preferably, the pharmaceutical compositions according to the invention containing 1.23 are administered in such an amount that 30-60 μg of the compound 1.8, preferably in form of the xinafoate thereof are administered per single dose.

Without restricting the invention thereto, in the case of 1.30 a dosage range of from 1-50 μg, preferably from 2-25 μg is preferred according to the invention. Particularly preferably, the pharmaceutical compositions according to the invention containing 1.8 are administered in such an amount that 2-10 μg are administered per single dose.

Without restricting the invention thereto, in the case of 1.34 a dosage range of from 50-800 μg, preferably from 75-700 μg is preferred according to the invention. Particularly preferably, the pharmaceutical compositions according to the invention containing 1.34 are administered in such an amount that 100-600 μg are administered per single dose

Particularly preferably, the compounds of formula 1 are administered in the above-mentioned dosage ranges in the form of the enantiomerically pure compounds, particularly preferably in the form of the R-enantiomers thereof.

If the compounds of formula 1 are administered in conjunction with an anticholinergic 2, the amount of anticholinergic used will fluctuate considerably depending on the choice of active substance.

Without restricting the invention thereto, in the case of tiotropium 2.1′ amounts of anticholinergic 2 may be administered such that each single dose contains 0.1-80 μg, preferably 0.5-60 μg, particularly preferably about 1-50 μg of 2.1′. For example and without restricting the present invention thereto, 2.5 μg, 5 μg, 10 μg, 18 μg, 20 μg, 36 μg or 40 μg 2.1′ may be administered per single dose. The corresponding amount of salt 2.1 or of any hydrate or solvate used in each case can easily be calculated by the skilled man, depending on the choice of anion. If for example tiotropium bromide is used as the preferred tiotropium salt 2.1 according to the invention, the amounts of the active substance 2.1′ administered per single dose as specified by way of example hereinbefore correspond to the following amounts of 2.1 administered per single dose: 3 μg, 6 μg, 12 μg, 21.7 μg, 24.1 μg, 43.3 μg and 48.1 μg 2.1. In the case of tiotropium 2.1′ the dosages specified above are preferably administered once or twice a day, while administration once a day is particularly preferred according to the invention.

Without restricting the invention thereto, in the case of the cation 2.2′ amounts of anticholinergic 2 may be administered such that each single dose contains 1-500 μg, preferably 5-300 μg, particularly preferably 15-200 μg 2.2′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2.2′ may be administered per single dose. The corresponding amount of salt 2.2 used in each case or of any hydrate or solvate used can easily be calculated by the skilled man, depending on the choice of anion. In the case of oxitropium 2.2′ the dosages specified above are preferably administered one to four times a day, while administration two to three times a day is particularly preferred according to the invention.

Without restricting the invention thereto, in the case of the cation 2.3′ amounts of anticholinergic 2 may be administered such that each single dose contains 1-500 μg, preferably 5-300 μg, particularly preferably 15-200 μg 2.3′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 10 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2.3′ may be administered per single dose. The corresponding amount of salt 2.3 used in each case or of any hydrate or solvate used can easily be calculated by the skilled man, depending on the choice of anion. In the case of flutropium 2.3′ the dosages specified above are preferably administered one to four times a day, while administration two to three times a day is particularly preferred according to the invention.

Without restricting the invention thereto, in the case of the cation 2.4′ amounts of anticholinergic 2 may be administered such that each single dose contains 1-500 μg, preferably 5-300 μg, particularly preferably 20-200 μg 2.4′. For example and without restricting the present invention thereto, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2.4′ may be administered per single dose. The corresponding amount of salt 2.4 used in each case or of any hydrate or solvate used can easily be calculated by the skilled man, depending on the choice of anion. In the case of ipratropium 2.4′ the dosages specified above are preferably administered one to four times a day, while administration two to three times a day, more preferably three times a day, is particularly preferred according to the invention.

Without restricting the invention thereto, in the case of the cation 2.5′ amounts of anticholinergic 2 may be administered such that each single dose contains 1-500 μg, preferably 5-300 μg, particularly preferably 15-200 μg. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2.5′ may be administered per single dose. The corresponding amount of salt 2.5 used in each case or of any hydrate or solvate used can easily be calculated by the skilled man, depending on the choice of anion. In the case of glycopyrronium 2.5′ the dosages specified above are preferably administered one to four times a day, while administration two to three times a day is particularly preferred according to the invention.

Without restricting the invention thereto, in the case of the cation 2.6′ amounts of anticholinergic 2 may be administered such that each single dose contains 1000-6500 μg, preferably 2000-6000 μg, particularly preferably 3000-5500 μg, particularly preferably 4000-5000 μg 2.6′. For example and without restricting the present invention thereto, 3500 μg, 3750 μg, 4000 μg, 4250 μg, 4500 μg, 4750 μg, or 5000 μg of 2.6′ may be administered per single dose. The corresponding amount of salt 2.6 used in each case or of any hydrate or solvate used can easily be calculated by the skilled man, depending on the choice of anion. In the case of trospium 2.6′ the dosages specified above are preferably administered one to four times a day, while administration two to three times a day is particularly preferred according to the invention.

Without restricting the invention thereto, in the case of the cation 2.7′ amounts of anticholinergic 2 may be administered such that each single dose contains 50-1000 μg, preferably 100-800 μg, particularly preferably 200-700 μg, particularly preferably 300-600 μg 2.7′. For example and without restricting the present invention thereto, 300 μg, 350 μg, 400 μg, 450 μg, 500 μg, 550 μg, or 600 μg of 2.7′ may be administered per single dose. The corresponding amount of salt 2.7 used in each case or of any hydrate or solvate used can easily be calculated by the skilled man, depending on the choice of anion. In the case of the cation 2.7′ the dosages specified above are preferably administered one to three times a day, while administration once or twice a day, more preferably once a day, is particularly preferred according to the invention.

Without restricting the invention thereto, in the case of the cations 2.9′ and 2.10′, amounts of anticholinergic 2 may be administered such that each single dose contains 1-500 μg, preferably 5-300 μg, particularly preferably 15-200 μg 2.9′ or 2.10′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2.9′ or 2.10′ may be administered per single dose. The corresponding amount of salt 2.9′ or 2.10′ or of any hydrate or solvate used in each case can easily be calculated by the skilled man, depending on the choice of anion. In the case of the cations 2.9′ or 2.10′ the dosages specified above are preferably administered one to three times a day, while administration once or twice a day, more preferably once a day, is particularly preferred according to the invention.

Without restricting the invention thereto, in the case of the cations 2.11′ to 2.13′ amounts of anticholinergic 2 may be administered such that each single dose contains 1-500 μg, preferably 5-300 μg, particularly preferably 10-200 μg 2.11′, 2.12′ or 2.13′. For example and without restricting the present invention thereto, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2.11′, 2.12′ or 2.13′ may be administered per single dose. The corresponding amount of salt 2.11, 2.12 or 2.13 or of any hydrate or solvate used in each case can easily be calculated by the skilled man, depending on the choice of anion.

In the case of the cations 2.11, 2.12 or 2.13 the dosages specified above are preferably administered one to three times a day, while administration once or twice a day, more preferably once a day, is particularly preferred according to the invention.

In the combinations according to the invention the PDE IV-inhibitor 3 is preferably administered in such an amount that about 1-10000 μg 3 are administered per single dose. Preferably, amounts of 3 are administered such that each single dose contains 10-5000 μg, preferably 50-2500 μg, particularly preferably 100-1000 μg of 3. For example and without restricting the present invention thereto, 100 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg or 1000 μg of 3 may be administered per single dose. In the event that acid addition salts of 3 are used, the corresponding amount of salt used can easily be calculated by the skilled man from the values given hereinbefore, depending on the choice of acid.

The active substance components 1, 2 and 3 may be administered—together or separately—in each case by inhalation or by oral, parenteral or some other route, in known manner, in substantially conventional formulations such as for example plain or coated tablets, pills, granules, aerosols, syrups, emulsions, suspensions, powders and solutions, using inert, non-toxic, pharmaceutically suitable carriers or solvents.

Suitable preparations for administering the compounds 1, 2 and 3 include tablets, capsules, suppositories, solutions, powders, etc. The proportion of pharmaceutically active compound or compounds should be in the range from 0.05 to 90% by weight, preferably 0.1 to 50% by weight of the total composition. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations of active substances according to the invention may additionally contain a sweetener such as saccharine, cyclamate, glycerol or sugar and a flavour enhancer, e.g. a flavouring such as vanilline or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral administration the tablets may, of course, contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

Preferably, even when the components 1, 2 and 3 are administered separately, at least components 1 and 2 are administered by inhalation. Component 3 may also be administered for example by oral or parenteral route using formulations conventional in the art such as plain or coated tablets, pills, granules, aerosols, syrups, emulsions, suspensions, powders and solutions, using inert, non-toxic, pharmaceutically suitable carriers or solvents.

Preferably, however, the medicament combinations according to the invention are administered by inhalation by means of a single preparation containing the active substances 1, 2 and 3 or by means of separate preparations each containing only one of the active substances 1, 2 and 3 suitable for administration by inhalation.

Inhalable preparations include inhalable powders, propellant-containing metered dose aerosols or propellant-free inhalable solutions. Inhalable powders according to the invention containing the combination of active substances 1, 2 and 3 may consist of the active substances on their own or of a mixture of the active substances with physiologically acceptable excipients. Within the scope of the present invention, the term propellant-free inhalable solutions also includes concentrates or sterile inhalable solutions ready for use. The preparations according to the invention may contain the combination of active substances 1, 2 and 3 either together in one formulation or in two separate formulations. These formulations which may be used within the scope of the present invention are described in more detail in the next part of the specification.

A) Inhalable Powder Containing the Combinations of Active Substances According to the Invention:

The inhalable powders according to the invention may contain 1, 2 and 3 either on their own or in admixture with suitable physiologically acceptable excipients. If the active substances 1, 2 and 3 are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose, trehalose), oligo- and polysaccharides (e.g. dextrans), polyalcohols (e.g. sorbitol, mannitol, xylitol), salts (e.g. sodium chloride, calcium carbonate) or mixtures of these excipients with one another. Preferably, mono- or disaccharides are used, while the use of lactose, trehalose or glucose is preferred, particularly, but not exclusively, in the form of their hydrates.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipients mentioned above. These finer excipients are also selected from the group of possible excipients listed hereinbefore. Finally, in order to prepare the inhalable powders according to the invention, micronised active substance 1, 2 and 3, preferably with an average particle size of 0.5 to 10 μm, more preferably from 1 to 6 μm, is added to the excipient mixture. Processes for producing the inhalable powders according to the invention by grinding and micronising and finally mixing the ingredients together are known from the prior art. The inhalable powders according to the invention may be prepared and administered either in the form of a single powder mixture which contains 1, 2 and 3 or in the form of separate inhalable powders which contain only 1, 2 and/or 3.

The inhalable powders according to the invention may be administered using inhalers known from the prior art. Inhalable powders according to the invention which contain a physiologically acceptable excipient in addition to 1, 2 and 3 may be administered, for example, by means of inhalers which deliver a single dose from a supply using a measuring chamber as described in U.S. Pat. No. 4,570,630A, or by other means as described in DE 36 25 685 A. The inhalable powders according to the invention which contain 1, 2 and 3 optionally in conjunction with a physiologically acceptable excipient may be administered, for example, using the inhaler known by the name TURBUHALER® or using inhalers as disclosed for example in EP 237507 A. Preferably, the inhalable powders according to the invention which contain physiologically acceptable excipient in addition to 1, 2 and 3 are packed into capsules (to produce so-called inhalettes) which are used in inhalers as described, for example, in WO 94/28958.

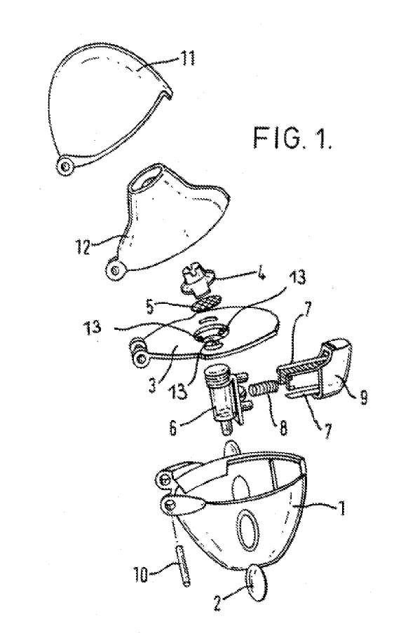

A particularly preferred inhaler for using the pharmaceutical combination according to the invention in capsules is shown in FIG. 1.

This inhaler (HANDIHALER®) for inhaling powdered pharmaceutical compositions from capsules is characterised by a housing 1 containing two windows 2, a deck 3 in which there are air inlet ports and which is provided with a screen 5 secured by a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut, and air through-holes 13 for adjusting the flow resistance.

If the inhalable powders according to the invention are to be packaged in capsules, in accordance with the preferred method of administration described above, the capsules should preferably contain from 1 to 30 mg each. According to the invention they contain either together or separately the dosages per single dose specified for 1 and 2 hereinbefore.

B) Propellant Gas-Driven Inhalation Aerosols Containing the Combinations of Active Substances According to the Invention:

Inhalation aerosols containing propellant gas according to the invention may contain substances 1, 2 and 3 dissolved in the propellant gas or in dispersed form. 1, 2 and 3 may be present in separate formulations or in a single preparation, in which 1, 2 and 3 are either both dissolved, both dispersed or only one component is dissolved and the other is dispersed. The propellant gases which may be used to prepare the inhalation aerosols according to the invention are known from the prior art. Suitable propellant gases are selected from among hydrocarbons such as n-propane, n-butane or isobutane and halohydrocarbons such as preferably chlorinated and fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG11, TG12, TG134a (1,1,1,2-tetrafluoroethane), TG227 (1,1,1,2,3,3,3-heptafluoropropane) and mixtures thereof, the propellant gases TG134a, TG227 and mixtures thereof being preferred.

The propellant-driven inhalation aerosols according to the invention may also contain other ingredients such as co-solvents, stabilisers, surfactants, antioxidants, lubricants and pH adjusters. All these ingredients are known in the art.

The inhalation aerosols containing propellant gas according to the invention may contain up to 5 wt.-% of active substance 1, 2 and/or 3. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of active substance 1, 2 and/or 3.

If the active substances 1, 2 and/or 3 are present in dispersed form, the particles of active substance preferably have an average particle size of up to 10 μm, preferably from 0.1 to 6 μm, more preferably from 1 to 5 μm.

The propellant-driven inhalation aerosols according to the invention mentioned above may be administered using inhalers known in the art (MDIs=metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterised in that they contain the propellant gas-containing aerosols described above according to the invention.

The present invention also relates to cartridges which are fitted with a suitable valve and can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

C) Propellant-Free Inhalable Solutions or Suspensions Containing the Combinations of Active Substances 1 and 2 According to the Invention:

Propellant-free inhalable solutions according to the invention contain for example aqueous or alcoholic, preferably ethanolic solvents, possibly ethanolic solvents in admixture with aqueous solvents. In the case of aqueous/ethanolic solvent mixtures the relative proportion of ethanol to water is not restricted, but the maximum limit is up to 70 percent by volume, more particularly up to 60 percent by volume of ethanol. The remainder of the volume is made up of water. The solutions or suspensions containing 1, 2 and 3, separately or together, are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid, etc. Preferred inorganic acids are hydrochloric acid and sulphuric acid. It is also possible to use the acids which have already formed an acid addition salt with one of the active substances. Of the organic acids, ascorbic acid, fumaric acid and citric acid are preferred. If desired, mixtures of the above acids may also be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

According to the invention, the addition of edetic acid (EDTA) or one of the known salts thereof, sodium edetate, as stabiliser or complexing agent is unnecessary in the present formulation. Other embodiments may contain this compound or these compounds. In a preferred embodiment the content based on sodium edetate is less than 100 mg/100 ml, preferably less than 50 mg/100 ml, more preferably less than 20 mg/100 ml. Generally, inhalable solutions in which the content of sodium edetate is from 0 to 10 mg/100 ml are preferred.

Co-solvents and/or other excipients may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the combination of active substances 1, 2 and 3, only benzalkonium chloride and sodium edetate. In another preferred embodiment, no sodium edetate is present.

The propellant-free inhalable solutions according to the invention are administered in particular using inhalers of the kind which are capable of nebulising a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 10 and 30 μL of active substance solution can be nebulised in preferably one spray action to form an aerosol with an average particle size of less than 20 μm, preferably less than 10 μm, such that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf in particular FIGS. 6a and 6b). The nebulisers (devices) described therein are known by the name Respimat®.

We claim:

1. A pharmaceutical composition comprising at least one betamimetic 1, at least one anticholinergic 2 and at least one PDEIV-inhibitor 3, and optionally a pharmaceutically acceptable excipient.

2. A pharmaceutical composition according to claim 1, wherein the betamimetic 1 is selected from albuterol (1.1), bambuterol (1.2), bitolterol (1.3), broxaterol (1.4), carbuterol (1.5), clenbuterol (1.6), fenoterol (1.7), formoterol (1.8), hexoprenaline (1.9), ibuterol (1.10), isoetharine (1.11), isoprenaline (1.12), levosalbutamol (1.13), mabuterol (1.14), meluadrine (1.15), metaproterenol (1.6), orciprenaline (1.17), pirbuterol (1.18), procaterol (1.19), reproterol (1.20), TD 3327 (1.21), ritodrine (1.22), salmeterol (1.23), salmefamol (1.24), soterenot (1.25), sulphonterol (1.26), tiaramide (1.27), terbutaline (1.28), tolubuterol (1.29), CHF-4226 (=TA 2005 or carmoterol; 1.30), HOKU-81 (1.31), KUL-1248 (1.32), 3-(4-{6-[2-Hydroxy-2-(4-hydroxy-3-hydroxymethyl-phenyl)-ethylamino]-hexyloxy}-butyl)-benzenesulfoneamide (1.33), 5-[2-(5,6-Diethyl-indan-2-ylamino)-1-hydroxy-ethyl]-8-hydroxy-1H-quinolin-2-one (1.34), 4-hydroxy-7-[2-{[2-{[3-(2-phenylethoxy)propyl]sulphonyl}ethyl]-amino}ethyl]-2(3H)-benzothiazolone (1.35), 1-(2-fluoro-4-hydroxyphenyl)-2-[4-(1-benzimidazolyl)-2-methyl-2-butylamino]ethanol (1.36), 1-[3-(4-methoxybenzyl-amino)-4-hydroxyphenyl]-2-[4-(1-benzimidazolyl)-2-methyl-2-butylamino]ethanol (1.37), 1-[2H-5-hydroxy-3-oxo-4H-1,4-benzoxazin-8-yl]-2-[3-(4-N,N-dimethylaminophenyl)-2-methyl-2-propylamino]ethanol (1.38), 1-[2H-5-hydroxy-3-oxo-4H-1,4-benzoxazin-8-yl]-2-[3-(4-methoxyphenyl)-2-methyl-2-propylamino]ethanol (1.39), 1-[2H-5-hydroxy-3-oxo-4H-1,4-benzoxazin-8-yl]-2-[3-(4-n-butyloxyphenyl)-2-methyl-2-propylamino]ethanol (1.40), 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol (1.41), 5-hydroxy-8-(1-hydroxy-2-isopropylaminobutyl)-2H-1,4-benzoxazin-3-(4H)-one (1.42), 1-(4-amino-3-chloro-5-trifluormethylphenyl)-2-tert.-butylamino)ethanol (1.43), 1-(4-ethoxycarbonylamino-3-cyano-5-fluorophenyl)-2-(tert.-butylamino)ethanol (1.44), and N-[2-Hydroxy-5-(1-hydroxy-2-{2-[4-(2-hydroxy-2-phenyl-ethylamino)-phenyl]-ethylamino}-ethyl)-phenyl]-formamide (1.45), or a pharmacologically acceptable acid addition salt or hydrate thereof.

3) A pharmaceutical composition according to claim 1, wherein the anticholinergic (2) is selected from tiotropium salts (2.1), oxitropium salts (2.2), flutropium salts (2.3), ipratropium salts (2.4), glycopyrronium salts (2.5), trospium salts (2.6), an anticholinergic of formula 2.7 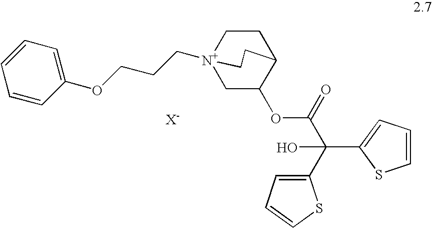
wherein
X⁻ denotes an anion with a single negative charge, preferably an anion selected from among fluoride, chloride, bromide, iodide, sulphate, phosphate, methanesulphonate, nitrate, maleate, acetat, citrate, fumarate, tartrate, oxalate, succinate, benzoate and p-toluenesulphonate,
or a hydrate thereof,
and an anticholinergic of formula 2.8 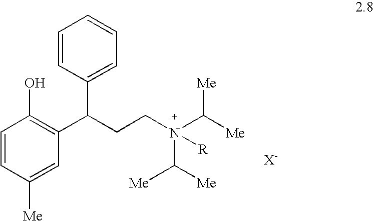
wherein r denotes either methyl (2.8.1) or ethyl (8.2) and wherein X⁻ may have the meanings mentioned hereinbefore, or a hydrate thereof.

4. A pharmaceutical composition according to claim 1, wherein the anticholinergic (2) is selected from anticholinergics of formula 2.9 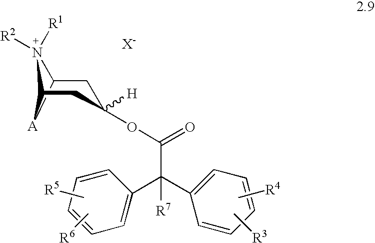
wherein
A denotes a double-bonded group selected from the groups 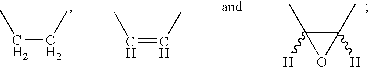
X⁻ denotes an anion with a single negative charge;
R¹ and R² which may be identical or different denote a group selected from methyl, ethyl, n-propyl and iso-propyl, which may optionally be substituted by hydroxy or fluorine;
R³, R⁴, R⁵ and R⁶, which may be identical or different, denote hydrogen, methyl, ethyl, methyloxy, ethyloxy, hydroxy, fluorine, chlorine, bromine, CN, CF₃ or NO₂;
R⁷ denotes hydrogen, methyl, ethyl, methyloxy, ethyloxy, —CH₂—F, —CH₂—CH₂—F, —O—CH₂—F, —O—CH₂—CH₂—F, —CH₂—OH, —CH₂—CH₂—OH, CF₃, —CH₂—OMe, —CH₂—CH₂-OMe, —CH₂—OEt, —CH₂—CH₂-OEt, —O—COMe, —O—COEt, —O—COCF₃, —O—COCF₃, fluorine, chlorine or bromine,
or a hydrate thereof.

5. A pharmaceutical composition according to claim 1, wherein the anticholinergic (2) is selected from the compounds of formula 2.10 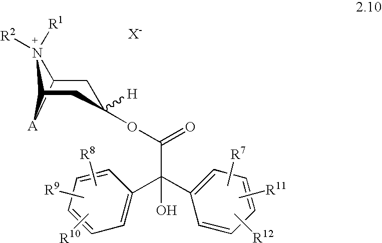
wherein
A denotes a double-bonded group selected from the groups 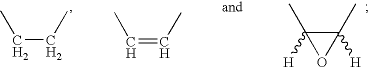
X⁻ denotes an anion with a single negative charge;
R¹ and R² which may be identical or different denote a group selected from methyl, ethyl, n-propyl and iso-propyl, which may optionally be substituted by hydroxy or fluorine;
R⁷, R⁸, R⁹, R¹⁰, R¹¹ and R¹², which may be identical or different, denote hydrogen, methyl, ethyl, methyloxy, ethyloxy, hydroxy, fluorine, chlorine, bromine, CN, CF₃ or NO₂, while at least one of the groups R⁷, R⁸, R⁹, R¹⁰, R¹¹ and R¹² is other than hydrogen,
or a hydrate thereof.

6. A pharmaceutical composition according to claim 1, wherein the anticholinergic (2) is selected from anticholinergics of formula 2.11 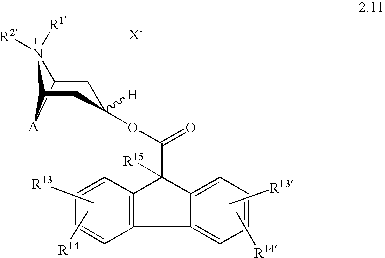
wherein
A denotes a double-bonded group selected from the groups 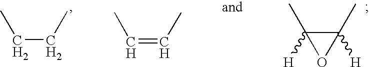
X⁻ denotes an anion with a single negative charge;
R¹⁵ denotes hydrogen, hydroxy, methyl, ethyl, —CF₃, CHF₂ or fluorine;
R^{1′} and R^{2′} which may be identical or different, denote C₁-C₅-alkyl, which may optionally be substituted by C₃-C₆-cycloalkyl, hydroxy or halogen, or
R^{1′} and R^{2′} together denote a —C₃-C₅-alkylene bridge;
R¹³, R¹⁴, R^{13′} and R^{14′} which may be identical or different, denote hydrogen, —C₁-C₄-alkyl, —C₁-C₄-alkyloxy, hydroxy, —CF₃, —CHF₂, CN, NO₂ or halogen,
or a hydrate thereof.

7. A pharmaceutical composition according to claim 1, wherein the anticholinergic (2) is selected from anticholinergics of formula 2.12 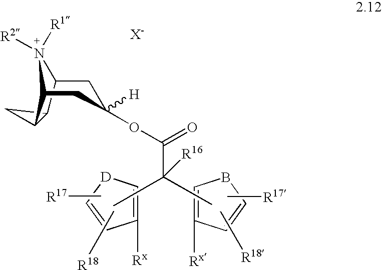
wherein X⁻ denotes an anion with a single negative charge;
D and B which may be identical or different, denote O, S, NH, CH₂, CH═CH or N(C₁-C₄-alkyl);
R¹⁶ denotes hydrogen, hydroxy, —C₁-C₄-alkyl, —C₁-C₄-alkyloxy, —C₁-C₄-alkylene-halogen, —O—C₁-C₄-alkylene-halogen, —C₁-C₄-alkylene-OH, —CF₃, CHF₂, —C₁-C₄-alkylene-C₁-C₄-alkyloxy, —O—COC₁-C₄-alkyl, —O—COC₁-C₄-alkylene-halogen, —C₁-C₄-alkylene-C₃-C₆-cycloalkyl, —O—COCF₃ or halogen;
R^{1″} and R^{2″} which may be identical or different, denote —C₁-C₅-alkyl, which may optionally be substituted by —C₃-C₆-cycloalkyl, hydroxy or halogen,
or
R^{1″} and R^{2″} together denote a —C₃-C₅-alkylene bridge;
R¹⁷, R¹⁸, R^{17′} and R^{18′}, which may be identical or different, denote hydrogen, —C₁-C₄-alkyl, —C₁-C₄-alkyloxy, hydroxy, —CF₃, —CHF₂, CN, NO₂ or halogen;
R^{X} and R^{X′} which may be identical or different, denote hydrogen, —C₁-C₄-alkyl, —C₁-C₄-alkyloxy, hydroxy, —CF₃, —CHF₂, CN, NO₂ or halogen,
or
R^{X} and R^{X′} together denote a single bond or one of the double-bonded groups O, S, NH, CH₂, CH₂—CH₂, N(C₁-C₄-alkyl), CH(C₁-C₄-alkyl) and —C(C₁-C₄-alkyl)₂,
or a hydrate thereof.

8) A pharmaceutical composition according to claim 1, wherein the anticholinergic (2) is selected from anticholinergics of formula 2.13 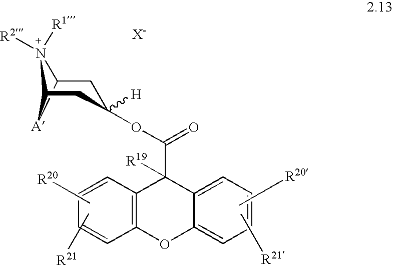
wherein X⁻ denotes an anion with a single negative charge;
A′ denotes a double-bonded group selected from 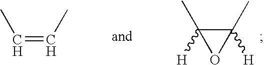
R¹⁹ denotes hydroxy, methyl, hydroxymethyl, ethyl, —CF₃, CHF₂ or fluorine;
R^{1′″} and R^{2′″} which may be identical or different, denote C₁-C₅-alkyl, which may optionally be substituted by C₃-C₆-cycloalkyl, hydroxy or halogen,
or
R^{1′″} and R^{2′″} together denote a —C₃-C₅-alkylene bridge;
R²⁰, R²¹, R^{20′} and R^{21′} which may be identical or different, denote hydrogen, —C₁-C₄-alkyl, —C₁-C₄-alkyloxy, hydroxy, —CF₃, —CHF₂, CN, NO₂ or halogen,
or a hydrate thereof.

9. A pharmaceutical composition according to claim 1, wherein the PDE IV-inhibitor 3 is a compound selected from enprofyllin (3.1), theophyllin (3.2), roflumilast (3.3), ariflo (Cilomilast, 3.4)), CP-325,366 (3.5), BY343 (3.6), D-4396 (Sch-351591, 3.7)), AWD-12-281 (GW-842470, 3.8)), N-(3,5-dichloro-1-oxo-pyridin-4-yl)-4-difluoromethoxy-3-cyclopropylmethoxybenzamide (3.9), NCS-613 (3.10), pumafentine (3.11), (−)p-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamide (3.12), (R)-(+)-1-(4-bromobenzyl)-4-[(3-cyclopentyloxy)-4-methoxyphenyl]-2-pyrrolidone (3.13), 3-(cyclopentyloxy-4-methoxyphenyl)-1-(4-N′-[N-2-cyano-S-methyl-isothioureido]benzyl)-2-pyrrolidone (3.14), cis[4-cyano-4-(3-cyclopentyloxy-4-methoxyphenyl)cyclohexane-1-carboxylic acid] (3.15), 2-carbomethoxy-4-cyano-4-(3-cyclopropylmethoxy-4-difluoromethoxyphenyl)cyclohexan-1-one (3.16), cis[4-cyano-4-(3-cyclopropylmethoxy-4-difluoromethoxyphenyl)cyclohexan-1-ol] (3.17), (R)-(+)-ethyl[4-(3-cyclopentyloxy-4-methoxyphenyl)pyrrolidin-2-ylidene]acetate (3.18), (S)-(−)-ethyl[4-(3-cyclopentyloxy-4-methoxyphenyl)pyrrolidin-2-ylidene]acetate (3.19), 4-(3-cyclopentyloxy-4-methoxyphenyl)-3-(1-hydroxy-ethyl)-3-methyl-pyrrolidine-1-carboxylic acid methyl ester (=IC 485, 3.20), CDP840 (3.21), Bay-198004 (3.22), D-4418 (3.23), PD-168787 (3.24), T-440 (3.25), T-2585 (3.26), arofyllin (3.27), atizoram (3.28), V-11294A (3.29), C1-1018 (3.30), CDC-801 (3.31), CDC-3052 (3.32), D-22888 (3.33), YM-58997 (3.34), Z-15370 (3.35), 9-cyclopentyl-5,6-dihydro-7-ethyl-3-(2-thienyl)-9H-pyrazolo[3,4-c]-1,2,4-triazolo[4,3-a]pyridine (3.36), 9-cyclopentyl-5,6-dihydro-7-ethyl-3-(tert-butyl)-9H-pyrazolo[3,4-c]-1,2,4-triazolo[4,3-a]pyridine (3.37), and tetomilast (3.38), or a pharmacologically acceptable acid addition salt, solvate or hydrate thereof.

10. A pharmaceutical composition according to claim 1, comprising:
a) at least one betamimetic selected from formoterol (1.8), salmeterol (1.23), CHF-4226 (=TA 2005 or carmoterol; 1.30), 3-(4-{6-[2-Hydroxy-2-(4-hydroxy-3-hydroxymethyl-phenyl)-ethylamino]-hexyloxy}-butyl)-benzenesulfoneamide (1.33), 5-[2-(5,6-Diethyl-indan-2-ylamino)-1-hydroxy-ethyl]-8-hydroxy-1H-quinolin-2-one (1.34), and N-[2-Hydroxy-5-(1-hydroxy-2-{2-[4-(2-hydroxy-2-phenyl-ethylamino)-phenyl]-ethylamino}-ethyl)-phenyl]-formamide (1.45),
b) at least one anticholinergic selected from tiotropium salts (2.1), ipratropium salts (1.4, glycopyrronium salts (2.5), an anticholinergic of formula 2.7 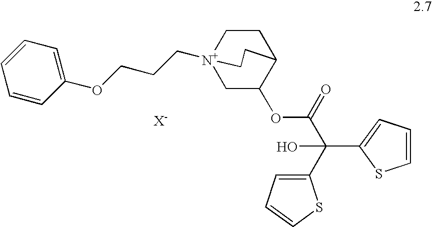
wherein
X⁻ denotes an anion with a single negative charge, preferably an anion selected from among fluoride, chloride, bromide, iodide, sulphate, phosphate, methanesulphonate, nitrate, maleate, acetat, citrate, fumarate, tartrate, oxalate, succinate, benzoate and p-toluenesulphonate,
or a hydrate thereof,
tropenol 2,2-diphenylpropionate methobromide (29.1),
scopine 2,2-diphenylpropionate methobromide (2.9.2),
cyclopropyltropine benzilate methobromide (2.12.1), and
cyclopropyltropine 2,2-diphenylpropionate methobromide (2.12.2);
and c) at least one PDEIV inihibitor selected from roflumilast (3.3), AWD-12-281 (GW-842470, 3.8), and Z-15370 (3.35);
and optionally a pharmaceutically acceptable excipient.

11. A pharmaceutical composition according to claim 10, comprising:
a) at least one betamimetic selected from CHF-4226 (=TA 2005 or carmoterol; 1.30), 3-(4-{6-[2-Hydroxy-2-(4-hydroxy-3-hydroxymethyl-phenyl)-ethylamino]-hexyloxy}-butyl)-benzenesulfoneamide (1.33), and 5-[2-(5,6-Diethyl-indan-2-ylamino)-1-hydroxy-ethyl]-8-hydroxy-1H-quinolin-2-one (1.34);
b) at least one anticholinergic selected from tiotropium salts (2.1), glycopyrronium salts (2.5), an anticholinergic of formula 2.7 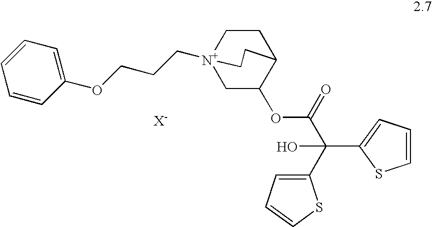
wherein
X⁻ denotes an anion with a single negative charge, preferably an anion selected from among fluoride, chloride, bromide, iodide, sulphate, phosphate, methanesulphonate, nitrate, maleate, acetat, citrate, fumarate, tartrate, oxalate, succinate, benzoate and p-toluenesulphonate,
or a hydrate thereof,
tropenol 2,2-diphenylpropionate methobromide (29.1), and
scopine 2,2-diphenylpropionate methobromide (2.9.2);
and c) at least one PDEIV inihibitor selected from roflumilast (3.3), AWD-12-281 (GW-842470, 3.8), and Z-15370 (3.35);
and optionally a pharmaceutically acceptable excipient.

12. A pharmaceutical composition according to claim 1, further comprising a pharmaceutically acceptable carrier or solvent.

13. A pharmaceutical composition according to claim 1, in a form of a formulation suitable for inhalation.

14. A pharmaceutical composition according to claim 13, wherein the formulation is selected from inhalable powders, propellant-driven metered-dose aerosols and propellant-free inhalable solutions or suspensions.

15. A method for treating inflammatory and obstructive respiratory complaints, or for inhibiting premature labour in midwifery (tocolysis), or for restoring sinus rhythm in the heart in atrioventricular block, or for correcting bradycardic heart rhythm disorders (antiarrhythmic), or for treating circulatory shock (vasodilatation and increasing the heart volume), or for treating skin irritations and inflammation, in a subject comprising administering to said subject a therapeutically effective amount of a pharmaceutical composition according to claim 1.

16. A method according to claim 15, directed to the treatment of asthma or COPD.
